SEQUENCE LISTING (253 sequences)

SEQ ID NO: 1 - protein, 188 aa, unidentified [sequence omitted]

SEQ ID NO: 2 - protein, 189 aa, unidentified [sequence omitted]

SEQ ID NO: 3 - protein, 99 aa, Homo sapiens [sequence omitted]

SEQ ID NO: 4 - protein, 99 aa, synthetic construct
  REGION 1..99: note = synthetic sequence
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 5 - protein, 227 aa, synthetic construct
  REGION 1..227: note = synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, synthetic construct
  REGION 1..227: note = synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 14 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 15 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 16 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 17 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 275 aa, Homo sapiens [sequence omitted]

SEQ ID NO: 19 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 20 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 21 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 22 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 23 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 24 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 25 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 26 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 27 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 28 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 29 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 30 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 31 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 32 - protein, 275 aa, synthetic construct
  REGION 1..275: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 33 - protein, 275 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 34 - protein, 275 aa, synthetic construct
  REGION 1..275: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 35 - protein, 275 aa, synthetic construct
  REGION 1..275: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 36 - protein, 275 aa, synthetic construct
  REGION 1..275: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 37 - protein, 275 aa, synthetic construct
  REGION 1..275: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 38 - protein, 276 aa, synthetic construct
  REGION 1..276: note = synthetic sequence
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 39 - protein, 276 aa, synthetic construct [sequence omitted]

SEQ ID NO: 40 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 41 - protein, 276 aa, synthetic construct
  REGION 1..276: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 42 - protein, 276 aa, synthetic construct
  REGION 1..276: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 43 - protein, 276 aa, synthetic construct
  REGION 1..276: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 44 - protein, 276 aa, synthetic construct
  REGION 1..276: note = synthetic sequence
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 45 - protein, 276 aa, synthetic construct
  REGION 1..276: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 46 - protein, 276 aa, synthetic construct
  REGION 1..276: note = synthetic sequence
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 47 - protein, 276 aa, synthetic construct
  REGION 1..276: note = synthetic sequence
  VARIANT 9: note = Xaa is phenylalanine, tyrosine, serine, or threonine
  VARIANT 44: note = Xaa is lysine or arginine
  VARIANT 62: note = Xaa is glutamine, glycine, glutamic acid, or arginine
  VARIANT 63: note = Xaa is asparagine or glutamic acid
  VARIANT 65: note = Xaa is arginine or glycine
  VARIANT 66: note = Xaa is asparagine or lysine
  VARIANT 67: note = Xaa is methionine or valine
  VARIANT 70: note = Xaa is histidine or glutamine
  VARIANT 73: note = Xaa is threonine or isoleucine
  VARIANT 74: note = Xaa is aspartic acid or histidine
  VARIANT 76: note = Xaa is alanine, valine, or glutamic acid
  VARIANT 77: note = Xaa is asparagine or aspartic acid
  VARIANT 79: note = Xaa is glycine or arginine
  VARIANT 80: note = Xaa is threonine or arginine
  VARIANT 81: note = Xaa is leucine or alanine
  VARIANT 82: note = Xaa is arginine or leucine
  VARIANT 83: note = Xaa is glycine ir arginine
  VARIANT 90: note = Xaa is alanine or aspartic acid
  VARIANT 95: note = Xaa is isoleucine, leucine, or valine
  VARIANT 97: note = Xaa is isoleucine, arginine, or methionine
  VARIANT 99: note = Xaa is phenylalanine or tyrosine
  VARIANT 105: note = Xaa is serine or proline
  VARIANT 107: note = Xaa is tryptophan or glycine
  VARIANT 114: note = Xaa is arginine, histidine, or glutamine
  VARIANT 116: note = Xaa is aspartic acid or tyrosine
  VARIANT 127: note = Xaa is asparagine or lysine
  VARIANT 142: note = Xaa is threonine or isoleucine
  VARIANT 144: note = Xaa is lysine or glutamine
  VARIANT 145: note = Xaa is arginine or histidine
  VARIANT 149: note = Xaa is alanine or threonine
  VARIANT 150: note = Xaa is alanine or valine
  VARIANT 151: note = Xaa is histidine or arginine
  VARIANT 156: note = Xaa is arginine, leucine, glutamine, or tryptophan
  VARIANT 158: note = Xaa is valine or alanine
  VARIANT 161: note = Xaa is aspartic acid or glutamic acid
  VARIANT 163: note = Xaa is arginine or threonine
  VARIANT 166: note = Xaa is aspartic acid or glutamic acid
  VARIANT 167: note = Xaa is tryptophan or glycine
  VARIANT 184: note = Xaa is proline or alanine
  VARIANT 193: note = Xaa is proline or alanine
  VARIANT 194: note = Xaa is valine or isoleucine
  VARIANT 207: note = Xaa is serine or glycine
  VARIANT 246: note = Xaa is alanine or serine
  VARIANT 253: note = Xaa is glutamine or glutamic acid
  VARIANT 276: note = Xaa is proline or leucine
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 48 - protein, 276 aa, synthetic construct [sequence omitted]

SEQ ID NO: 49 - protein, 276 aa, synthetic construct
  REGION 1..276: note = synthetic sequence
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 50 - protein, 276 aa, synthetic construct
  REGION 1..276: note = synthetic sequence
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 51 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 52 - protein, 276 aa, synthetic construct
  REGION 1..276: note = synthetic sequence
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 53 - protein, 276 aa, synthetic construct
  REGION 1..276: note = synthetic sequence
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 54 - protein, 276 aa, synthetic construct
  REGION 1..276: note = synthetic sequence
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 55 - protein, 276 aa, synthetic construct
  REGION 1..276: note = synthetic sequence
  VARIANT 9: note = Xaa is histidine, tyrosine, or aspartic acid
  VARIANT 11: note = Xaa is alanine or serine
  VARIANT 12: note = Xaa is methionine or valine
  VARIANT 24: note = Xaa is alanine, serine, or threonine
  VARIANT 32: note = Xaa is glutamine or leucine
  VARIANT 41: note = Xaa is alanine or threonine
  VARIANT 45: note = Xaa is glutamic acid, methionine, lysine, or threonine
  VARIANT 46: note = Xaa is alanine or threonine
  VARIANT 63: note = Xaa is glutamic acid or asparagine
  VARIANT 66: note = Xaa is isoleucine or lysine
  VARIANT 67: note = Xaa is tyrosine, phenylalanie, serine, or cystine
  VARIANT 70: note = Xaa is asparagine or glutamine
  VARIANT 71: note = Xaa is alanine or threonine
  VARIANT 74: note = Xaa is aspartic acid or tyrosine
  VARIANT 76: note = Xaa is glutamic acid or valine
  VARIANT 77: note = Xaa is serine or asparagine
  VARIANT 80: note = Xaa is threonine, asparagine, or isoleucine
  VARIANT 81: note = Xaa is alanine or leucine
  VARIANT 82: note = Xaa is leucine or arginine
  VARIANT 83: note = Xaa is arginine or glycine
  VARIANT 94: note = Xaa is threonine or isoleucine
  VARIANT 95: note = Xaa is leucine or isoleucine
  VARIANT 97: note = Xaa is arginine or serine
  VARIANT 131: note = Xaa is arginine or serine
  VARIANT 143: note = Xaa is Ser or Thr
  VARIANT 147: note = Xaa is Leu or Trp
  VARIANT 152: note = Xaa is Glu or Val
  VARIANT 156: note = Xaa is Arg, Asp, or Leu
  VARIANT 158: note = Xaa Ala or Thr
  VARIANT 163: note = Xaa is leucine, glutamic acid, or threonine
  VARIANT 177: note = Xaa is glutamic acid or aspartic acid
  VARIANT 178: note = Xaa is lysine or threonine
  VARIANT 180: note = Xaa is glutamic acid or glutamine
  VARIANT 194: note = Xaa is isoleucine or valine
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 56 - protein, 276 aa, synthetic construct
  REGION 1..276: note = synthetic sequence
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 57 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 58 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 59 - protein, 276 aa, synthetic construct
  REGION 1..276: note = synthetic sequence
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 60 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 61 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 62 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 63 - protein, 276 aa, synthetic construct
  REGION 1..276: note = synthetic sequence
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 64 - protein, 276 aa, synthetic construct
  REGION 1..276: note = synthetic sequence
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 65 - protein, 276 aa, synthetic construct
  REGION 1..276: note = synthetic sequence
  VARIANT 1: note = Xaa is cysteine or glycine
  VARIANT 6: note = Xaa is arginine or lysine
  VARIANT 9: note = Xaa is phenylalanine, tyrosine, serine, or aspartic acid
  VARIANT 14: note = Xaa is arginine or tryptophan
  VARIANT 21: note = Xaa is histidine or arginine
  VARIANT 24: note = Xaa is alanine or serine
  VARIANT 35: note = Xaa is glutamine or arginine
  VARIANT 49: note = Xaa is alanine or arginine
  VARIANT 66: note = Xaa is asparagine, or lysine
  VARIANT 73: note = Xaa is threonine or alanine
  VARIANT 77: note = Xaa is serine or asparagine
  VARIANT 80: note = Xaa is asparagine or lysine
  VARIANT 90: note = Xaa is alanine or aspartic acid
  VARIANT 91: note = Xaa is glycine or arginine
  VARIANT 94: note = Xaa is threonine or isoleucine
  VARIANT 95: note = Xaa is leucine or isoleucine
  VARIANT 97: note = Xaa is tryptophan or arginine
  VARIANT 99: note = Xaa is cysteine, tyrosine, phenylalanine, or serine
  VARIANT 103: note = Xaa is leucine or valine
  VARIANT 113: note = Xaa is tyrosine or histidine
  VARIANT 114: note = Xaa is aspartic acid or asparagine
  VARIANT 116: note = Xaa is tyrosine, phenylalanine, serine, or leucine
  VARIANT 147: note = Xaa is leucine or tryptophan
  VARIANT 152: note = Xaa is glutamic acid, alanine, or threonine
  VARIANT 156: note = Xaa is arginine, leucine, or tryptophan
  VARIANT 163: note = Xaa is leucine or threonine
  VARIANT 173: note = Xaa is glutamic acid or lysine
  VARIANT 177: note = Xaa is glutamic acid or lysine
  VARIANT 184: note = Xaa is histidine or proline
  VARIANT 194: note = Xaa is arginine or valine
  VARIANT 219: note = Xaa is tryptophan or arginine
  VARIANT 248: note = Xaa is valine or methionine
  VARIANT 253: note = Xaa is glutamic acid or lysine
  VARIANT 261: note = Xaa is methionine or valine
  VARIANT 267: note = Xaa is proline or glutamine
  VARIANT 273: note = Xaa is arginine or serine
  VARIANT 275: note = Xaa is proline or glycine
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 66 - protein, 284 aa, synthetic construct
  REGION 1..284: note = synthetic sequence
  VARIANT 89: note = Xaa is Lys or Glu
  VARIANT 107: note = Xaa is Arg or Gly
  VARIANT 157: note = Xaa is Arg or Gly
  VARIANT 158: note = Xaa is Ala or Val
  VARIANT 255: note = Xaa is Gln or Pro
  VARIANT 267: note = Xaa is Pro or Ser
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 67 - protein, 284 aa, synthetic construct
  REGION 1..284: note = synthetic sequence
  VARIANT 7: note = Xaa is Tyr or Phe
  VARIANT 50: note = Xaa is Pro or Gln
  VARIANT 251: note = Xaa is Ser or Pro
  VARIANT 278: note = Xaa is Pro or Leu
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 68 - protein, 284 aa, synthetic construct
  REGION 1..284: note = synthetic sequence
  VARIANT 13: note = Xaa is Ser or Phe
  VARIANT 27: note = Xaa is Tyr or His
  VARIANT 31: note = Xaa is Thr, Ser, or Met
  VARIANT 34: note = Xaa is Leu or Val
  VARIANT 54: note = Xaa is Gln or Arg
  VARIANT 81: note = Xaa is Pro or Leu
  VARIANT 100: note = Xaa is Gly or Asp
  VARIANT 104: note = Xaa is Gly or Val
  VARIANT 105: note = Xaa is Ser or Cys
  VARIANT 110: note = Xaa is Leu or Ile
  VARIANT 159: note = Xaa is Tyr or His
  VARIANT 169: note = Xaa is His or Arg
  VARIANT 171: note = Xaa is Tyr or His
  VARIANT 178: note = Xaa is Met or Thr
  VARIANT 185: note = Xaa is Pro or Ala
  VARIANT 219: note = Xaa is Arg, Trp, or Gln
  VARIANT 258: note = Xaa is Thr or Met
  VARIANT 275: note = Xaa is Lys or Glu
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 69 - protein, 946 aa, synthetic construct
  REGION 1..946: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 70 - protein, 966 aa, synthetic construct
  REGION 1..966: note = synthetic sequence
VVVGAVGVGKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 71 - protein, 966 aa, synthetic construct
  REGION 1..966: note = synthetic sequence
VVVGAVGVGKGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 72 - protein, 934 aa, synthetic construct
  REGION 1..934: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 73 - protein, 793 aa, synthetic construct
  REGION 1..793: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 74 - protein, 781 aa, synthetic construct
  REGION 1..781: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 75 - protein, 939 aa, synthetic construct
  REGION 1..939: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 76 - protein, 943 aa, synthetic construct
  REGION 1..943: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAEAAAKEAAAKAAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 77 - protein, 946 aa, synthetic construct
  REGION 1..946: note = synthetic sequence
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 78 - protein, 946 aa, synthetic construct
  REGION 1..946: note = synthetic sequence
VVVGAVGVGKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 79 - protein, 946 aa, synthetic construct
  REGION 1..946: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSVVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 966 aa, synthetic construct
  REGION 1..966: note = synthetic sequence
VVVGAVGVGKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 81 - protein, 665 aa, synthetic construct
  REGION 1..665: note = synthetic sequence
VVVGAVGVGKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 935 aa, synthetic construct
  REGION 1..935: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 83 - protein, 943 aa, synthetic construct
  REGION 1..943: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 84 - protein, 947 aa, synthetic construct
  REGION 1..947: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 85 - protein, 951 aa, synthetic construct
  REGION 1..951: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPAPAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 939 aa, synthetic construct
  REGION 1..939: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPKPKPKPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 939 aa, synthetic construct
  REGION 1..939: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPEPEPEPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 88 - protein, 927 aa, synthetic construct
  REGION 1..927: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 941 aa, synthetic construct
  REGION 1..941: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGPGGGGPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 90 - protein, 946 aa, synthetic construct
  REGION 1..946: note = synthetic sequence
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGPGGGGPGGGGPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 91 - protein, 932 aa, synthetic construct
  REGION 1..932: note = synthetic sequence
  VARIANT 646: note = Xaa can be any naturally occurring amino acid
KLVVVGADGVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGXAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 92 - protein, 932 aa, synthetic construct
  REGION 1..932: note = synthetic sequence
  VARIANT 646: note = Xaa can be any naturally occurring amino acid
KLVVVGADGVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGXAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 93 - protein, 932 aa, synthetic construct
  REGION 1..932: note = synthetic sequence
  VARIANT 646: note = Xaa can be any naturally occurring amino acid
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGXAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 931 aa, synthetic construct
  REGION 1..931: note = synthetic sequence
  VARIANT 645: note = Xaa can be any naturally occurring amino acid
VVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGXAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, Homo sapiens [sequence omitted]

SEQ ID NO: 96 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 97 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 98 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 99 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 100 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 101 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 102 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 103 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 104 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 105 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 106 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 107 - protein, 188 aa, unidentified
  REGION 1..188: note = A KRAS polypeptide.
  VARIANT 5: note = Xaa can be Lys, Phe, or Leu
  VARIANT 7: note = Xaa can be Val or Leu
  VARIANT 8: note = Xaa can be Val or Thr
  VARIANT 9: note = Xaa can be Val or Thr
  VARIANT 12: note = Xaa can be Gly, Asp, Cys, Val, or Ser
  VARIANT 13: note = Xaa can be Gly, Cys, or Asp
MTEYXLXXXGAXXVGKSALTIQLIQNHFVDEYDPTIEDSYRKQVVIDGETCLWDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 108 - protein, 9 aa, Homo sapiens
VVGADGVGK

SEQ ID NO: 109 - protein, 9 aa, Homo sapiens
VVGACGVGK

SEQ ID NO: 110 - protein, 9 aa, Homo sapiens
VVGAVGVGK

SEQ ID NO: 111 - protein, 10 aa, Homo sapiens
VVVGADGVGK

SEQ ID NO: 112 - protein, 10 aa, Homo sapiens
VVVGAVGVGK

SEQ ID NO: 113 - protein, 10 aa, Homo sapiens
VVVGACGVGK

SEQ ID NO: 114 - protein, 9 aa, Homo sapiens
VTGADGVGK

SEQ ID NO: 115 - protein, 9 aa, Homo sapiens
VTGAVGVGK

SEQ ID NO: 116 - protein, 9 aa, Homo sapiens
VTGACGVGK

SEQ ID NO: 117 - protein, 10 aa, Homo sapiens
VTVGADGVGK

SEQ ID NO: 118 - protein, 10 aa, Homo sapiens
VTVGAVGVGK

SEQ ID NO: 119 - protein, 10 aa, Homo sapiens
VTVGACGVGK

SEQ ID NO: 120 - protein, 10 aa, Homo sapiens
VVVGAGDVGK

SEQ ID NO: 121 - protein, 9 aa, Homo sapiens
VVGAGDVGK

SEQ ID NO: 122 - protein, 10 aa, Homo sapiens
VVVGARGVGK

SEQ ID NO: 123 - protein, 9 aa, Homo sapiens
VVGARGVGK

SEQ ID NO: 124 - protein, 9 aa, Homo sapiens
LVVVGADGV

SEQ ID NO: 125 - protein, 9 aa, Homo sapiens
LVVVGAVGV

SEQ ID NO: 126 - protein, 9 aa, Homo sapiens
LVVVGACGV

SEQ ID NO: 127 - protein, 10 aa, Homo sapiens
KLVVVGADGV

SEQ ID NO: 128 - protein, 10 aa, Homo sapiens
KLVVVGAVGV

SEQ ID NO: 129 - protein, 10 aa, Homo sapiens
KLVVVGACGV

SEQ ID NO: 130 - protein, 9 aa, Homo sapiens
LLVVGADGV

SEQ ID NO: 131 - protein, 9 aa, Homo sapiens
LLVVGAVGV

SEQ ID NO: 132 - protein, 9 aa, Homo sapiens
LLVVGACGV

SEQ ID NO: 133 - protein, 10 aa, Homo sapiens
FLVVVGADGV

SEQ ID NO: 134 - protein, 10 aa, Homo sapiens
FLVVVGAVGV

SEQ ID NO: 135 - protein, 10 aa, Homo sapiens
FLVVVGACGV

SEQ ID NO: 136 - protein, 10 aa, Homo sapiens
KLVVVGAGDV

SEQ ID NO: 137 - protein, 10 aa, Homo sapiens
KLVVVGARGV

SEQ ID NO: 138 - protein, 10 aa, Homo sapiens
GAGDVGKSAL

SEQ ID NO: 139 - protein, 9 aa, Homo sapiens
AGDVGKSAL

SEQ ID NO: 140 - protein, 9 aa, Homo sapiens
DVGKSALTI

SEQ ID NO: 141 - protein, 10 aa, Homo sapiens
GAVGVGKSAL

SEQ ID NO: 142 - protein, 9 aa, Homo sapiens
AVGVGKSAL

SEQ ID NO: 143 - protein, 9 aa, Homo sapiens
YKLVVVGAV

SEQ ID NO: 144 - protein, 9 aa, Homo sapiens
ARGVGKSAL

SEQ ID NO: 145 - protein, 10 aa, Homo sapiens
GARGVGKSAL

SEQ ID NO: 146 - protein, 10 aa, Homo sapiens
EYKLVVVGAR

SEQ ID NO: 147 - protein, 10 aa, Homo sapiens
RGVGKSALTI

SEQ ID NO: 148 - protein, 9 aa, Homo sapiens
LVVVGARGV

SEQ ID NO: 149 - protein, 10 aa, Homo sapiens
GADGVGKSAL

SEQ ID NO: 150 - protein, 9 aa, Homo sapiens
ACGVGKSAL

SEQ ID NO: 151 - protein, 10 aa, Homo sapiens
GACGVGKSAL

SEQ ID NO: 152 - protein, 9 aa, Homo sapiens
VGAVGVGKS

SEQ ID NO: 153 - protein, 10 aa, Homo sapiens
VGAVGVGKSA

SEQ ID NO: 154 - protein, 10 aa, Homo sapiens
AVGVGKSALT

SEQ ID NO: 155 - protein, 9 aa, Homo sapiens
GAVGVGKSA

SEQ ID NO: 156 - protein, 10 aa, Homo sapiens
LVVVGAVGVG

SEQ ID NO: 157 - protein, 9 aa, Homo sapiens
KLVVVGAVG

SEQ ID NO: 158 - protein, 5 aa, synthetic construct
  REGION 1..5: note = synthetic sequence
GTLRG

SEQ ID NO: 159 - protein, 5 aa, synthetic construct
  REGION 1..5: note = synthetic sequence
YNQSE

SEQ ID NO: 160 - protein, 5 aa, synthetic construct
  REGION 1..5: note = synthetic sequence
TAADM

SEQ ID NO: 161 - protein, 5 aa, synthetic construct
  REGION 1..5: note = synthetic sequence
AQTTK

SEQ ID NO: 162 - protein, 5 aa, synthetic construct
  REGION 1..5: note = synthetic sequence
VETRP

SEQ ID NO: 163 - protein, 5 aa, synthetic construct
  REGION 1..5: note = synthetic sequence
GDGTF

SEQ ID NO: 164 - protein, 5 aa, synthetic construct
  REGION 1..5: note = synthetic sequence
RNLRG

SEQ ID NO: 165 - protein, 5 aa, synthetic construct
  REGION 1..5: note = synthetic sequence
TAADT

SEQ ID NO: 166 - protein, 5 aa, synthetic construct
  REGION 1..5: note = synthetic sequence
AQITQ

SEQ ID NO: 167 - protein, 5 aa, synthetic construct
  REGION 1..5: note = synthetic sequence
GDRTF

SEQ ID NO: 168 - protein, 133 aa, synthetic construct
  REGION 1..133: note = synthetic sequence
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 169 - protein, 133 aa, synthetic construct
  REGION 1..133: note = synthetic sequence
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 170 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 171 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 172 - protein, 240 aa, Homo sapiens
QEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSIL

SEQ ID NO: 173 - protein, 134 aa, Homo sapiens
LQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 174 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 175 - protein, 208 aa, Homo sapiens [sequence omitted]

SEQ ID NO: 176 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 177 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 178 - protein, 223 aa, synthetic construct
  REGION 1..223: note = synthetic sequence
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 179 - protein, 5 aa, synthetic construct
  REGION 1..5: note = synthetic sequence
GSGGS

SEQ ID NO: 180 - protein, 5 aa, synthetic construct
  REGION 1..5: note = synthetic sequence
GGGGS

SEQ ID NO: 181 - protein, 4 aa, synthetic construct
  REGION 1..4: note = synthetic sequence
GGGS

SEQ ID NO: 182 - protein, 4 aa, synthetic construct
  REGION 1..4: note = synthetic sequence
GGSG

SEQ ID NO: 183 - protein, 5 aa, synthetic construct
  REGION 1..5: note = synthetic sequence
GGSGG

SEQ ID NO: 184 - protein, 5 aa, synthetic construct
  REGION 1..5: note = synthetic sequence
GSGSG

SEQ ID NO: 185 - protein, 5 aa, synthetic construct
  REGION 1..5: note = synthetic sequence
GSGGG

SEQ ID NO: 186 - protein, 5 aa, synthetic construct
  REGION 1..5: note = synthetic sequence
GGGSG

SEQ ID NO: 187 - protein, 5 aa, synthetic construct
  REGION 1..5: note = synthetic sequence
GSSSG

SEQ ID NO: 188 - protein, 10 aa, synthetic construct
  REGION 1..10: note = synthetic sequence
GGGGSGGGGS

SEQ ID NO: 189 - protein, 15 aa, synthetic construct
  REGION 1..15: note = synthetic sequence
GGGGSGGGGSGGGGS

SEQ ID NO: 190 - protein, 20 aa, synthetic construct
  REGION 1..20: note = synthetic sequence
GGGGSGGGGSGGGGSGGGGS

SEQ ID NO: 191 - protein, 35 aa, synthetic construct
  REGION 1..35: note = synthetic sequence
GGGGSGGGGSGGGGSGGGGSGGGGSGGGGSGGGGS

SEQ ID NO: 192 - protein, 5 aa, synthetic construct
  REGION 1..5: note = synthetic sequence
AAAGG

SEQ ID NO: 193 - protein, 5 aa, synthetic construct
  REGION 1..5: note = synthetic sequence
  VARIANT 4: note = Xaa can be any naturally occurring amino acid
VPGXG

SEQ ID NO: 194 - protein, 5 aa, synthetic construct
  REGION 1..5: note = synthetic sequence
EAAAK

SEQ ID NO: 195 - protein, 7 aa, synthetic construct
  REGION 1..7: note = synthetic sequence
AEAAAKA

SEQ ID NO: 196 - protein, 16 aa, synthetic construct
  REGION 1..16: note = synthetic sequence
AEAAAKALEAEAAAKA

SEQ ID NO: 197 - protein, 10 aa, synthetic construct
  REGION 1..10: note = synthetic sequence
EAAAKEAAAK

SEQ ID NO: 198 - protein, 15 aa, synthetic construct
  REGION 1..15: note = synthetic sequence
EAAAKEAAAKEAAAK

SEQ ID NO: 199 - protein, 46 aa, synthetic construct
  REGION 1..46: note = synthetic sequence
AEAAAKEAAAKEAAAKEAAAKALEAEAAAKEAAAKEAAAKEAAAKA

SEQ ID NO: 200 - protein, 12 aa, synthetic construct
  REGION 1..12: note = synthetic sequence
  VARIANT 2..11: note = Each copy of Glu Ala Ala Ala Lys at positions 1 to 12 is either present or absent such that the number of copies of Glu Ala Ala Ala Lys ranges from 1 to 2.
AEAAAKEAAAKA

SEQ ID NO: 201 - protein, 5 aa, synthetic construct
  REGION 1..5: note = synthetic sequence
PAPAP

SEQ ID NO: 202 - protein, 8 aa, synthetic construct
  REGION 1..8: note = synthetic sequence
APAPAPAP

SEQ ID NO: 203 - protein, 12 aa, synthetic construct
  REGION 1..12: note = synthetic sequence
  VARIANT 1..12: note = Each copy of Ala Pro at positions 1 to 12 is either present or absent such that the number of copies of Ala Pro ranges from 2 to 6.
APAPAPAPAPAP

SEQ ID NO: 204 - protein, 16 aa, synthetic construct
  REGION 1..16: note = synthetic sequence
APAPAPAPAPAPAPAP

SEQ ID NO: 205 - protein, 20 aa, synthetic construct
  REGION 1..20: note = synthetic sequence
APAPAPAPAPAPAPAPAPAP

SEQ ID NO: 206 - protein, 4 aa, synthetic construct
  REGION 1..4: note = synthetic sequence
KPKP

SEQ ID NO: 207 - protein, 8 aa, synthetic construct
  REGION 1..8: note = synthetic sequence
KPKPKPKP

SEQ ID NO: 208 - protein, 12 aa, synthetic construct
  REGION 1..12: note = synthetic sequence
  VARIANT 1..12: note = Each copy of Lys Pro at positions 1 to 12 is either present or absent such that the number of copies of Lys Pro ranges from 2 to 6.
KPKPKPKPKPKP

SEQ ID NO: 209 - protein, 16 aa, synthetic construct
  REGION 1..16: note = synthetic sequence
KPKPKPKPKPKPKPKP

SEQ ID NO: 210 - protein, 20 aa, synthetic construct
  REGION 1..20: note = synthetic sequence
KPKPKPKPKPKPKPKPKPKP

SEQ ID NO: 211 - protein, 4 aa, synthetic construct
  REGION 1..4: note = synthetic sequence
EPEP

SEQ ID NO: 212 - protein, 8 aa, synthetic construct
  REGION 1..8: note = synthetic sequence
EPEPEPEP

SEQ ID NO: 213 - protein, 12 aa, synthetic construct
  REGION 1..12: note = synthetic sequence
  VARIANT 1..12: note = Each copy of Glu Pro at positions 1 to 12 is either present or absent such that the number of copies of Glu Pro ranges from 2 to 6.
EPEPEPEPEPEP

SEQ ID NO: 214 - protein, 16 aa, synthetic construct
  REGION 1..16: note = synthetic sequence
EPEPEPEPEPEPEPEP

SEQ ID NO: 215 - protein, 20 aa, synthetic construct
  REGION 1..20: note = synthetic sequence
EPEPEPEPEPEPEPEPEPEP

SEQ ID NO: 216 - protein, 5 aa, synthetic construct
  REGION 1..5: note = synthetic sequence
GCGGS

SEQ ID NO: 217 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
GCGGSGGGGSGGGGSGGGGSGGGGSGGGGSGGGGSGGGGSGGGGSGGGGSGGGGS

SEQ ID NO: 218 - protein, 15 aa, synthetic construct
  REGION 1..15: note = synthetic sequence
GCGGSGGGGSGGGGS

SEQ ID NO: 219 - protein, 5 aa, synthetic construct
  REGION 1..5: note = synthetic sequence
CGGGS

SEQ ID NO: 220 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
CGGGSGGGGSGGGGSGGGGSGGGGSGGGGSGGGGSGGGGSGGGGSGGGGSGGGGS

SEQ ID NO: 221 - protein, 5 aa, synthetic construct
  REGION 1..5: note = synthetic sequence
GGCGS

SEQ ID NO: 222 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
GGCGSGGGGSGGGGSGGGGSGGGGSGGGGSGGGGSGGGGSGGGGSGGGGSGGGGS

SEQ ID NO: 223 - protein, 5 aa, synthetic construct
  REGION 1..5: note = synthetic sequence
GGGCS

SEQ ID NO: 224 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
GGGCSGGGGSGGGGSGGGGSGGGGSGGGGSGGGGSGGGGSGGGGSGGGGSGGGGS

SEQ ID NO: 225 - protein, 5 aa, synthetic construct
  REGION 1..5: note = synthetic sequence
GGGGC

SEQ ID NO: 226 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
GGGGCGGGGSGGGGSGGGGSGGGGSGGGGSGGGGSGGGGSGGGGSGGGGSGGGGS

SEQ ID NO: 227 - protein, 6 aa, synthetic construct
  REGION 1..6: note = synthetic sequence
AEAAAK

SEQ ID NO: 228 - protein, 55 aa, synthetic construct
  REGION 1..55: note = synthetic sequence
  VARIANT 1..50: note = Each copy of Gly Gly Gly Gly Ser at positions 1 to 50 is either present or absent such that the number of copies of Gly Gly Gly Gly Ser ranges from 1 to 10.
GGGGSGGGGSGGGGSGGGGSGGGGSGGGGSGGGGSGGGGSGGGGSGGGGSGCGGS

SEQ ID NO: 229 - protein, 10 aa, synthetic construct
  REGION 1..10: note = synthetic sequence
VVVGAGGVGK

SEQ ID NO: 230 - protein, 9 aa, synthetic construct
  REGION 1..9: note = synthetic sequence
GACGVGKSA

SEQ ID NO: 231 - protein, 9 aa, synthetic construct
  REGION 1..9: note = synthetic sequence
GADGVGKSA

SEQ ID NO: 232 - protein, 4 aa, synthetic construct
  REGION 1..4: note = synthetic sequence
APAP

SEQ ID NO: 233 - protein, 10 aa, synthetic construct
  REGION 1..10: note = synthetic sequence
GGGGPGGGGP

SEQ ID NO: 234 - protein, 15 aa, synthetic construct
  REGION 1..15: note = synthetic sequence
GGGGPGGGGPGGGGP

SEQ ID NO: 235 - protein, 50 aa, synthetic construct
  REGION 1..50: note = synthetic sequence
  VARIANT 1..50: note = Each copy of Gly Gly Gly Gly Ser at positions 1 to 50 is either present or absent such that the number of copies of Gly Gly Gly Gly Ser ranges from 1 to 10.
GGGGSGGGGSGGGGSGGGGSGGGGSGGGGSGGGGSGGGGSGGGGSGGGGS

SEQ ID NO: 236 - protein, 100 aa, synthetic construct
  REGION 1..100: note = synthetic sequence
  VARIANT 1..100: note = Each copy of Glu Ala Ala Ala Lys at positions 1 to 100 is either present or absent such that the number of copies of Glu Ala Ala Ala Lys ranges from 1 to 20.
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 237 - protein, 102 aa, synthetic construct
  REGION 1..102: note = synthetic sequence
  VARIANT 2..101: note = Each copy of Glu Ala Ala Ala Lys at positions 2 to 101 is either present or absent such that the number of copies of Glu Ala Ala Ala Lys ranges from 1 to 20.
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 238 - protein, 206 aa, synthetic construct
  REGION 1..206: note = synthetic sequence
  VARIANT 2..101: note = Each copy of Glu Ala Ala Ala Lys at positions 2 to 101 is either present or absent such that the number of copies of Glu Ala Ala Ala Lys ranges from 1 to 20.
  VARIANT 106..205: note = Each copy of Glu Ala Ala Ala Lys at positions 106 to 205 is either present or absent such that the number of copies of Glu Ala Ala Ala Lys ranges from 1 to 20.
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 239 - protein, 40 aa, synthetic construct
  REGION 1..40: note = synthetic sequence
  VARIANT 1..40: note = Each copy of Glu Pro at positions 1 to 40 is either present or absent such that the number of copies of Glu Pro ranges from 1 to 20.
EPEPEPEPEPEPEPEPEPEPEPEPEPEPEPEPEPEPEPEP

SEQ ID NO: 240 - protein, 60 aa, synthetic construct
  REGION 1..60: note = synthetic sequence
  VARIANT 1..60: note = Each copy of Thr Pro Arg at positions 1 to 60 is either present or absent such that the number of copies of Thr Pro Arg ranges from 1 to 20.
TPRTPRTPRTPRTPRTPRTPRTPRTPRTPRTPRTPRTPRTPRTPRTPRTPRTPRTPRTPR

SEQ ID NO: 241 - protein, 40 aa, synthetic construct
  REGION 1..40: note = synthetic sequence
  VARIANT 1..40: note = Each copy of Ala Pro at positions 1 to 40 is either present or absent such that the number of copies of Ala Pro ranges from 1 to 20.
APAPAPAPAPAPAPAPAPAPAPAPAPAPAPAPAPAPAPAP

SEQ ID NO: 242 - protein, 40 aa, synthetic construct
  REGION 1..40: note = synthetic sequence
  VARIANT 1..40: note = Each copy of Lys Pro at positions 1 to 40 is either present or absent such that the number of copies of Lys Pro ranges from 1 to 20.
KPKPKPKPKPKPKPKPKPKPKPKPKPKPKPKPKPKPKPKP

SEQ ID NO: 243 - skipped

SEQ ID NO: 244 - protein, 51 aa, synthetic construct
  REGION 1..51: note = synthetic sequence
  VARIANT 2..51: note = Each copy of Glu Ala Ala Ala Lys at positions 2 to 51 is either present or absent such that the number of copies of Glu Ala Ala Ala Lys ranges from 1 to 10.
AEAAAKEAAAKEAAAKEAAAKEAAAKEAAAKEAAAKEAAAKEAAAKEAAAK

SEQ ID NO: 245 - protein, 52 aa, synthetic construct
  REGION 1..52: note = synthetic sequence
  VARIANT 2..51: note = Each copy of Glu Ala Ala Ala Lys at positions 2 to 51 is either present or absent such that the number of copies of Glu Ala Ala Ala Lys ranges from 1 to 10.
AEAAAKEAAAKEAAAKEAAAKEAAAKEAAAKEAAAKEAAAKEAAAKEAAAKA

SEQ ID NO: 246 - protein, 20 aa, synthetic construct
  REGION 1..20: note = synthetic sequence
  VARIANT 1..20: note = Each copy of Ala Pro at positions 1 to 20 is either present or absent such that the number of copies of Ala Pro ranges from 1 to 10.
APAPAPAPAPAPAPAPAPAP

SEQ ID NO: 247 - protein, 20 aa, synthetic construct
  REGION 1..20: note = synthetic sequence
  VARIANT 1..20: note = Each copy of Glu Pro at positions 1 to 20 is either present or absent such that the number of copies of Glu Pro ranges from 1 to 10.
EPEPEPEPEPEPEPEPEPEP

SEQ ID NO: 248 - protein, 20 aa, synthetic construct
  REGION 1..20: note = synthetic sequence
  VARIANT 1..20: note = Each copy of Lys Pro at positions 1 to 20 is either present or absent such that the number of copies of Lys Pro ranges from 1 to 10.
KPKPKPKPKPKPKPKPKPKP

SEQ ID NO: 249 - protein, 20 aa, synthetic construct
  REGION 1..20: note = synthetic sequence
  VARIANT 6..20: note = Each copy of Gly Gly Gly Gly Ser at positions 6 to 20 is either present or absent such that the number of copies of Gly Gly Gly Gly Ser ranges from 1 to 3.
GCGGSGGGGSGGGGSGGGGS

SEQ ID NO: 250 - protein, 25 aa, synthetic construct
  REGION 1..25: note = synthetic sequence
  VARIANT 6..25: note = Each copy of Gly Gly Gly Gly Ser at positions 6 to 25 is either present or absent such that the number of copies of Gly Gly Gly Gly Ser ranges from 2 to 4.
CGGGSGGGGSGGGGSGGGGSGGGGS

SEQ ID NO: 251 - protein, 25 aa, synthetic construct
  REGION 1..25: note = synthetic sequence
  VARIANT 6..25: note = Each copy of Gly Gly Gly Gly Ser at positions 6 to 25 is either present or absent such that the number of copies of Gly Gly Gly Gly Ser ranges from 2 to 4.
GCGGSGGGGSGGGGSGGGGSGGGGS

SEQ ID NO: 252 - protein, 25 aa, synthetic construct
  REGION 1..25: note = synthetic sequence
  VARIANT 1..20: note = Each copy of Gly Gly Gly Gly Ser at positions 1 to 20 is either present or absent such that the number of copies of Gly Gly Gly Gly Ser ranges from 2 to 4.
GGGGSGGGGSGGGGSGGGGSGCGGS

SEQ ID NO: 253 - protein, 35 aa, synthetic construct; identical to SEQ ID NO: 191
  REGION 1..35: note = synthetic sequence
  VARIANT 1..35: note = Each copy of Gly Gly Gly Gly Ser at positions 1 to 35 is either present or absent such that the number of copies of Gly Gly Gly Gly Ser ranges from 2, 3, 4, or 7.

(19) United States
(12) Patent Application Publication
(10) Pub. No.: US 2024/0067700 A1
(43) Pub. Date: Feb. 29, 2024

(54) T-CELL MODULATORY POLYPEPTIDES AND METHODS OF USE THEREOF

(71) Applicant: Cue Biopharma, Inc., Boston, MA (US)

(72) Inventors: Ronald D. Seidel, III, Boston, MA (US); Rodolfo J. Chaparro, Cambridge, MA (US); John F. Ross, Boston, MA (US); Saso Cemerski, Boston, MA (US)

(21) Appl. No.: 18/234,640

(22) Filed: Aug. 16, 2023

Related U.S. Application Data
(63) Continuation of application No. PCT/US22/20819, filed on Mar. 17, 2022.
(60) Provisional application No. 63/163,270, filed on Mar. 19, 2021.

(51) Int. Cl. C07K 14/74; A61K 39/00; C07K 14/47; C07K 14/55

(52) U.S. Cl. CPC C07K 14/70539; A61K 39/001164; C07K 14/47; C07K 14/55; A61K 2039/55533; A61K 2039/605; A61K 2039/6056; A61K 2039/627; C07K 2319/30

(57) ABSTRACT

The present disclosure provides T-cell modulatory polypeptides (TMPs) that comprise a MOD, class I HLA polypeptides (a class I HLA heavy chain polypeptide and a β2 microglobulin polypeptide), and a KRAS peptide (e.g., a KRAS peptide comprising a cancer-associated mutation) that presents an epitope to a T-cell receptor. A TMP is useful for modulating the activity of a T cell, and for modulating an immune response in an individual.

### CROSS-REFERENCE

This application claims the benefit of U.S. Provisional Patent Application No. 63/163,270, filed Mar. 19, 2021, which application is incorporated herein by reference in its entirety.

### INCORPORATION BY REFERENCE OF SEQUENCE LISTING PROVIDED AS A TEXT FILE

A Sequence Listing is provided herewith as a text file. “CUEB-140WO_SeqListing_ST25.txt” created on Mar. 15, 2022 and having a size of 462 KB. The contents of the text file are incorporated by reference herein in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The costimulatory proteins on the APC also are referred to as “immunomodulatory” proteins because they modulate the activity of the T cell when they bind the costimulatory protein on the T cell, with the specific modulation being a function of which immunomodulatory protein on the APC binds to which costimulatory protein on the T cell. The TCR is specific for a given epitope; however, the T cell's costimulatory protein is not epitope-specific and instead is generally expressed on all T cells or on large T cell subsets.

### SUMMARY

The present disclosure provides single-chain T-cell modulatory polypeptides (TMPs), and dimers thereof, that comprise an immunomodulatory polypeptide (“MOD”), class I HLA polypeptides (a class I HLA heavy chain polypeptide and a β2 microglobulin polypeptide), and a KRAS peptide (e.g., a KRAS peptide comprising a cancer-associated mutation) that presents an epitope to a T-cell receptor. A TMP is useful for modulating the activity of a T cell, and for modulating an immune response in an individual.

### BRIEF DESCRIPTION OF THE DRAWINGS

FIG. 1A-1B provide an amino acid sequence of a wild-type human β2M polypeptide (FIG. 1A; SEQ ID NO:3) and an amino acid sequence of a β2M polypeptide with an R12C substitution (FIG. 1B; SEQ ID NO:4).

FIGS. 2A-2M provide amino acid sequences of immunoglobulin Fc polypeptides (SEQ ID NOs:5-17, respectively).

FIG. 3A-3E provide amino acid sequences of wild-type HLA-A*0201 (FIG. 3A; SEQ ID NO:18) and variants (FIG. 3B-3E; SEQ ID NOs:19-22, respectively).

FIG. 4A-4E provide amino acid sequences of wild-type HLA-A*1101 (FIG. 4A; SEQ ID NO:23) and variants (FIG. 4B-4E; SEQ ID NOs:24-27, respectively).

FIG. 5A-SE provide amino acid sequences of wild-type HLA-A*2402 (FIG. 5A; SEQ ID NO:28) and variants (FIG. 5B-5E; SEQ ID NOs:29-32, respectively).

FIG. 6A-6E provide amino acid sequences of wild-type HLA-A*3303 (FIG. 6A; SEQ ID NO:33) and variants (FIG. 6B-6E; SEQ ID NOs:34-37, respectively).

FIGS. 7A-7B provide an alignment of HLA-A heavy chain amino acid sequences (FIG. 7A; SEQ ID NO:38-46, respectively) and a consensus sequence (FIG. 7B; SEQ ID NO:47).

FIGS. 8A-8B provide an alignment of HLA-B heavy chain amino acid sequences (FIG. 8A; SEQ ID NOs:48-54, respectively) and a consensus sequence (FIG. 8B; SEQ ID NO:55).

FIGS. 9A-9B provide an alignment of HLA-C heavy chain amino acid sequences (FIG. 9A; SEQ ID NOs:56-64, respectively) and a consensus sequence (FIG. 9B; SEQ ID NO:65).

FIG. 10 provides a consensus amino acid sequence for each of HLA-E, -F, and -G heavy chains (SEQ ID NOs:66-68, respectively). Variable amino acid (aa) positions are indicated as “X” residues sequentially numbered; the locations of amino acids 84, 139, and 236 are double underlined.

FIG. 11 provides an alignment of consensus amino acid sequences for HLA-A (SEQ ID No:47), -B (SEQ ID NO:55), -C(SEQ ID NO:65), -E (SEQ ID NO:66), -F (SEQ ID NO:67), and -G (SEQ ID NO:68).

FIG. 12 provides schematic depictions of examples of positions of immunomodulatory polypeptides in “single-chain” TMPs of the present disclosure.

FIG. 13 provides a schematic depiction of disulfide-linked single-chain TMPs of the present disclosure.

FIG. 14A-14V provide amino acid sequences of some examples of single-chain TMPs of the present disclosure (SEQ ID NOs:69-90, respectively).

FIG. 15A-15E depict in vitro activity of some examples of single-chain TMPs of the present disclosure.

FIGS. 16A-16D provide amino acid sequences of some examples of single-chain TMPs of the present disclosure (SEQ ID NOs:91-94, respectively).

FIGS. 17A-17D provide amino acid sequences of a wild-type IL-2 polypeptide (FIG. 17A; SEQ ID NO:95), IL-2Rα (FIG. 17B; SEQ ID NO:96), IL-2Rβ (FIG. 17C; SEQ ID NO:97), and IL-2Rγ (FIG. 17D; SEQ ID NO:98).

FIG. 18A-18D provide amino acid sequences of HLA-E heavy chains (SEQ ID NOs:99-102, respectively).

FIG. 19A-19D provide amino acid sequences of HLA-G heavy chains (SEQ ID NOs:103-106, respectively).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990). J. Mol. Biol. 215:403-10. Unless otherwise stated, “sequence identity” as referred to herein is determined by BLAST (Basic Local Alignment Search Tool), as described in Altschul et al. ((1990) J. Mol. Biol. 215:403), using default parameters.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. As discussed herein, a MOD can include, but is not limited to wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40. CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically hinds with B7-H3. MOD of a TMP can bind a cognate costimulatory polypeptide (i.e., a “co-MOD”) that is present on a target T cell.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant.” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (Ku). As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g., with reference to binding of a TMP to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower Kn. “Specific binding” generally refers to binding of a ligand to a moiety that is than its designated binding site or receptor. “Non-specific binding” generally refers to binding of a ligand to a moiety other than its designated binding site or receptor. “Covalent binding” or “covalent bond.” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease. i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient.” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject.” “host,” and “patient,” refer to a human.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely does not induce cell lysis.

As used herein, the term “about” used in connection with an amount indicates that the amount can vary by 10% of the stated amount. For example. “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

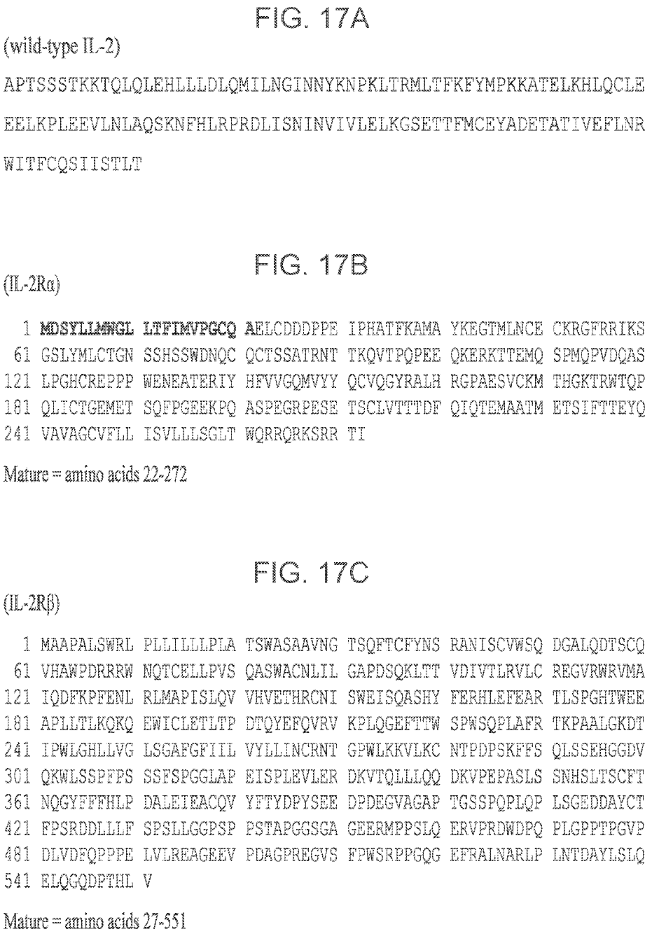

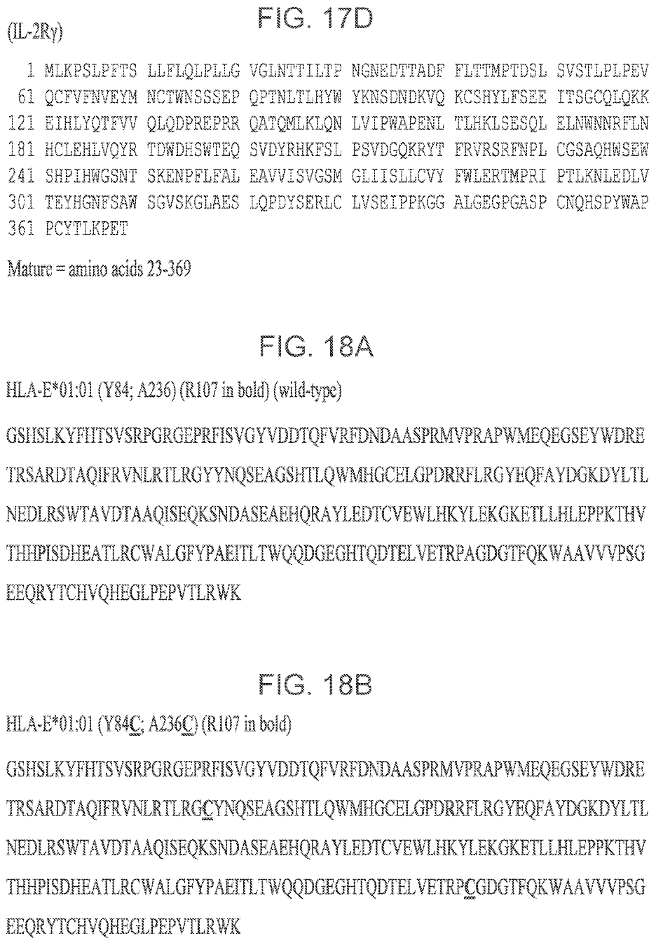

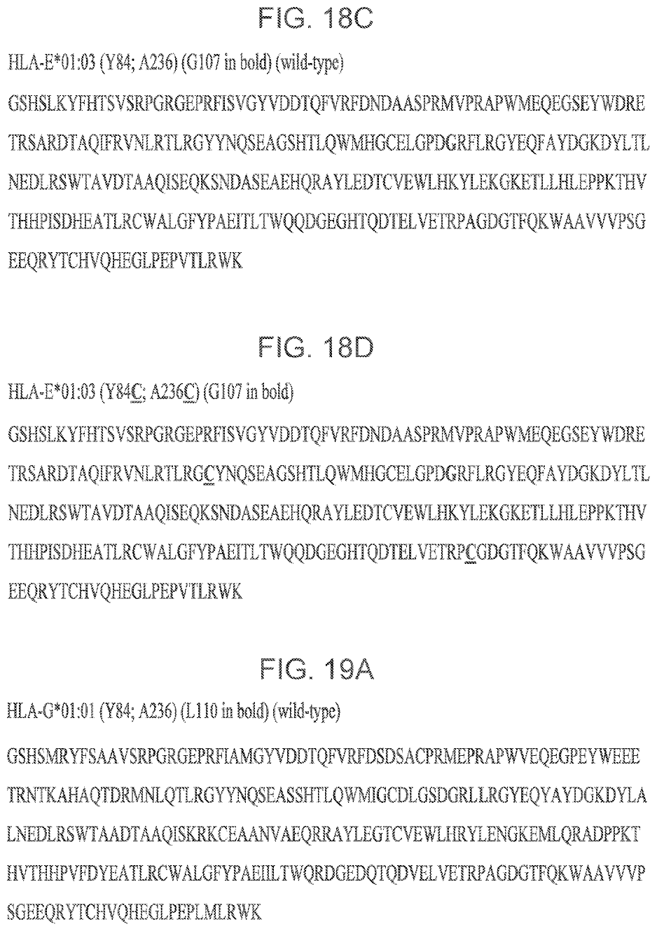

As used herein, the term “MHC heavy chain polypeptide” means collectively the domains of an MHC heavy chain polypeptide that are present in a TMP. For example, as illustrated in FIGS. 17 and 18, the MHC heavy chain polypeptide can comprise the α1, α2 and α3 domains.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a.” “an,” and “the” include plural referents unless the context clearly dictates otherwise. Thus, for example, reference to a “T-cell modulatory polypeptide” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides T-cell modulatory polypeptides that comprise a MOD and that comprise an epitope-presenting peptide. A TMP is useful for modulating the activity of a T cell, and for modulating an immune response in an individual.

### T-Cell Modulatory Polypeptides

The present disclosure provides a T-cell modulatory polypeptide (TMP), where the TMP is a single-chain polypeptide comprising: i) a KRAS peptide that, when bound to major histocompatibility complex (MHC) polypeptides, presents an epitope to a T-cell receptor (TCR); ii) a first MHC polypeptide; iii) a second MHC polypeptide; iv) one or more MODs; and optionally an immunoglobulin (Ig) Fc polypeptide or a non-Ig scaffold. As discussed below, single-chain TMPs can self-assemble into dimers, e.g., when the TMP comprises an Ig Fc, e.g., an IgG1 Fc. Disulfide bonds between Ig Fc polypeptides in two single-chain TMPs will spontaneously form to bond the two single-chain TMPs to form a homodimer.

A TMP of this disclosure includes at least one rigid peptide linker (discussed below) that is interposed between components of the TMP in order to reduce the potential for unfavorable interactions between different polypeptide domains within the TMP. For example, in some cases, a single-chain TMP comprises a rigid peptide linker between an Ig Fc polypeptide and a MOD. A rigid peptide linker also may be interposed between a first MOD and a second MOD. As discussed below, the presence of the rigid peptide linker may provide greater thermal stability as compared to a control TMP that does not comprise the rigid peptide linker.

Alternatively, or additionally, a TMP of this disclosure includes at least one short flexible peptide linker (discussed below) that is interposed between components of the TMP in order to reduce the potential for unfavorable interactions between different polypeptide domains within the TMP. For example, in some cases, a single-chain TMP comprises a short flexible peptide linker between an Ig Fc polypeptide and a MOD. A short flexible peptide linker also may be interposed between a first MOD and a second MOD. As discussed below, the presence of the short flexible peptide linker may provide greater thermal stability as compared to a control TMP that docs not comprise the rigid peptide linker.

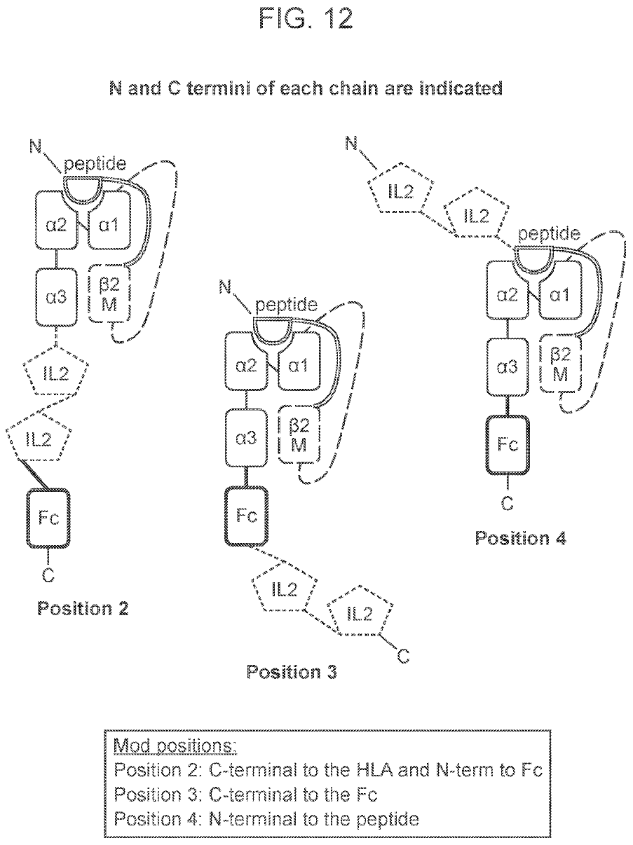

In some cases, a TMP comprises, in order from N-terminus to C-terminus: i) a KRAS peptide; ii) a first MHC polypeptide; iii) a second MHC polypeptide; iv) one or more MODs; and v) an Ig Fc polypeptide. In some cases, a TMP comprises, in order from N-terminus to C-terminus: i) a KRAS peptide; ii) a β2M polypeptide; iii) a class I MHC heavy chain polypeptide; iv) one or more MODs; and v) an Ig Fc polypeptide. As discussed below, peptide linkers may be interposed between two or more of the components. This arrangement of components is referred to as MOD Position 2 in FIG. 12.

In some cases, a TMP comprises, in order from N-terminus to C-terminus: i) a KRAS peptide; ii) a first MHC polypeptide; iii) a second MHC polypeptide; iv) an Ig Fc polypeptide; and v) one or more MODs. In some cases, a TMP comprises, in order from N-terminus to C-terminus: i) a KRAS peptide; ii) a β2M polypeptide; iii) a class I MHC heavy chain polypeptide; iv) an Ig Fc polypeptide; and v) one or more MODs. As discussed below, peptide linkers may be interposed between two or more of the components. This arrangement of components is referred to as MOD Position 3 in FIG. 12.

In some cases, a TMP comprises, in order from N-terminus to C-terminus: i) one or more MODs; ii) a KRAS peptide; iii) a first MHC polypeptide; iv) a second MHC polypeptide; and v) an Ig Fc polypeptide. In some cases, a TMP comprises, in order from N-terminus to C-terminus: i) one or more MODs; ii) a KRAS peptide; iii) a first class I MHC polypeptide; iv) a second class I MHC polypeptide; and v) an Ig Fc polypeptide. In some cases, a TMP comprises, in order from N-terminus to C-terminus: i) one or more MODs; ii) a KRAS peptide; iii) a β2M polypeptide; iv) a class I MHC heavy chain polypeptide; and v) an Ig Fc polypeptide. As discussed below, peptide linkers may be interposed between two or more of the components. This arrangement of components is referred to as MOD Position 4 in FIG. 12.

A MOD may comprise either a wild type (“wt”) immunomodulatory polypeptide or a variant of a wt immunomodulatory polypeptide. Where a MOD comprises a variant, it may exhibit reduced binding to its co-MOD, including e.g., reduced binding to one or more chains or domains of the co-MOD. In such cases, the combination of the reduced affinity of the MOD for its co-MOD, and the affinity of the KRAS peptide for a TCR, may provide for enhanced selectivity of a TMP.

Binding affinity between a MOD and its co-MOD can be determined by bio-layer interferometry (BLI) using purified MOD and purified co-MOD. Binding affinity between a MOD present in a TMP and its co-MOD can be determined by BLI using purified TMP and the co-MOD. BLI methods are well known to those skilled in the art. See, e.g., Lad et al. (2015) J. Biomol. Screen. 20(4):498-507; and Shah and Duncan (2014) J. Vis. Exp. 18:e51383.

Unless otherwise stated herein, the affinity of a MOD for a co-MOD, or the affinity of a MOD on a TMP for a co-MOD, is determined using BLI as described in in published PCT application WO 2020/132138, published Jun. 25, 2020. See. e.g., paragraphs [0056]-[0057].

### KRAS Peptides

A TMP of this disclosure comprises a KRAS peptide that presents a KRAS epitope to a T cell (i.e., to a TCR present on the surface of a T cell) when the KRAS peptide is present in an MHC/peptide complex (e.g., an HLA/peptide complex). As used herein, the term “KRAS peptide” means a peptide that presents a KRAS epitope to a TCR when the KRAS peptide is bound to an MHC complex. As used herein, the term “KRAS epitope” means an epitope found on a KRAS protein. As used herein, the terms “KRAS” and “KRAS protein” are synonymous and mean a protein having an amino acid sequence present in one of the following: (i) a KRAS4A polypeptide; (ii) a KRAS4B; and (iii) variants of (i) and (ii) that occur in a human cancer, including, e.g., mutated forms. As used herein, the term “KRAS polypeptide” means a polypeptide having a sequence of amino acids found in all or a part of a KRAS protein, or where specified, a polypeptide having at least 75%, at least 80%, at least 85%, at least 90%, at least 95%, at least 98%, at least 99%, or 100% amino acid sequence identity to a sequence of amino acids found in all or a part of a KRAS protein or a variant that occurs in a human cancer, including, e.g., mutated forms.

KRAS (also known as “KRAS proto-oncogene. GTPase.” Kirsten rat sarcoma viral oncogene homolog.” and “K-Ras P21 protein”) is a GTPase that controls cell proliferation. When mutated, KRAS can fail to control cell proliferation, leading to cancer.

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:1).

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK TSKEEKTPGC VKIKKCIIM (SEQ ID NO:2).

Mutated forms of KRAS are associated with certain cancers; and at least a portion of the mutated form of KRAS is present on the surface of certain cancer cells. See, e.g., Prior et al. (2012) Cancer Res. 72:2457; and Warren and Holt (2010) Human Immunology 71:245. In SEQ ID NO:1 and SEQ ID NO:2, amino acids G12, G13, T35, I36, E49, Q61, K127, and A156 are in bold and underlined; substitutions of one or more of these residues can be present in a cancer-associated form of a KRAS polypeptide. A cancer-associated KRAS polypeptide can include one or more of: i) a substitution of G12 (e.g. G12C, G12V, G125, G12A, G12R, G12F, or G12D); ii) a substitution of G13 (e.g. G13C, G13D, G13R, G13V, G13S, or G13A); iii) a substitution of T35 (e.g., T35I); iv) a substitution of 136 (e.g., I36L or I36M); v) a substitution of E49 (e.g., E49K); vi) a substitution of Q61 (e.g. Q61H, Q61R, Q61P, Q61E, Q61K, Q61 L, or Q61K); vii) a substitution of K117 (e.g., K117N); and viii) a substitution of A146 (e.g. A146T or A146V); where the amino acid numbering is as set out in SEQ ID NO:1 and SEQ ID NO:2. See, e.g., U.S. 2019/0194192.

For example, a cancer-associated, mutated form of a KRAS polypeptide can have one or more amino acid substitutions compared to the amino acid sequence set forth in SEQ ID NO:1 or SEQ ID NO:2. In some cases, a cancer-associated, mutated form of a KRAS polypeptide has only a single amino acid substitution compared to the amino acid sequence set forth in SEQ ID NO:1 or SEQ ID NO:2. In some cases, a cancer-associated, mutated form of a KRAS polypeptide has only two amino acid substitutions compared to the amino acid sequence set forth in SEQ ID NO:1 or SEQ ID NO:2. In some cases, a cancer-associated, mutated form of a KRAS polypeptide has only three amino acid substitutions compared to the amino acid sequence set forth in SEQ ID NO:1 or SEQ ID NO:2. In some cases, a cancer-associated, mutated form of a KRAS polypeptide has only four amino acid substitutions compared to the amino acid sequence set forth in SEQ ID NO:1 or SEQ ID NO:2. In some cases, a cancer-associated, mutated form of a KRAS polypeptide has only five amino acid substitutions compared to the amino acid sequence set forth in SEQ ID NO:1 or SEQ ID NO:2.

For example, KRAS(G12D) (a KRAS polypeptide having a G-to-D substitution at amino acid position 12, based on the amino acid numbering set forth in SEQ ID NO:1) is associated with pancreatic ductal adenocarcinoma (PDAC). KRAS(G12V) (a KRAS polypeptide having a G-to-V substitution at amino acid position 12, based on the amino acid numbering set forth in SEQ ID NO:1 or SEQ ID NO:2) is also associated with pancreatic cancer. KRAS(G12R) (a KRAS polypeptide having a G-to-R substitution at amino acid position 12, based on the amino acid numbering set forth in SEQ ID NO:1 or SEQ ID NO:2) is also associated with pancreatic cancer. See, e.g., Waters and Der (2018) Cold Spring Harb. Perspect. Med. 8:(9), pii: a031435. doi: 10.1101/cshperspect.a031435. As another example, KRAS(G12C) (a KRAS polypeptide having a G-to-C substitution at amino acid position 12, based on the amino acid numbering set forth in SEQ ID NO:1 or SEQ ID NO:2) is associated with lung cancer, e.g., non-small cell lung cancer. Sec. e.g., Román et al. (2018) Mol. Cancer 17:33. Other mutated forms of KRAS (e.g., G12A; G12C; G12D; G12R; G12S; G12V; G13A; G13C; G13D; G13R; G13S; G13V) are associated with various cancers; where such cancers include, e.g., bile duct carcinoma, gall bladder carcinoma, adenocarcinoma, rectal adenocarcinoma, endometrial carcinoma, hematopoietic neoplasms, and lung cancer. See. e.g., Prior et al. (20120 Cancer Res. 72:2457.

As another example, a cancer-associated, mutated form of a KRAS polypeptide can have an amino acid substitution at amino acid 61 of a KRAS polypeptide (e.g., a KRAS polypeptide having the amino acid sequence set forth in SEQ ID NO:1 or SEQ ID NO:2). For example, a cancer-associated, mutated form of a KRAS polypeptide can have an amino acid substitution such as Q61H, Q61L, Q61E, Q61R, or Q61K.

Generally speaking, the KRAS peptide/MHC complex present in a TMP of the present disclosure hinds to a T-cell receptor (TCR) on a T cell with an affinity of at least 100 μM (e.g., at least 10 μM, at least 1 μM, at least 100 nM, at least 10 nM, or at least 1 nM). Further, generally speaking, a TMP binds to a T cell having a co-MOD and a TCR that binds the KRAS peptide/MHC complex of the TMP with an affinity that is greater (e.g., 25% greater) than the affinity with which the same TMP binds a second T cell that has the same co-MOD but has a TCR that substantially does not bind the KRAS peptide/MHC complex, e.g., the KRAS peptide/MHC complex binds to the TCR with an affinity less than 10⁻⁷M.

A KRAS epitope present in a TMP is a peptide specifically bound by a T-cell, i.e., the epitope is specifically bound by an epitope-specific T cell, i.e., a T cell having a TCR that is specific for the KRAS epitope. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

In some cases, a suitable KRAS peptide is a peptide of at least 4 amino acids in length, e.g., from 4 amino acids to about 20 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa and 20 aa, including a range of from 9-11 amino acids, from 6 to 15 amino acids, from 8 to 12 amino acids, from 5 to 10 amino acids, from 10 to 15 amino acids, and from 15 to 20 amino acids in length) of a KRAS polypeptide comprising an amino acid sequence having at least 80%, at least 85%, at least 90%, at least 95%, at least 98%, or at least 99%, amino acid sequence identity to one of the following KRAS amino acid sequences:
(A) MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:1), where the KRAS polypeptide comprises one or more (e.g., 1, 2, 3, 4, or 5) amino acid substitutions compared to the amino acid sequence forth in SEQ ID NO:1, and where the one or more amino acid substitutions can include substitutions associated with cancer; e.g., substitutions that are found in a KRAS polypeptide in a cancer cell;
(B) MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:2), where the KRAS polypeptide comprises one or more (e.g., 1, 2, 3, 4, or 5) amino acid substitutions compared to the amino acid sequence forth in SEQ ID NO:1, and where the one or more amino acid substitutions can include substitutions associated with cancer; e.g., substitutions that are found in a KRAS polypeptide in a cancer cell; and
(C) MTEY(X1)L(X2)(X3)(X4)GA(X5)(X6)VGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:107), where X1 is Lys, Phe, or Leu; X2 is Val or Leu; X3 is Val or Thr; X4 is Val or Thr; X5 is Gly, Asp, Cys, Val, or Ser; and X6 is Gly, Cys, or Asp; where one or both of X5 and X6 is not a Cys.

Non-limiting examples of suitable KRAS peptides include peptides comprising a sequence selected from the group consisting of VVGADGVGK (SEQ ID NO:108), VVGACGVGK (SEQ ID NO:109), VVGAVGVGK (SEQ ID NO:110), VVVGADGVGK (SEQ ID NO:11), VVVGAVGVGK (SEQ ID NO: 112). VVVGACGVGK (SEQ ID NO:113), VTGADGVGK (SEQ ID NO:114), VTGAVGVGK (SEQ ID NO:115), VTGACGVGK (SEQ ID NO:116), VTVGADGVGK (SEQ ID NO:117), VTVGAVGVGK (SEQ ID NO:118), and VTVGACGVGK (SEQ ID NO:119); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of: VVVGAGDVGK (SEQ ID NO:120); VVGAGDVGK (SEQ ID NO:121); VVVGARGVGK (SEQ ID NO:122); and VVGARGVGK (SEQ ID NO:123); where the KRAS peptide has a length of 9 amino acids or 10 amino acids, or a length of at least 9 amino acids or 10 amino acids.

Non-limiting examples of suitable KRAS peptides include peptides comprising a sequence selected from the group consisting of LVVVGADGV (SEQ ID NO:124), LVVVGAVGV (SEQ ID NO:125), LVVVGACGV (SEQ ID NO:126), KLVVVGADGV (SEQ ID NO:127), KLVVVGAVGV (SEQ ID NO:128), KLVVVGACGV (SEQ ID NO:129), LLVVGADGV (SEQ ID NO:130), LLVVGAVGV (SEQ ID NO:131), LLVVGACGV (SEQ ID NO:132), FLVVVGADGV (SEQ ID NO:133). FLVVVGAVGV (SEQ ID NO:135), and FLVVVGACGV (SEQ ID NO:135); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of: KLVVVGAGDV (SEQ ID NO:136); and KLVVVGARGV (SEQ ID NO:137); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of: GAGDVGKSAL (SEQ ID NO:138); AGDVGKSAL (SEQ ID NO:139); DVGKSALTI (SEQ ID NO:140); GAVGVGKSAL (SEQ ID NO:141); AVGVGKSAL (SEQ ID NO:142); YKLVVVGAV (SEQ ID NO:143); ARGVGKSAL (SEQ ID NO:144); GARGVGKSAL (SEQ ID NO:145); EYKLVVVGAR (SEQ ID NO:146); RGVGKSALTI (SEQ ID NO:147); LVVVGARGV (SEQ ID NO:148); GADGVGKSAL (SEQ ID NO:149); ACGVGKSAL (SEQ ID NO:150); and GACGVGKSAL (SEQ ID NO:151); where the KRAS peptide has a length of 9 amino acids or 10 amino acids, or a length of at least 9 amino acids or 10 amino acids.

Additional non-limiting examples of suitable KRAS peptides include peptides comprising a sequence selected from the group consisting of VVVGAGGVGK (SEQ ID NO:229); VVVGACGVGK (SEQ ID NO:113); VVVGADGVGK (SEQ ID NO:111); VVVGARGVGK (SEQ ID NO:122); VVVGAVGVGK (SEQ ID NO: 112); and VVGAVGVGK (SEQ ID NO:110), where the KRAS peptide has a length of 9 amino acids or 10 amino acids, or a length of at least 9 amino acids or 10 amino acids. Such peptides may present an epitope when bound to an HLA complex comprising a β2M polypeptide and an A*03:01 HLA-A heavy chain. Such peptides may present an epitope when bound to an HLA complex comprising a β2M polypeptide and an HLA A*11:01 heavy chain. In some cases, a peptide such as VVVGARGVGK (SEQ ID NO:122), VVGARGVGK (SEQ ID NO:123), VVVGAVGVGK (SEQ ID NO:112), or VVGAVGVGK (SEQ ID NO:110) and having a length of 9 or 10 amino acids will present an epitope when bound to an HLA complex comprising a β2M polypeptide and an A*30:01 HLA-A heavy chain. In some cases, a peptide such as VVVGACGVGK (SEQ ID NO:113). VVVGADGVGK (SEQ ID NO:111), VVVGARGVGK (SEQ ID NO:122), VVVGAVGVGK (SEQ ID NO: 112), or VVGAVGVGK (SEQ ID NO: 110), and having a length of 9 or 10 amino acids will present an epitope when bound to an HLA complex comprising a β2M polypeptide and an A*68:01 HLA heavy chain.

Additional non-limiting examples of suitable KRAS peptides include peptides comprising the amino acid sequence GAIDGVGKSAL (SEQ ID NO:149) or GARGVGKSAL (SEQ ID NO:145), where the KRAS peptide has a length of 9 amino acids or 10 amino acids, or a length of at least 9 amino acids or 10 amino acids. Such peptides may present an epitope when bound to an HLA complex comprising a β2M polypeptide and a B*07:02 HLA-B heavy chain.

Additional non-limiting examples of suitable KRAS peptides include peptides comprising a sequence selected from the group consisting of AVGVGKSAL (SEQ ID NO:142), GAVGVGKSAL (SEQ ID NO:141), GAVGVGKSA (SEQ ID NO:155), GACGVGKSA (SEQ ID NO:230), GADGVGKSAL (SEQ ID NO:149), and GADGVGKSA (SEQ ID NO:231), where the KRAS peptide has a length of 9 amino acids or 10 amino acids, or a length of at least 9 amino acids or 10 amino acids. In some cases, a peptide such as AVGVGKSAL (SEQ ID NO:142) and having a length of 9 amino acids will present an epitope when bound to an HLA complex comprising a β2M polypeptide and a C*01:02 HLA-C heavy chain. In some cases, a peptide such as GAVGVGKSAL (SEQ ID NO:141) and having a length of 10 amino acids, or a peptide such as GAVGVGKSA (SEQ ID NO:155) and having a length of 9 amino acids, will present an epitope when bound to an HLA complex comprising a β2M polypeptide and a C*03:03 HLA-C heavy chain. In some cases, a peptide such as GACGVGKSA (SEQ ID NO:230). GADGVGKSAL (SEQ ID NO:149), GAVGVGKSAL (SEQ ID NO:141), or GAVGVGKSA (SEQ ID NO:155), and having a length of 9 amino acids or 10 amino acids, will present an epitope when bound to an HLA complex comprising a β2M polypeptide and a C*03:04 HLA-C heavy chain. In some cases, a peptide such as GADGVGKSAL (SEQ ID NO:149) and having a length of 10 amino acids, or a peptide such as GADGVGKSA (SEQ ID NO:231) and having a length of 9 amino acids, will present an epitope when bound to an HLA complex comprising a β2M polypeptide and a C*08:02 HLA-C heavy chain.

In some cases, a TMP modulates the activity of a T cell that comprises a TCR that is specific for a G12V form of a KRAS polypeptide, as described above. In such cases, the KRAS peptide present in a TMP can comprise, e.g., one of the following amino acid sequences: VVGAVGVGK (SEQ ID NO: 110). VVVGAVGVGK (SEQ ID NO: 112), VGAVGVGKS (SEQ ID NO:152), VGAVGVGKSA (SEQ ID NO:153), AVGVGKSAL (SEQ ID NO:142), AVGVGKSALT (SEQ ID NO:154), GAVGVGKSAL (SEQ ID NO:141), GAVGVGKSA (SEQ ID NO:155), LVVVGAVGVG (SEQ ID NO:156), LVVVGAVGV (SEQ ID NO:125), KLVVVGAVGV (SEQ ID NO:128), and KLVVVGAVG (SEQ ID NO:157); where the KRAS peptide has a length of 9 amino acids or 10 amino acids, or a length of at least 9 amino acids or 10 amino acids.

In some cases, the KR AS peptide present in a TMP presents an epitope specific to an HLA-A, -B, -C, -E, -F, or -G allele. In an embodiment, the KRAS peptide present in a TMP presents an epitope restricted to HLA-A*0101, A*0201, A*0203, A*0301, A*1101, A*2301, A*2402. A*2407, A*3101, A*3303, A*3401, and/or A*6801. In an embodiment, the KRAS epitope peptide present in a TMP presents an epitope restricted to HLA-B*0702, B*0801, B*1502, B*2705, B*3802, B*3802, B*3901. B*3902. B*4001, B*4601, B*5101, and/or B*5301. In an embodiment, the KRAS epitope peptide present in a TMP presents an epitope restricted to C*0102. C*0303, C*0304, C*0401, C*0602. C*0701, C*702. C*0801, and/or C*1502.

As non-limiting examples, KRAS peptides comprising a peptide selected from the group consisting of VVGADGVGK (SEQ ID NO: 108). VVGACGVGK (SEQ ID NO:109), VVGAVGVGK (SEQ ID NO: 110), VVVGADGVGK (SEQ ID NO:111). VVVGAVGVGK (SEQ ID NO: 112), VVVGACGVGK (SEQ ID NO: 113). VTGADGVGK (SEQ ID NO: 114). VTGAVGVGK (SEQ ID NO:115), VTGACGVGK (SEQ ID NO:116), VTVGADGVGK (SEQ ID NO:117), VTVGAVGVGK (SEQ ID NO:118), VTVGACGVGK (SEQ ID NO:119), VVVGAGDVGK (SEQ ID NO:120), VVGAGDVGK (SEQ ID NO:121), VVVGARGVGK (SEQ ID NO:122), and VVGARGVGK (SEQ ID NO:123), where the KRAS peptide has a length of 9 amino acids or 10 amino acids, or a length of at least 9 amino acids or 10 amino acids, present an epitope when bound to an HLA complex comprising a β2M polypeptide and an A*1101 HLA-A heavy chain. Such peptides may also be presented in complex with an HLA complex comprising a β2M polypeptide and an A*6801 HLA-A heavy chain.

As non-limiting examples, KRAS peptides comprising a peptide selected from the group consisting of LVVVGADGV (SEQ ID NO:124), LVVVGAVGV (SEQ ID NO:125), LVVVGACGV (SEQ ID NO:126), KLVVVGADGV (SEQ ID NO:127), KLVVVGAVGV (SEQ ID NO:128), KLVVVGACGV (SEQ ID NO:129), LLVVGADGV (SEQ ID NO:130), LLVVGAVGV (SEQ ID NO:131), LLVVGACGV (SEQ ID NO:132), FLVVVGADGV (SEQ ID NO:133), FLVVVGAVGV (SEQ ID NO:134), and FLVVVGACGV (SEQ ID NO:135) where the KRAS peptide has a length of 9 amino acids or 10 amino acids, or a length of at least 9 amino acids or 10 amino acids, present an epitope when bound to an HLA complex comprising a β2M polypeptide and an A*0201 HLA-A heavy chain.

As additional examples, the following KRAS peptides can present an epitope when bound to an HLA complex comprising a β2M polypeptide and an HLA-A heavy chain as follows: GAGDVGKSAL (SEQ ID NO:138), which can present an epitope when bound to an HLA complex comprising a β2M polypeptide and a B*3801 HLA-A heavy chain: AGDVGKSAL (SEQ ID NO:139), which can present an epitope when bound to an HLA complex comprising a β2M polypeptide and a B0702, a B*3801, or a B*3901 HLA-A heavy chain: DVGKSALTI (SEQ ID NO:140), which can present an epitope when bound to an HLA complex comprising a β2M polypeptide and a B*5101 HLA-A heavy chain; GAVGVGKSAL (SEQ ID NO:141), which can present an epitope when bound to an HLA complex comprising a β2M polypeptide and a B*0702 or a B*3801 HLA-A heavy chain; AVGVGKSAL (SEQ ID NO:142), which can present an epitope when bound to an HLA complex comprising a β2M polypeptide and a B*0702 HLA-A heavy chain: YKLVVVGAV (SEQ ID NO:143), which can present an epitope when bound to an HLA complex comprising a β2M polypeptide and an A*0203 or a B*3902 HLA-A heavy chain: ARGVGKSAL (SEQ ID NO:144), which can present an epitope when bound to an HLA complex comprising a β2M polypeptide and a B*0702, a B*2705, or a B*3901 HLA-A heavy chain; GARGVGKSAL (SEQ ID NO:145), which can present an epitope when bound to an HLA complex comprising a β2M polypeptide and a B*0702 HLA-A heavy chain; EYKLVVVGAR (SEQ ID NO:146), which can present an epitope when bound to an HLA complex comprising a β2M polypeptide and an A*3101 HLA-A heavy chain; RGVGKSALTI (SEQ ID NO:147), which can present an epitope when bound to an HLA complex comprising a β2M polypeptide and a B*0702 HLA-A heavy chain; LVVVGARGV (SEQ ID NO:148), which can present an epitope when bound to an HLA complex comprising a β2M polypeptide and an A*0203 HLA-A heavy chain; GADGVGKSAL (SEQ ID NO:149), which can present an epitope when bound to an HLA complex comprising a β2M polypeptide and a B*3801 HLA-A heavy chain; ACGVGKSAL (SEQ ID NO:150), which can present an epitope when bound to an HLA complex comprising a β2M polypeptide and a B*0702 HLA-A heavy chain; and GACGVGKSAL (SEQ ID NO:151), which can present an epitope when bound to an HLA complex comprising a β2M polypeptide and a B*3801 HLA-A heavy chain.

Whether a given peptide (e.g., a KRAS peptide that comprises a KRAS epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays. ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### MHC Polypeptides

As noted above, a TMP includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC class I heavy chain).

In some cases, the first MHC polypeptide is an MHC class I beta-2 microglobulin (β2M) polypeptide, and the second MHC polypeptide is an MHC class I heavy chain (H chain) (“MHC-H”)). In other instances, the first MHC polypeptide is an MHC class I heavy chain polypeptide; and the second MHC polypeptide is a β2M polypeptide. In some cases, both the β2M and MHC-H chain are of human origin; i.e., the MHC-H chain is an HLA heavy chain, or a variant thereof. Unless expressly stated otherwise, a TMP does not include membrane anchoring domains (transmembrane regions) of an MHC class I heavy chain, or a part of MHC class I heavy chain sufficient to anchor the resulting TMP to a cell (e.g., eukaryotic cell such as a mammalian cell) in which it is expressed. In some cases, the MHC class I heavy chain present in a TMP does not include a signal peptide, a transmembrane domain, or an intracellular domain (cytoplasmic tail) associated with a native MHC class I heavy chain. Thus, e.g., in some cases, the MHC class I heavy chain present in a TMP includes only the α1, α2, and α3 domains of an MHC class I heavy chain. In some cases, the MHC class I heavy chain present in a TMP has a length of from about 270 amino acids (aa) to about 290 aa. In some cases, the MHC class I heavy chain present in a TMP has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

### MHC Class I Heavy Chains

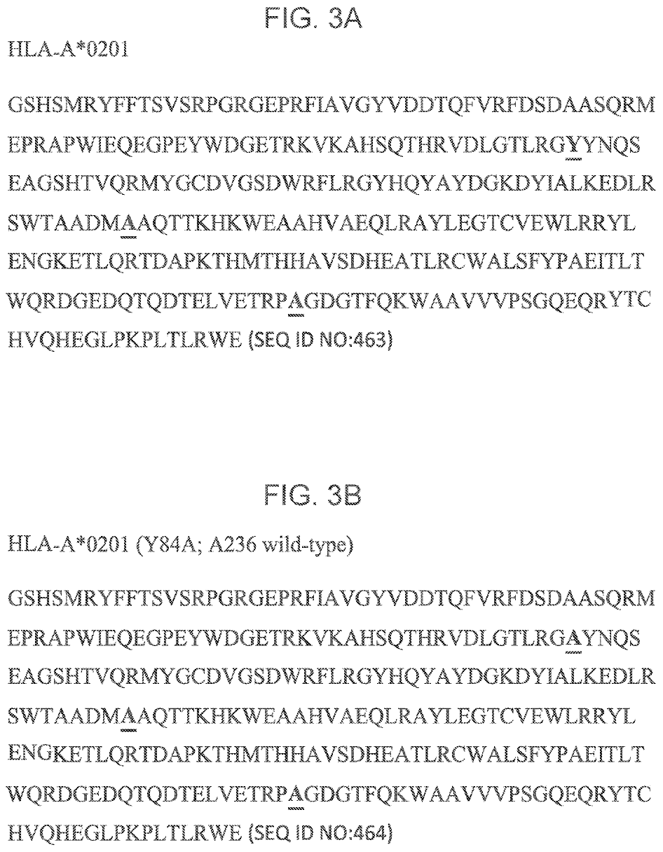

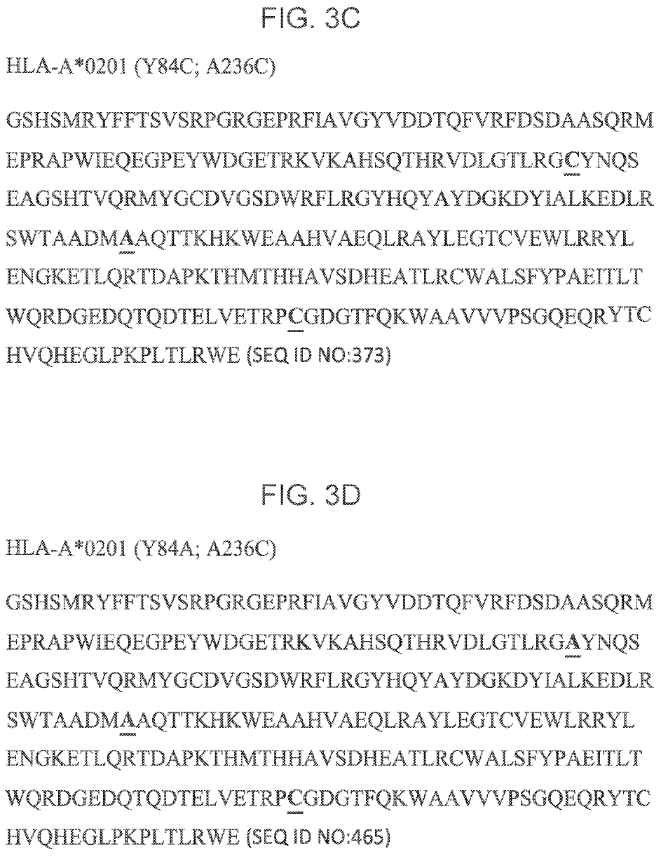

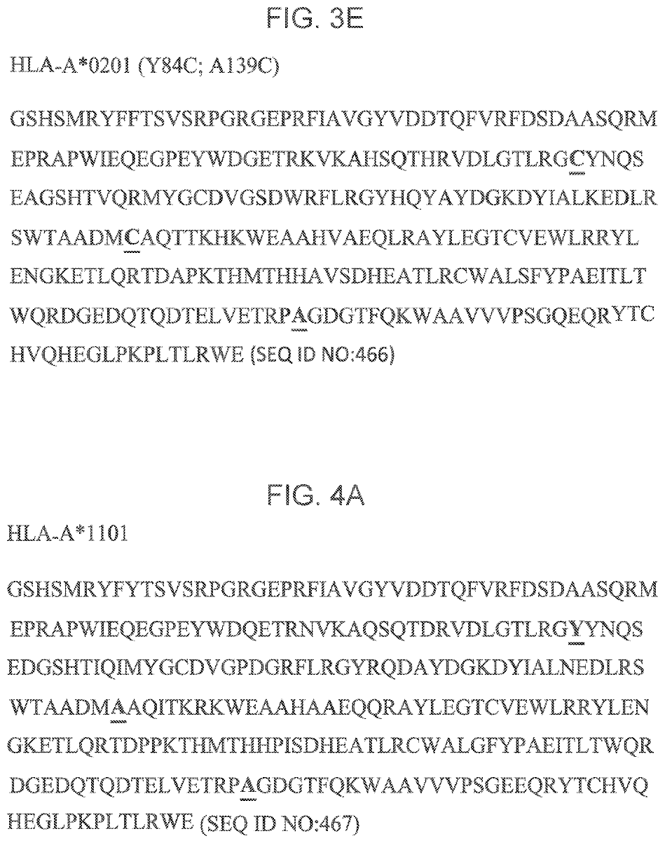

In some cases, an MHC class I heavy chain polypeptide present in a TMP comprises an amino acid sequence having at least 75%, at least 80%, at least 85%, at least 90%, at least 95%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 3-11. In some cases, the MHC class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa. In some cases, an MHC class I heavy chain polypeptide present in a TMP comprises 1-30, 1-5, 5-10, 10-15, 15-20, 20-25 or 25-30 amino acid insertions, deletions, and/or substitutions (in addition to those locations indicated as being variable in the heavy chain consensus sequences) of any one of the amino acid sequences depicted in FIG. 3-11. As noted above, the MHC class I heavy chain typically does not include transmembrane or cytoplasmic domains. As an example, an MHC class I heavy chain polypeptide of a TMP can comprise an amino acid sequence having at least 75%, at least 80%, at least 85%, at least 90%, at least 95%, at least 98%, at least 99%, or 100%, amino acid sequence identity to amino acids 25-300 (lacking all, or substantially all, of the leader, transmembrane and cytoplasmic sequence) or amino acids 25-365 (lacking the leader) of a human HLA-A heavy chain polypeptide.

In some cases, an MHC polypeptide of a TMP is a human MHC polypeptide, where human MHC polypeptides are also referred to as “human leukocyte antigen” (“HLA”) polypeptides. In some cases, an MHC polypeptide of a TMP is a Class I HLA polypeptide. e.g., a β2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

In some cases, a TMP comprises an HLA-A heavy chain polypeptide. The HLA-A heavy chain peptide sequences, or portions thereof, that may be that may be incorporated into a TMP include, but are not limited to, the alleles: A*0101, A*0201, A*0301, A*1101, A*2301, A*2402, A*2407, A*3303, and A*3401. Any of those alleles may comprise a mutation at one or more of positions 84, 139, and 236 (as shown in FIG. 3-6 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, HLA-A sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-A alleles may also be employed (e.g., it may comprise 1-25, 1-5, 5-10, 10-15, 15-20, 20-25, or 25-30 amino acid insertions, deletions, and/or substitutions). Some examples are provided below.

### HLA-A02

In some cases, an MIC class I heavy chain polypeptide of a TMP can comprise an amino acid sequence having at least 75%, at least 80%, at least 85%, at least 90%, at least 95%, at least 98%, at least 99%, or 100%, amino acid sequence identity to the amino acid sequence depicted in FIG. 3A. In some cases, an HLA-A heavy chain polypeptide suitable for inclusion in a TMP comprises the amino acid sequence depicted in FIG. 3A. This HLA-A heavy chain polypeptide is also referred to as “HLA-A*0201” or simply “HLA-A02.” As described in WO 2018/119114 and WO 2020/132138. TMPs can comprise one or more mutations from the wild-type HLA-A02, including to provide Cys residues that can form disulfide bonds. e.g., (i) between the β2M and MHC class I heavy chain, and/or (ii) between the MHC heavy chain and a linker that joins the peptide epitope to the β2M polypeptide, and/or (iii) an intrachain disulfide bond within the MHC class I heavy chain polypeptide.

To facilitate the formation of such disulfide bonds, one or more non-naturally occurring Cys residues can be provided in the heavy chain polypeptide. For example, the MHC class I heavy chain polypeptide comprises an amino acid sequence having at least 75%, at least 80%, at least 85%, at least 90%, at least 95%, at least 98%, at least 99%, or 100%, amino acid sequence identity to
A) the HLA-A02 (Y84C; A236C) amino acid sequence depicted in FIG. 3C, where amino acid 84 is a Cys. and where amino acid 236 is a Cys. In some cases. Cys-84 forms an intrachain disulfide bond with Cys-236; or
B) the HLA-A02 (Y84A; A236C) amino acid sequence: depicted in FIG. 3D, where amino acid 84 is Ala and amino acid 236 is Cys. In some cases, the Cys-236 forms an interchain disulfide bond with Cys-12 of a variant β2M polypeptide that comprises an R12C substitution; or
C) the HLA-A02 (Y84C; A139C) amino acid sequence depicted in FIG. 3E, where amino acid 84 is Cys and amino acid 139 is Cys. In some cases. Cys-84 forms an intrachain disulfide bond with Cys-139.

As noted above, in some cases the MHC class I heavy chain polypeptide comprises an amino acid sequence having at least 75%, at least 80%, at least 85%, at least 90%, at least 95%, at least 98%, at least 99%, or 100%, amino acid sequence identity to the HLA-A02 (Y84A; A236 wild-type) amino acid sequence depicted in FIG. 3B, where amino acid 84 is a Tyr, and amino acid 236 is an Ala.

### HLA-A11 (HLA-A*1101)

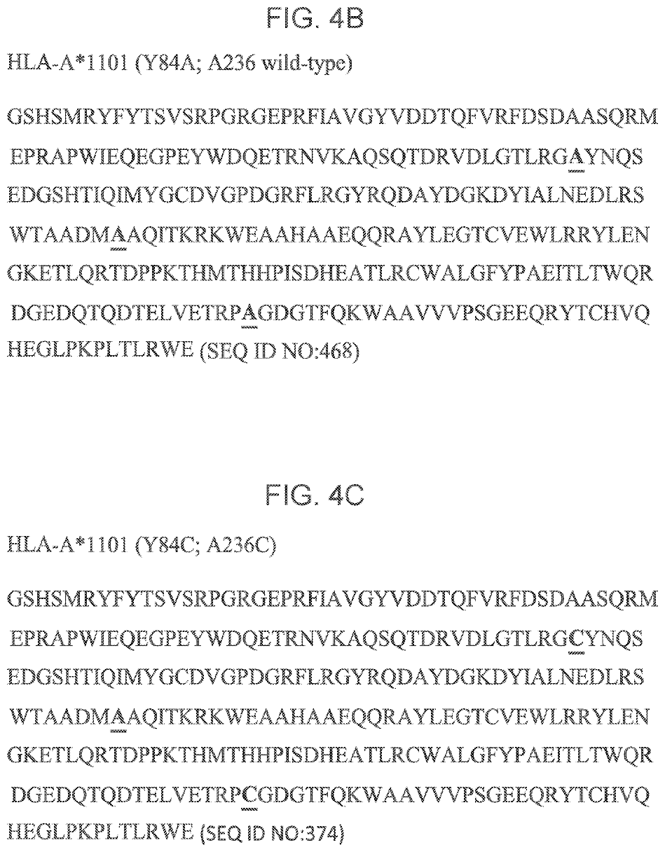

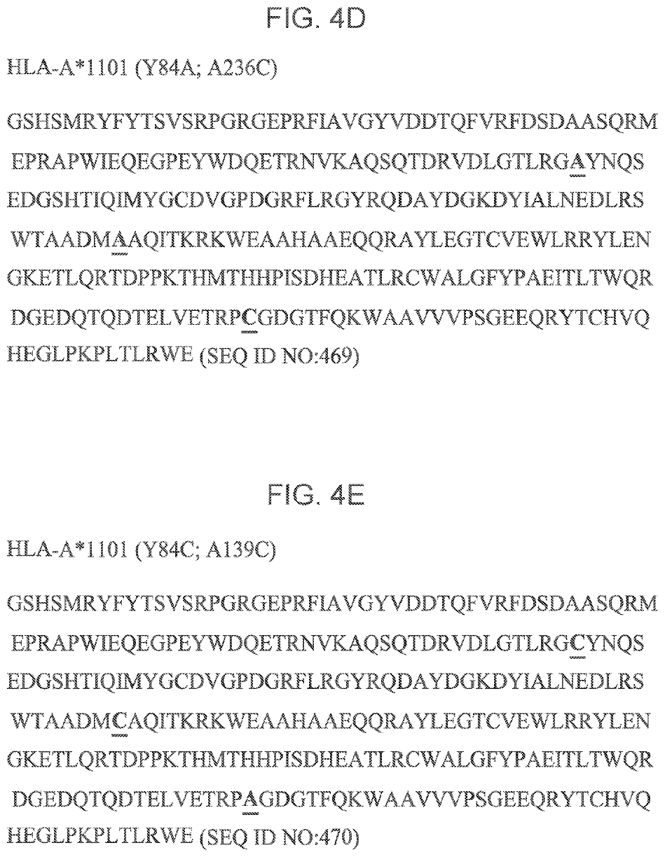

In some cases, an MHC class I heavy chain polypeptide of a TMP can comprise an amino acid sequence having at least 75%, at least 80%, at least 85%, at least 90%, at least 95%, at least 98%, at least 99%, or 100%, amino acid sequence identity to the amino acid sequence depicted in FIG. 4A. In some cases, an HLA-A heavy chain polypeptide suitable for inclusion in a TMP comprises the amino acid sequence depicted in FIG. 4A. This HLA-A heavy chain polypeptide is also referred to as “HLA-A*1101” or simply “HLA-A11” Variants can include, e.g., the MHC class I heavy chain polypeptide comprises an amino acid sequence having at least 75%, at least 80%, at least 85%, at least 90%, at least 95%, at least 98%, at least 99%, or 100%, amino acid sequence identity to:
A) the HLA-A11 (Y84A; A236 wild-type) amino acid sequence depicted in FIG. 4B, where amino acid 84 is a Tyr, and amino acid 236 is an Ala;
B) the HLA-A11 (Y84C; A236C) amino acid sequence depicted in FIG. 4C, where amino acid 84 is a Cys, and where amino acid 236 is a Cys;
C) the HLA-A11 (Y84A; A236C) amino acid sequence: depicted in FIG. 4D, where amino acid 84 is Ala and amino acid 236 is Cys. In some cases, the Cys-236 forms an interchain disulfide bond with Cys-12 of a variant β2M polypeptide that comprises an R12C substitution; and
D) the HLA-A11 (Y84C; A139C) amino acid sequence depicted in FIG. 4E, where amino acid 84 is Cys and amino acid 139 is Cys. In some cases. Cys-84 forms an intrachain disulfide bond with Cys-139.

### HLA-A24(HLA-A*2402)

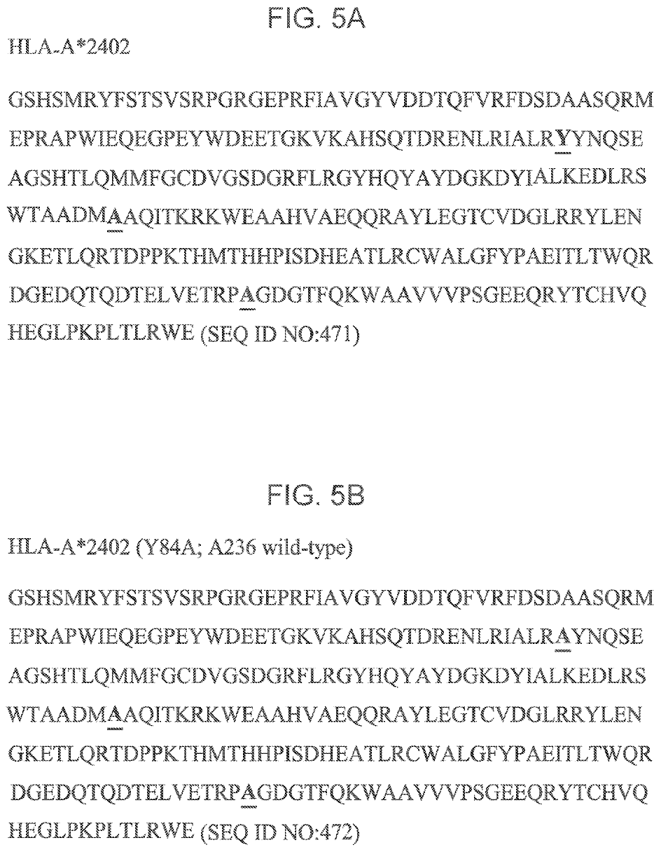

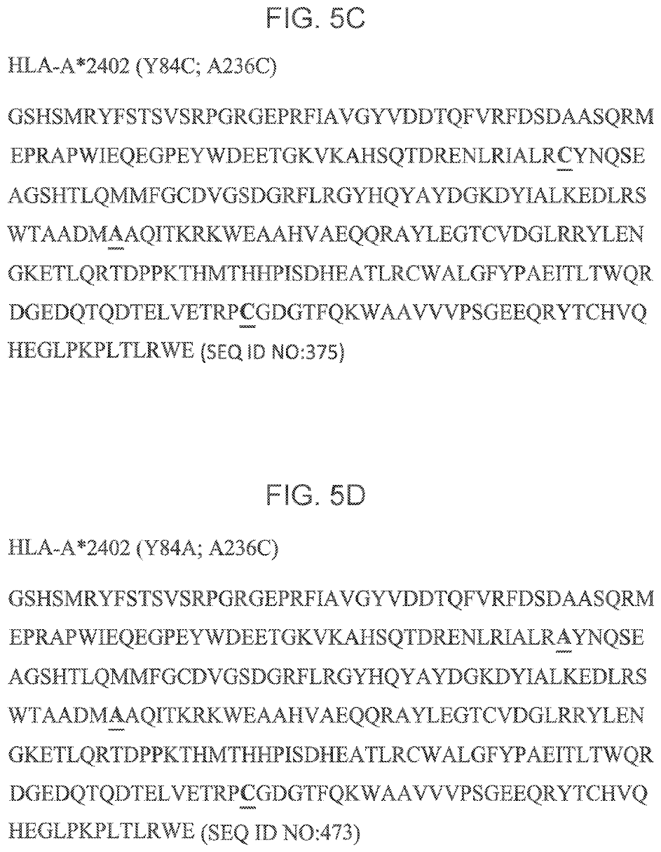

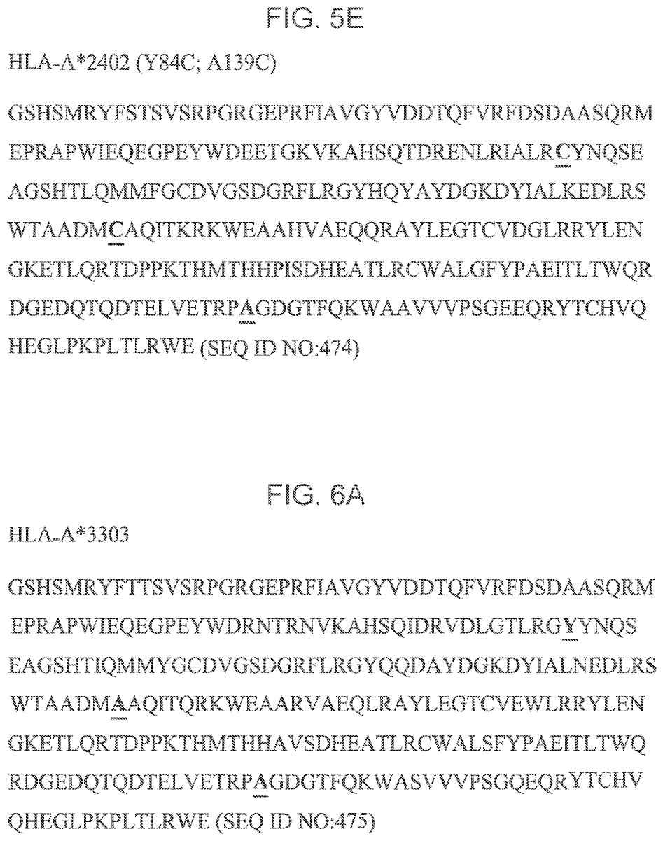

In some cases, an MHC class I heavy chain polypeptide of a TMP can comprise an amino acid sequence having at least 75%, at least 80%, at least 85%, at least 90%, at least 95%, at least 98%, at least 99%, or 100%, amino acid sequence identity to the amino acid sequence depicted in FIG. 5A. In some cases, an HLA-A heavy chain polypeptide suitable for inclusion in a TMP comprises the amino acid sequence depicted in FIG. 5A. This HLA-A heavy chain polypeptide is also referred to as “HLA-A*2402” or simply “HLA-A24.” Variants can include, e.g., the MHC class I heavy chain polypeptide comprises an amino acid sequence having at least 75%, at least 80%, at least 85%, at least 90%, at least 95%, at least 98%, at least 99%, or 100%, amino acid sequence identity to:
A) the HLA-A24 (Y84A; A236 wild-type) amino acid sequence depicted in FIG. 5B, where amino acid 84 is a Tyr, and amino acid 236 is an Ala;
B) the HLA-A24 (Y84C; A236C) amino acid sequence depicted in FIG. 5C, where amino acid 84 is a Cys, and where amino acid 236 is a Cys;
C) the HLA-A24 (Y84A; A236C) amino acid sequence: depicted in FIG. 5D, where amino acid 84 is Ala and amino acid 236 is Cys. In some cases, the Cys-236 forms an interchain disulfide bond with Cys-12 of a variant β2M polypeptide that comprises an R12C substitution; and
D) the HLA-A24 (Y84C; A139C) amino acid sequence depicted in FIG. 5E, where amino acid 84 is Cys and amino acid 139 is Cys. In some cases, Cys-84 forms an intrachain disulfide bond with Cys-139.

### HLA-A33 (HLA-A*3303)

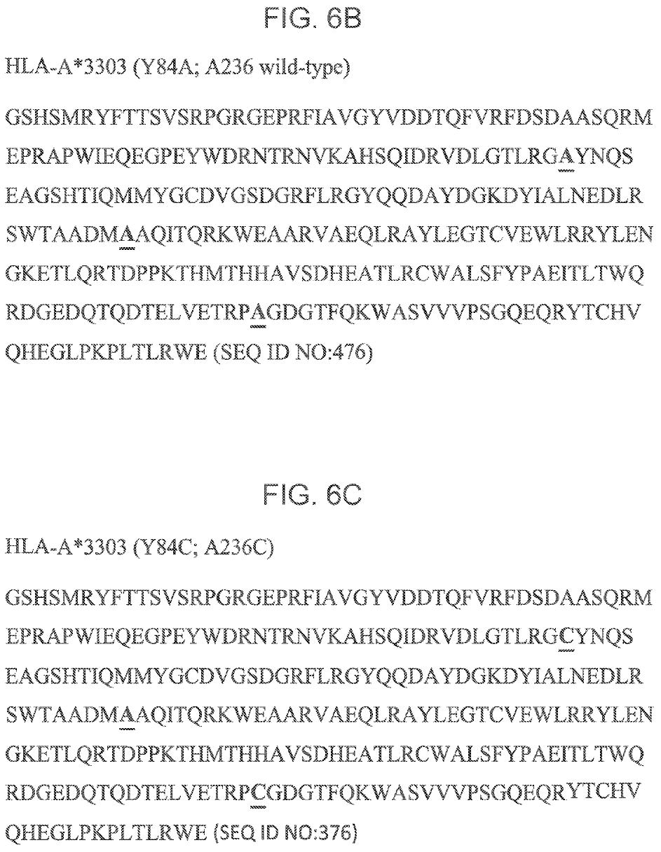

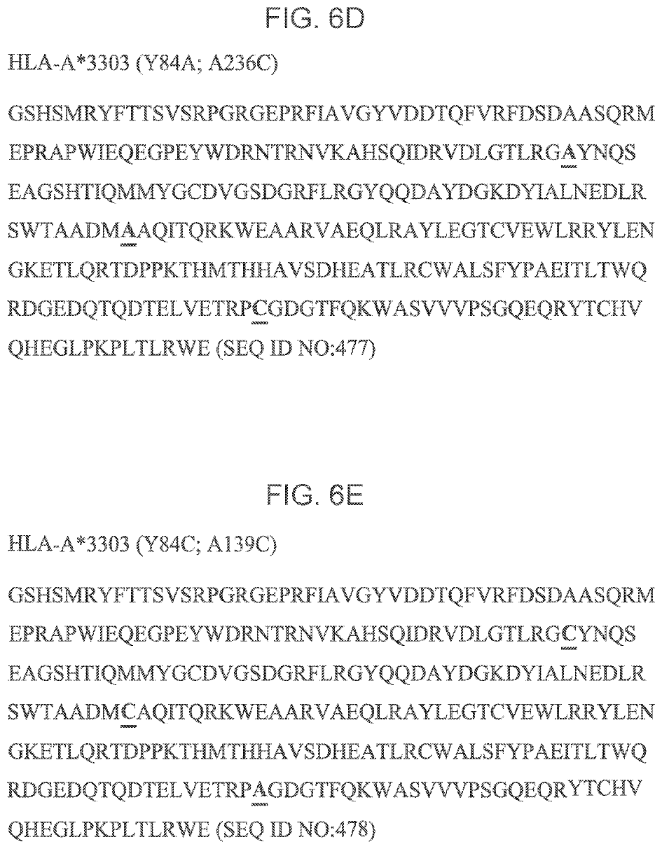

In some cases, an MHC class I heavy chain polypeptide of a TMP can comprise an amino acid sequence having at least 75%, at least 80%, at least 85%, at least 90%, at least 95%, at least 98%, at least 99%, or 100%, amino acid sequence identity to the amino acid sequence depicted in FIG. 6A. In some cases, an HLA-A heavy chain polypeptide suitable for inclusion in a TMP comprises the amino acid sequence depicted in FIG. 6A. This HLA-A heavy chain polypeptide is also referred to as “HLA-A*3303” or simply “HLA-A33.” Variants can include, e.g., the MHC class I heavy chain polypeptide comprises an amino acid sequence having at least 75%, at least 80%, at least 85%, at least 90%, at least 95%, at least 98%, at least 99%, or 100%, amino acid sequence identity to:
A) the HLA-A24 (Y84A; A236 wild-type) amino acid sequence depicted in FIG. 6B, where amino acid 84 is a Tyr, and amino acid 236 is an Ala;
B) the HLA-A24 (Y84C; A236C) amino acid sequence depicted in FIG. 6C, where amino acid 84 is a Cys, and where amino acid 236 is a Cys;
C) the HLA-A24 (Y84A; A236C) amino acid sequence: depicted in FIG. 6D, where amino acid 84 is Ala and amino acid 236 is Cys. In some cases, the Cys-236 forms an interchain disulfide bond with Cys-12 of a variant β2M polypeptide that comprises an R12C substitution; and
D) the HLA-A24 (Y84C; A139C) amino acid sequence depicted in FIG. 6E, where amino acid 84 is Cys and amino acid 139 is Cys. In some cases, Cys-84 forms an intrachain disulfide bond with Cys-139.

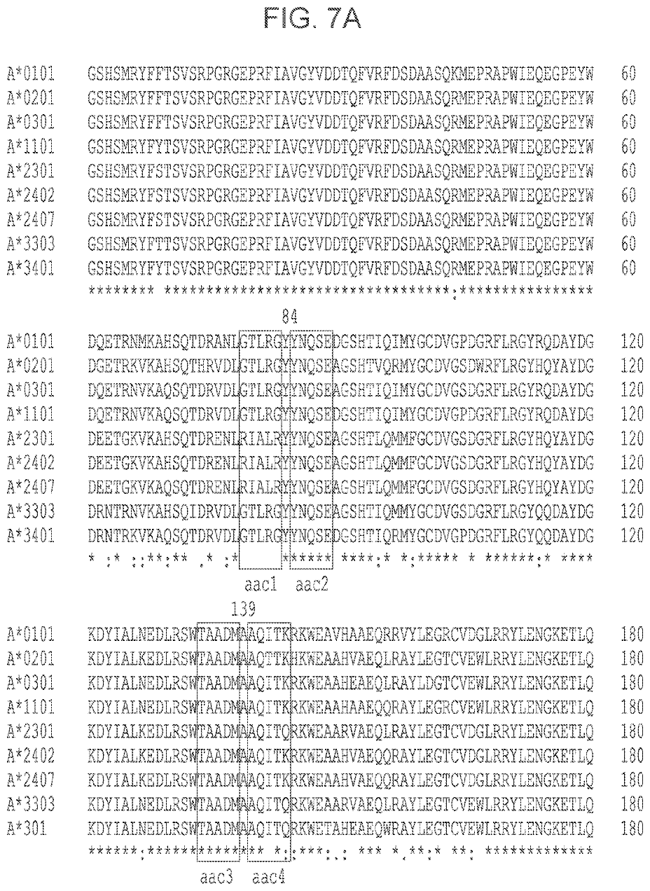

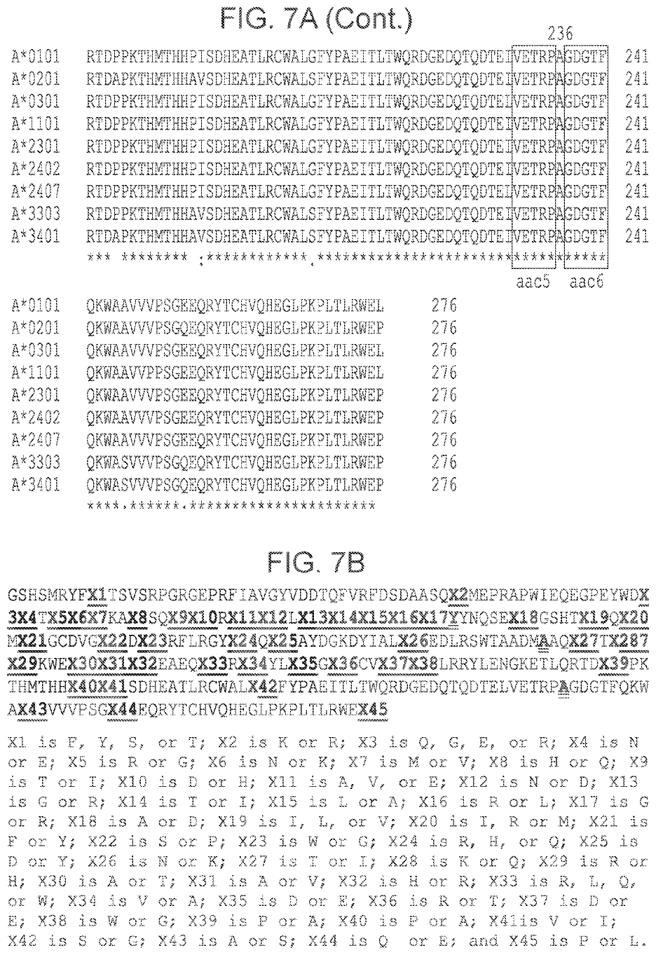

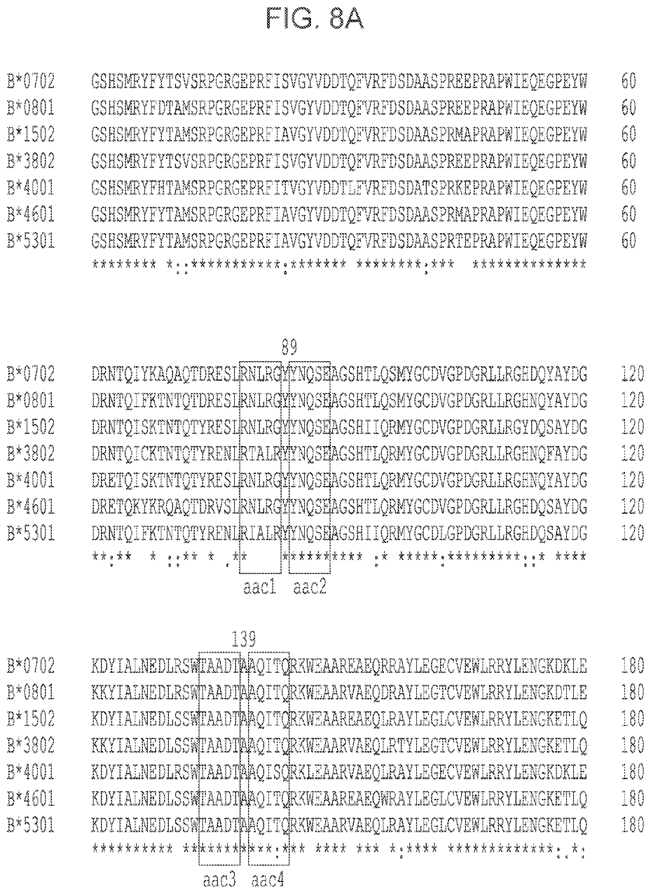

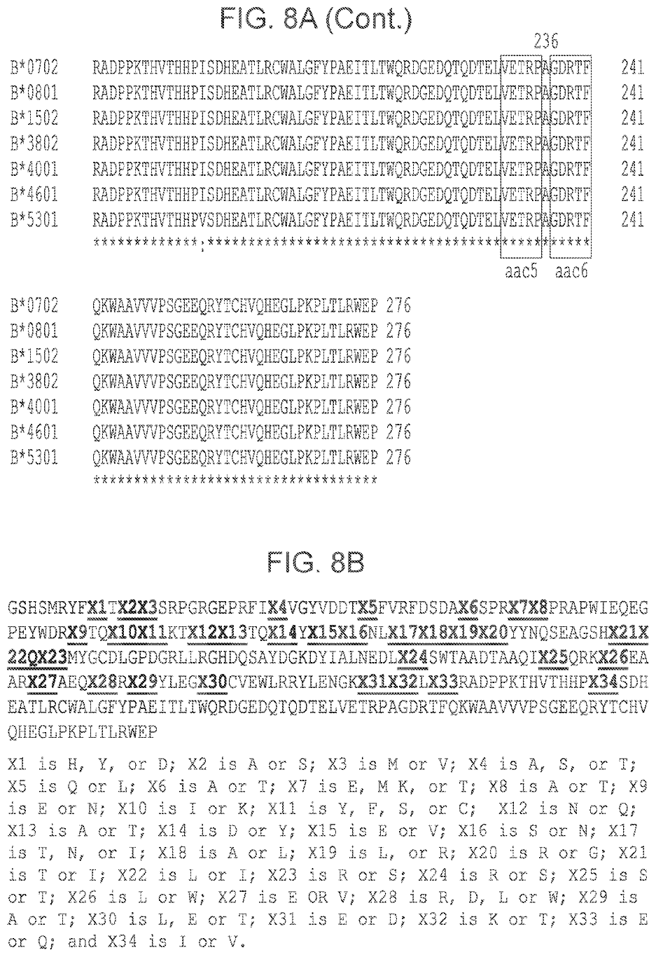

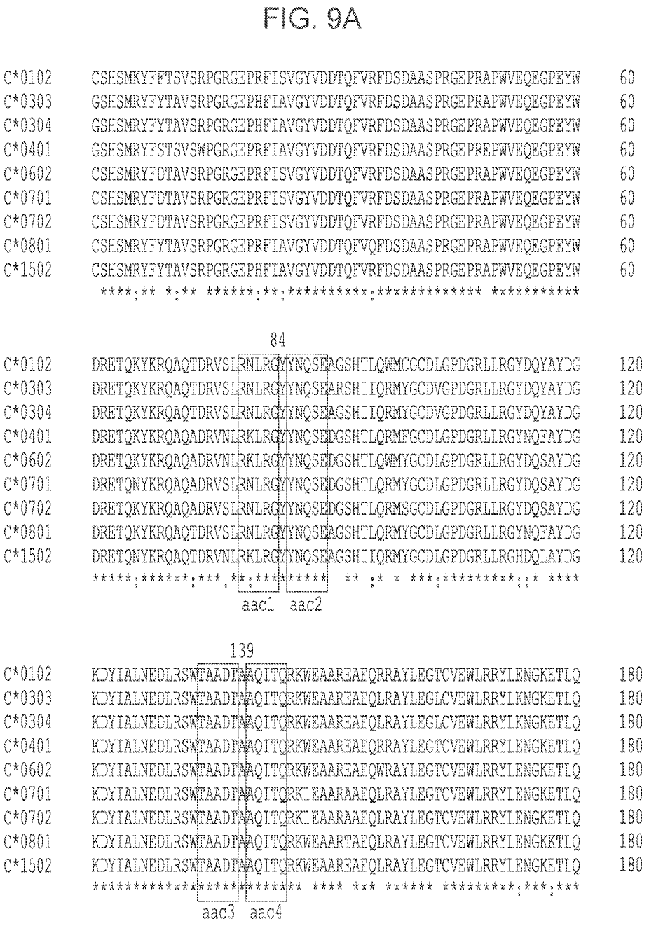

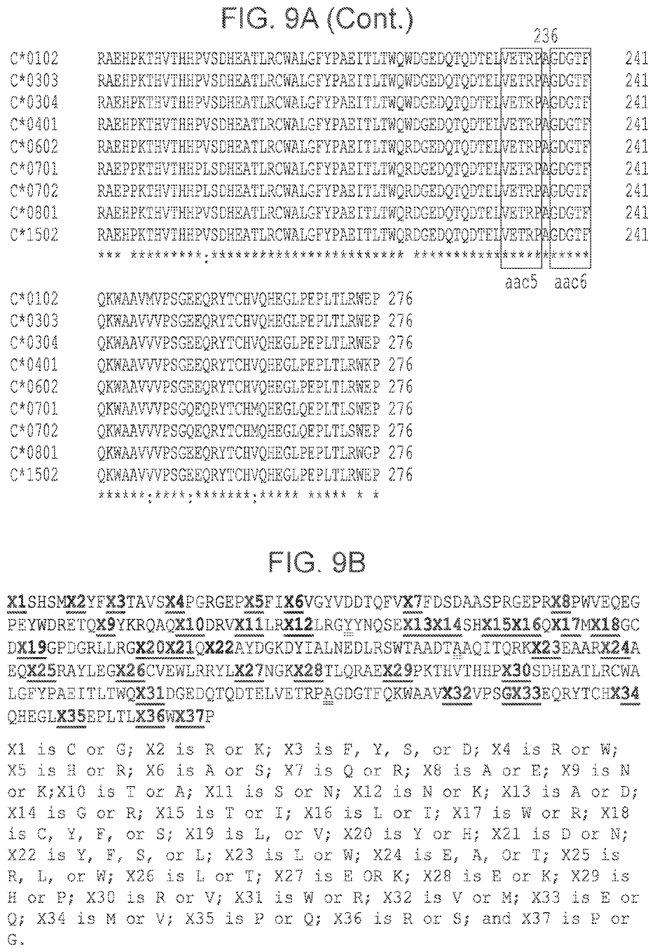

FIGS. 7-9 provide alignments of mature HLA class I heavy chain amino acid sequences (without leader sequences or transmembrane domains or intracellular domains). The aligned amino acid sequences in FIG. 7A are HLA-A class I heavy chains of the following alleles: A*0101, A*0201, A*0301, A*1101, A*2301, A*2402, A*2407, A*3303, and A*3401. The aligned amino acid sequences in FIG. 8A are HLA-B class I heavy chains of the following alleles: B*0702, B*0801, B*1502, B*3802, B*4001, B*4601, and B*5301. The aligned amino acid sequences in FIG. 9A are HLA-C class I heavy chains of the following alleles: C*0102, C*0303, C*0304, C*0401, C*0602, C*0701, C*0801, and C*1502. Indicated in the alignments are the locations (84 and 139 of the mature proteins) where cysteine residues may be introduced (e.g., by substitution) for the formation of a disulfide bond to stabilize the HLA H chain-β2M complex. Also shown in the alignment is position 236 (of the mature polypeptide), which may be substituted by a cysteine residue that can form an inter-chain disulfide bond with β2M (e.g., at aa 12). The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”). that may be replaced by 1 to 5 amino acids selected independently from (i) any naturally occurring amino acid or (ii) any naturally occurring amino acid except proline or glycine.

FIGS. 7A, 8A, and 9A provide alignments of the amino acid sequences of mature HLA-A, -B, and -C class I heavy chains, respectively. The sequences are provided for the extracellular portion of the mature protein (without leader sequences or transmembrane domains or intracellular domains). The positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine ac also shown. FIGS. 7B, 8B, and 9B provide consensus amino acid sequences for the HLA-A, -B, and -C sequences, respectively, provide in FIGS. 7A, 8A, and 9A. The consensus sequences show the variable amino acid positions as “X” residues sequentially numbered and the locations of amino acids 84, 139 and 236 double underlined.

With regard to FIG. 7A, in some cases: i) aac1 (amino acid cluster 1) may be the amino acid sequence GTLRG (SEQ ID NO:158) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:159) or that sequence with one or two amino acids deleted or substituted with other naturally occurring amino acids (e.g., N replaced by Q, Q replaced by N. and/or E replaced by D); iii) aac3 (amino acid cluster 3) may be the amino acid sequence TAADM (SEQ ID NO:160) or that sequence with one or two amino acids deleted or substituted with other naturally occurring amino acids (e.g., T replaced by S. A replaced by G, D replaced by E. and/or M replaced by L, V, or I); iv) aac4 (amino acid cluster 4) may be the amino acid sequence AQTTK (SEQ ID NO:161) or that sequence with one or two amino acids deleted or substituted with other naturally occurring amino acids (e.g., A replaced by G. Q replaced by N. or T replaced by S, and or K replaced by R or Q); v) aac5 (amino acid cluster 5) may be the amino acid sequence VETRP (SEQ ID NO:162) or that sequence with one or two amino acids deleted or substituted with other naturally occurring amino acids (e.g., V replaced by I or L, E replaced by D. T replaced by S, and/or R replaced by K); and/or vi) aac6 (amino acid cluster 6) may be the amino acid sequence GDGTF (SEQ ID NO:163) or that sequence with one or two amino acids deleted or substituted with other naturally occurring amino acids (e.g., D replaced by E, T replaced by S. or F replaced by L, W, or Y).

With regard to FIG. 8A, in some cases: i) aac1 (amino acid cluster 1) may be the amino acid sequence RNLRG (SEQ ID NO:164) or that sequence with one or two amino acids deleted or substituted with other naturally occurring amino acids (e.g., N replaced by T or I; and/or L replaced by A; and/or the second R replaced by L; and/or the G replaced by R); ii) aac2 (amino acid cluster 2) may be the amino acid sequence YNQSE (SEQ ID NO:159) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:165) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:166) or that sequence with one or two amino acids deleted or substituted with other naturally occurring amino acids (e.g., A replaced by G; and/or the first Q replaced by N; and/or I replaced by L or V; and/or the T replaced by S; and/or the second Q replaced by N); v) aac5 (amino acid cluster 5) may be the amino acid sequence VETRP (SEQ ID NO:162) or that sequence with one or two amino acids deleted or substituted with other naturally occurring amino acids (e.g., V replaced by I or L. E replaced by D. T replaced by S, and/or R replaced by K); and/or vi) aac6 (amino acid cluster 6) may be the amino acid sequence GDRTF (SEQ ID NO:167) or that sequence with one or two amino acids deleted or substituted with other naturally occurring amino acids (e.g., D replaced by E; and/or T replaced by S; and/or R replaced by K or H; and/or F replaced by L, W, or Y).

With regard to FIG. 9A, in some cases: i) aac1 (amino acid cluster 1) may be the amino acid sequence RNLRG (SEQ ID NO:164) or that sequence with one or two amino acids deleted or substituted with other naturally occurring amino acids (e.g., N replaced by K; and/or L replaced by A or I; and/or the second R replaced by H; and/or the G replaced by T or S); ii) aac2 (amino acid cluster 2) may be the amino acid sequence YNQSE (SEQ ID NO:159) or that sequence with one or two amino acids deleted or substituted with other naturally occurring amino acids (e.g., N replaced by Q. Q replaced by N, and/or E replaced by D); iii) aac3 (amino acid cluster 3) may be the amino acid sequence TAADT (SEQ ID NO:165) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:166) or that sequence with one or two amino acids deleted or substituted with other naturally occurring amino acids (e.g., A replaced by G; and/or the first Q replaced by N; and/or I replaced by L; and/or the second Q replaced by N or K); v) aac5 (amino acid cluster 5) may be the amino acid sequence VETRP (SEQ ID NO:162) or that sequence with one or two amino acids deleted or substituted with other naturally occurring amino acids (e.g., V replaced by I or L. E replaced by D. T replaced by S, and/or R replaced by K or H); and/or vi) aac6 (amino acid cluster 6) may be the amino acid sequence GDGTF (SEQ ID NO:163) or that sequence with one or two amino acids deleted or substituted with other naturally occurring amino acids (e.g., D replaced by E; and/or T replaced by S; and/or F replaced by L, W. or Y).

Non-Classical HLA-E, -F, and -G MHC Class I Heavy Chains

In some cases, a TMP comprises a non-classical MHC class I heavy chain polypeptide. Among the non-classical HLA heavy chain polypeptides, or portions thereof, that may be that may be incorporated into a TMP include, but are not limited to, those of HLA-E, -F, and -G alleles. Amino acid sequences for HLA-E, -F, and -G heavy chain polypeptides, (and the HLA-A, B and C alleles) may be found on the world wide web hla.alleles.org/nomenclature/index.html, the European Bioinformatics Institute (www(dot)ebi(dot)ac(dot)uk), which is part of the European Molecular Biology Laboratory(EMBL), and at the National Center for Biotechnology Information (www(dot)ncbi(dot)nlm(dot)nih(dot)gov).

Non-limiting examples of suitable HLA-E alleles include, but are not limited to, HLA-E*0101 (HLA-E*01:01:01:01). HLA-E*01:03(HLA-E*01:03:01:01), HLA-E*01:04, HLA-E*01:05. HLA-E*01:06, HLA-E*01:07, HLA-E*01:09, and HLA-E*01:10. Of these. isoforms HLA-E*0101 and HLA-E*01.03 are of particular note since these are highly prevalent alleles, and differ by only 1 amino acid (Arg or Gly at position 107). For example, amino acid sequences of suitable HLA-E heavy chain polypeptides are provided in FIG. 18A-18D, where FIG. 18A provides the amino acid sequence of HLA-E*01:01 (wild-type); FIG. 18B provides the amino acid sequence of HLA-E*01:01 with Y84C and A2346C substitutions; FIG. 18C provides the amino acid sequence of HLA-E*01:03 (wild-type); and FIG. 18D provides the amino acid sequence of HLA-E*01:03 with Y84C and A2346C substitutions.

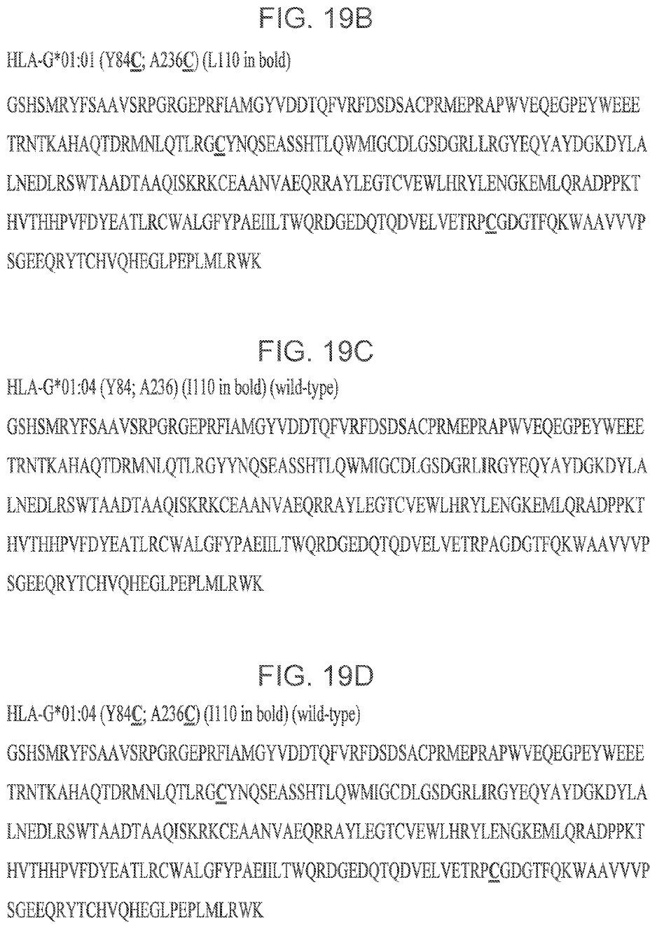

Non-limiting examples of suitable HLA-F alleles include, but are not limited to, HLA-F*0101 (HLA-F*01:01:01.01), HLA-F*01:02. HLA-F*01:03(HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01:03(HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Of these, isoforms HLA-G*0101 (HLA-G*01:01:01:01) and HLA-G*01:04 (HLA-G*01:04:01:01) are of particular note since these are highly prevalent alleles. For example, amino acid sequences of suitable HLA-G heavy chain polypeptides are provided in FIG. 19A-19D, where FIG. 19A provides the amino acid sequence of HLA-G*01:01 (wild-type); FIG. 19B provides the amino acid sequence of HLA-G*01:01 with Y84C and A2346C substitutions; FIG. 19C provides the amino acid sequence of HLA-G*01:04 (wild-type); and FIG. 19D provides the amino acid sequence of HLA-G*01:04 with Y84C and A2346C substitutions.

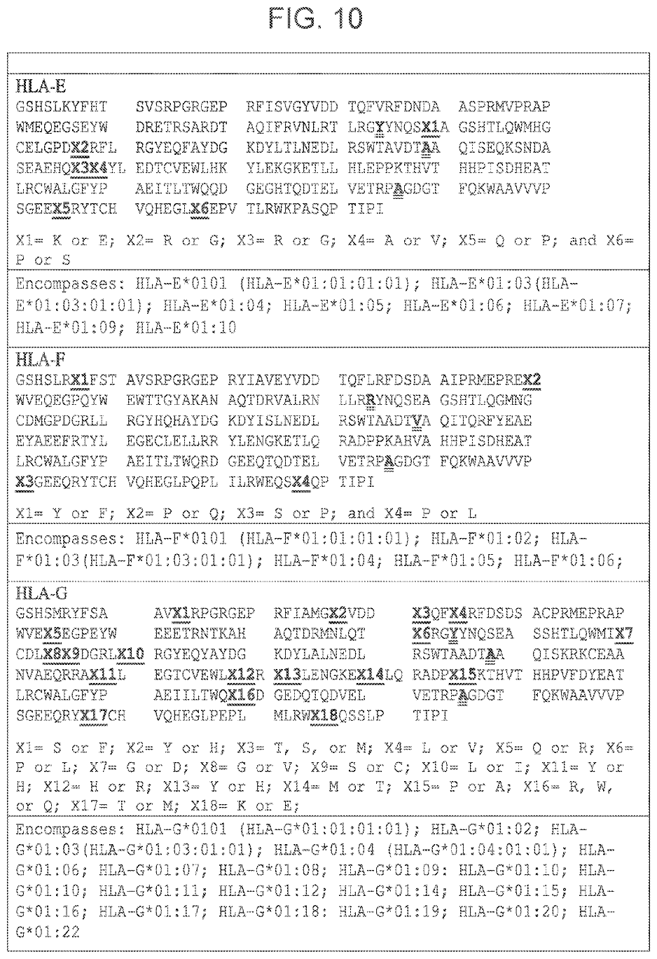

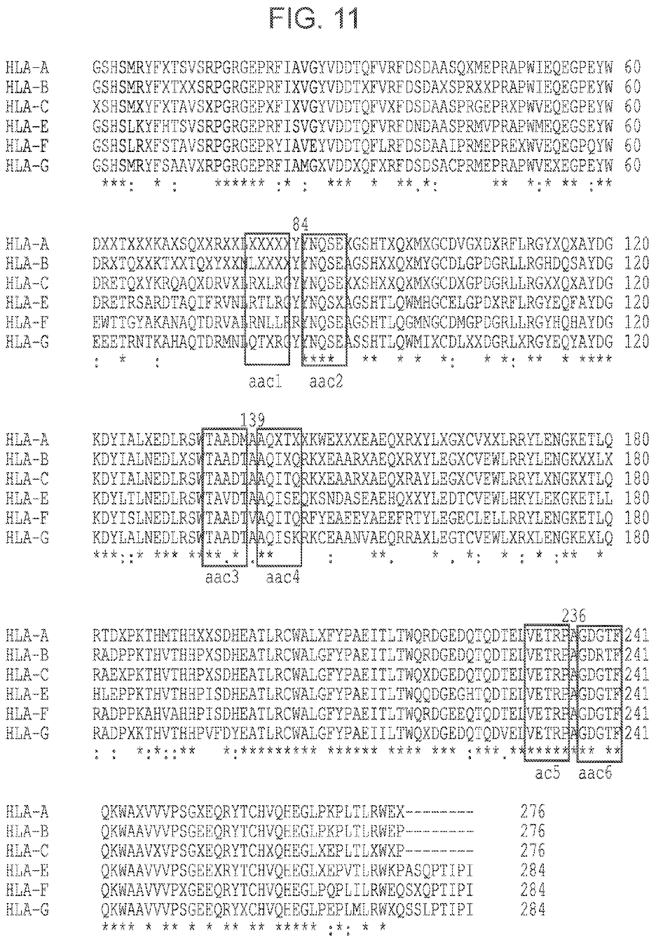

Consensus sequences for those HLA E. —F and -G alleles without all, or substantially all, of the leader. transmembrane and cytoplasmic sequences are provided in FIG. 10, and aligned with consensus sequences of the above-mentioned HLA-A. —B and -C alleles in FIG. 11.

FIG. 10 provides a consensus sequence for each of HLA-E. —F, and -G with the variable aa positions indicated as “X” residues sequentially numbered and the locations of aas 84, 139 and 236 double underlined.

FIG. 11 provides an alignment of the consensus amino acid sequences for HLA-A, -B, -C, -E, -F, and -G, which are given in FIGS. 7-11. Variable residues in each sequence are listed as “X” with the sequential numbering removed. The locations of aas 84, 139 and 236 are indicated with their flanking five-amino acid clusters that may be replaced by 1 to 5 amino acids selected independently from (i) any naturally occurring amino acid or (ii) any naturally occurring amino acid except proline or glycine are also shown.

Any of the above-mentioned HLA-E, -F, and/or -G alleles may comprise a substitution at one or more of positions 84, 139 and/or 236 as shown in FIG. 11 for the consensus sequences. In some cases, the substitutions may be selected from a: position 84 tyrosine to alanine (Y84A) or cysteine (Y84C), or, in the case of HLA-F, an R84A or R84C substitution; a position 139 alanine to cysteine (A139C), or, in the case of HLA-F, a V139C; and an alanine to cysteine substitution at position 236 (A236C). In addition, an HLA-E, -F and/or -G sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of their respective consensus sequences set forth in FIG. 11 may also be employed (e.g., the sequences may comprise 1-25, 1-5, 5-10, 10-15, 15-20, 20-25, or 25-30 amino acid insertions, deletions, and/or substitutions in addition to changes at variable residues listed therein).

Beta-2 Microglobulin

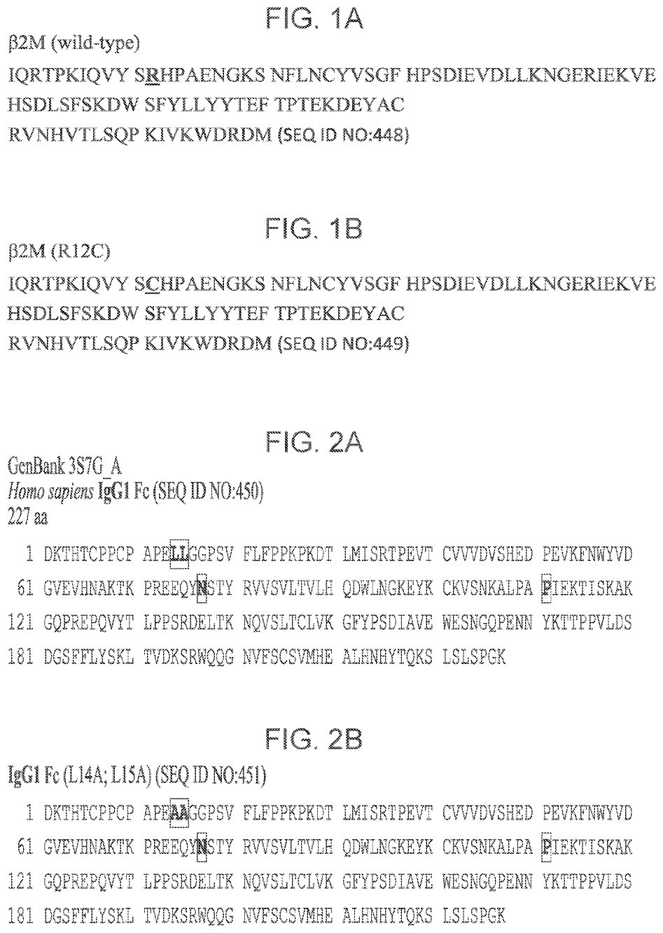

A β2-microglobulin (β2M) polypeptide of a TMP can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to the amino acid sequence depicted in FIG. 1A (wild-type human β2M). In some cases, a β2M polypeptide present in a TMP comprises the amino acid sequence: depicted in FIG. 1A (wild-type human β2M).

In some cases, an MHC polypeptide present in a TMP comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such cysteine residues can form a disulfide bond with a naturally occurring or non-naturally occurring cysteine residue present in the MHC heavy chain of the TMP. As used herein, a reference to a “non-naturally occurring Cys residue” in an MHC class I polypeptide means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

In some cases, a β2M polypeptide present in a TMP comprises an amino acid sequence having at least 75%, at least 80%, at least 85%, at least 90%, at least 95%, at least 98%, at least 99%, or 100%, amino acid sequence identity to the amino acid sequence depicted in FIG. 1B, where amino acid 12 is a Cys; i.e., where the β2M comprises a non-naturally-occurring Cys at position 12 as a result of an R12C substitution. In some cases, a β2M polypeptide present in a TMP comprises the amino acid sequence depicted in FIG. 1B.

### Intrachain and Interchain Disulfide Bonds

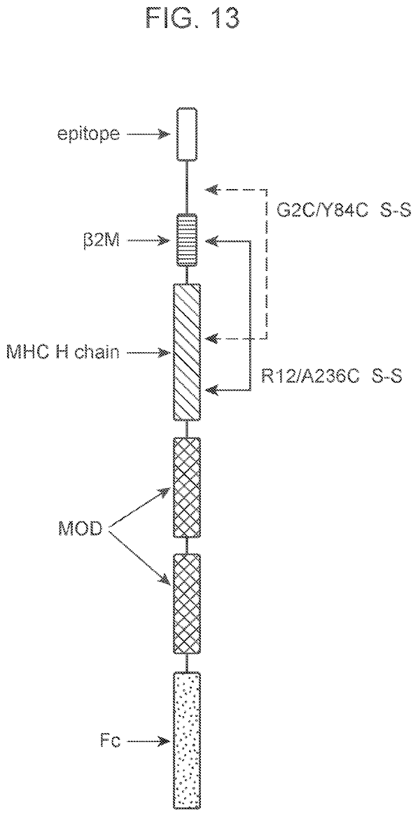

In some cases, a TMP comprises one or more intrachain disulfide bonds. FIG. 13 provides a schematic depiction of such a disulfide-linked TMP. In addition, where TMP comprises an Ig Fc polypeptide, a TMP can be dimerized, such that one or more interchain disulfide bonds link the Ig Fc polypeptides in the two TMPs, to form a homodimer. In some cases, a dimerized TMP comprises: i) interchain disulfide bonds (e.g., one or two disulfide bonds between Ig Fc polypeptides present in two TMPs); and ii) one or more intrachain disulfide bonds.

In some cases where a KRAS peptide of a TMP is linked to a β2M polypeptide by a linker comprising a Cys, at least one of the one or more intrachain disulfide bonds links the Cys in the linker to a Cys in an MHC class I heavy chain in TMP. In some cases, a TMP comprises a Cys in a β2M polypeptide and a Cys in MHC class I heavy chain; and the TMP comprises an intrachain disulfide bond linking the Cys in the β2M polypeptide to the Cys in the MHC class I heavy chain. In some cases, a TMP comprise: a) a first intrachain disulfide bond linking: i) a Cys present in a linker between a KRAS peptide and a β2M polypeptide in the TMP; and ii) a first Cys present in an MHC class I heavy chain in the TMP; and b) a second intrachain disulfide bond linking: i) a Cys present in a β2M polypeptide in the TMP; and it) a second Cys in the MHC class I heavy chain. Cys-containing linkers am discussed in more detail below.

Generally speaking, potential locations in a TMP for disulfide bonds are where residues in the TMP are separated by a distance of 5 angstroms or less. Such locations represent potential locations where Cys residues, if not naturally present, can be substituted for the residues that exist in the polypeptides. For example. Cys residues in a TMP potentially can be linked via a disulfide bond between two Cys residues that are generally no more than about 5 angstroms apart from one another in the TMP. In some cases, one or both of the Cys residues are non-naturally occurring. An amino acid in the β2M polypeptide and MHC class I heavy chain of TMPs that are no more than 5 angstroms from one another represent amino acids that, when substituted with a Cys, can form a disulfide bond in a TMP. Similarly, a disulfide bond can be formed between a Cys residue in a linker and a naturally occurring or non-naturally occurring Cys residue in an MHC heavy chain where the two Cys residues are no more than about 5 angstroms apart from each other. Notably, however, not all pairs of residues separated by about 5 angstroms or less will be suitable for formation of a disulfide bond or provide a disulfide bond that stabilizes the resulting TMP or provides enhanced expression.

A TMP can comprise, for example: i) a KRAS peptide (e.g., a KRAS peptide of from 4 amino acids to about 25 amino acids in length, that is bound by a TCR when the peptide is complexed with MHC polypeptides); ii) a first MHC polypeptide; iii) a peptide linker between the KRAS peptide and the first MHC polypeptide, where the peptide linker comprises a Cys residue, and where the first MHC polypeptide is a β2M polypeptide that comprises an amino acid substitution that introduces a Cys residue; iv) a second MHC polypeptide, where the second MHC polypeptide is a Class I heavy chain comprising a Y84C substitution and an A236C substitution, (based on the amino acid numbering of HLA-A*0201 (depicted in FIG. 3A), or at corresponding positions in another Class I heavy chain allele), where the TMP comprises a first intrachain disulfide bond between the Cys residue in the peptide linker (the peptide linker between the KRAS peptide and the first MHC polypeptide) and the Cys residue at amino acid position 84 of the MHC class I heavy chain polypeptide, and where the TMP comprises a second intrachain disulfide bond between the introduced Cys residue in the β2M polypeptide and the Cys at amino acid position 236 of the MHC class I heavy chain polypeptide; v) at least one MOD; and, optionally, vi) an Ig Fc polypeptide. Such intrachain disulfide bonds are depicted schematically in FIG. 13.

Non-limiting examples of MHC class I heavy chain comprising a Y84C substitution and an A236C substitution. based on the amino acid numbering of HLA-A*0201 (depicted in FIG. 3A), or at corresponding positions in another Class I heavy chain allele, are depicted in FIG. 4C, FIG. 5C, and FIG. 6C.

In some cases, a TMP comprises an HLA-A Class I heavy chain polypeptide. In some cases, the HLA-A heavy chain polypeptide present in a TMP (e.g., a TMP comprising one or more intrachain disulfide bonds) comprises an amino acid sequence having at least 95%, at least 98%, or at least 99%, amino acid sequence identity to the HLA-A*0101. HLA-A*0201, HLA-A*0301, HLA-A*1101, HLA-A*2301, HLA-A*2402, HLA-A*2407, HLA-A*3303, or HLA-A*3401 amino acid sequence depicted in FIG. 7A, where the HLA-A heavy chain polypeptide comprises: i) a Y84C substitution; and where amino acid 236 is an Ala (e.g., as depicted in FIG. 3B, FIG. 4B, FIG. 5B, and FIG. 6B); ii) Y84C and A236C substitutions (e.g., as depicted in FIG. 3C, FIG. 4C, FIG. 5C, and FIG. 6C); or iii) a Y84A substitution and an A236C substitution (e.g., as depicted in FIG. 3D, FIG. 4D, FIG. 5D, and FIG. 6D).

In some cases, the HLA-A heavy chain polypeptide present in a TMP comprises an amino acid sequence having at least 95%, at least 98%, at least 99%, or 100%, amino acid sequence identity to one of the following sequences:
(i) the HLA-A*0201 (Y84C; A236C) amino acid sequence shown in in FIG. 3C (SEQ ID NO:20), where amino acid 84 is a Cys and amino acid 236 is a Cys;
(ii) the HLA-A*1101 (Y84C; A236C) amino acid sequence shown in FIG. 4C (SEQ ID NO:25), where amino acid 84 is a Cys and amino acid 236 is a Cys;
(iii) the HLA-A*2402 (Y84C; A236C) amino acid sequence shown in FIG. 5C (SEQ ID NO:30), where amino acid 84 is a Cys and amino acid 236 is a Cys;
(iv) the HLA-A*3303 (Y84C; A236C) amino acid sequence shown in FIG. 6C (SEQ ID NO:35), where amino acid 84 is a Cys and amino acid 236 is a Cys.

In some cases, the HLA-A heavy chain polypeptide present in a TMP comprises an amino acid sequence having at least 95%, at least 98%, or at least 99%, amino acid sequence identity to one of the following sequences:
(i) the HLA-A*0101 amino acid sequence shown in in FIG. 7A (SEQ ID NO:38), where amino acid 84 is a Cys and amino acid 236 is a Cys;
(ii) the HLA-A*0301 amino acid sequence shown in FIG. 7A (SEQ ID NO:40), where amino acid 84 is a Cys and amino acid 236 is a Cys;
(iii) the HLA-A*2301 amino acid sequence shown in FIG. 7A (SEQ ID NO:42), where amino acid 84 is a Cys and amino acid 236 is a Cys;
(iv) the HLA-A*2407 amino acid sequence shown in FIG. 7A (SEQ ID NO:44), where amino acid 84 is a Cys and amino acid 236 is a Cys;
(v) the HLA-A*3401 amino acid sequence shown in FIG. 7A (SEQ ID NO:46), where amino acid 84 is a Cys and amino acid 236 is a Cys.

In some cases, a TMP comprises an HLA-E Class I heavy chain polypeptide. In some cases, the HLA-E heavy chain polypeptide present in a TMP (e.g., a TMP comprising one or more intrachain disulfide bonds) comprises an amino acid sequence having at least 95%, at least 98%, or at least 99%, or 100%, amino acid sequence identity to any one of the amino acid sequences depicted in FIG. 18A-18D.

In some cases, the HLA-E heavy chain polypeptide present in a TMP comprises an amino acid sequence having at least 95%, at least 98%, at least 99%, or 100%, amino acid sequence identity to one of the following sequences:
(i) the HLA-E*01:01 (Y84C; A236C) amino acid sequence shown in in FIG. 18B (SEQ ID NO:100), where amino acid 84 is a Cys and amino acid 236 is a Cys;
(ii) the HLA-E*01:03 (Y84C; A236C) amino acid sequence shown in FIG. 18D (SEQ ID NO:102), where amino acid 84 is a Cys and amino acid 236 is a Cys;

In some cases, a TMP comprises an HLA-G Class I heavy chain polypeptide. In some cases, the HLA-G heavy chain polypeptide present in a TMP (e.g., a TMP comprising one or more intrachain disulfide bonds) comprises an amino acid sequence having at least 95%, at least 98%, or at least 99%, or 100%, amino acid sequence identity to any one of the amino acid sequences depicted in FIG. 19A-19D

In some cases, the HLA-G heavy chain polypeptide present in a TMP comprises an amino acid sequence having at least 95%, at least 98%, at least 99%, or 100%, amino acid sequence identity to one of the following sequences:
(i) the HLA-G*01:01 (Y84C; A236C) amino acid sequence shown in in FIG. 19B (SEQ ID NO:104), where amino acid 84 is a Cys and amino acid 236 is a Cys;
(ii) the HLA-G*01:04 (Y84C; A236C) amino acid sequence shown in FIG. 19D (SEQ ID NO:106), where amino acid 84 is a Cys and amino acid 236 is a Cys;

### HLA/Peptide Binding Assays

Whether a given peptide (e.g., a KRAS peptide that comprises a KRAS epitope) binds a class I HLA (comprising an HLA heavy chain and a β2M polypeptide), and, when bound to the HLA complex. can effectively present an epitope to a TCR, can be determined using any of a number of well-known methods. Assays include binding assays and T-cell activation assays, including cell-based binding assays, biochemical binding assays. T-cell activation assays, ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Immunomodulatory Polypeptides

In some cases, a MOD present in a TMP is a wild-type (“wt.”) MOD. As discussed above, in other cases, a MOD present in a TMP is a variant of a wt. MOD that has reduced affinity for a co-MOD compared to the affinity of a corresponding wild-type MOD for the co-MOD. Suitable MODs that exhibit reduced affinity for a co-MOD can have from 1 amino acid t aa) to 20 aa differences from a wild-type MOD. For example, in some cases, a variant MOD present in a TMP differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMP differs in amino acid sequence by 11 aa, 12 aa, 13 aa, 14 aa, 15 aa, 16 aa, 17 aa, 18 aa, 19 aa, or 20 aa, from a corresponding wild-type MOD.

As discussed above, a MOD may comprise a variant of a wt immunomodulatory polypeptide that may exhibit reduced binding to its co-MOD, including e.g., reduced binding to one or more chains or domains of the co-MOD. For example, a variant MOD present in a TMP may bind its co-MOD with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type MOD for the co-MOD.

Exemplary pairs of MODs and their co-MODs include, but are not limited to those set out in Table 1, below:

TABLE 1

| Immunomodulatory | |
|---|---|
| Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

As depicted schematically in FIG. 12, one or more MODs can be present in a TMP at any of a variety of positions. FIG. 12 depicts the position of two copies of a variant IL-2 polypeptide; however, the MOD can be any number of and any of a variety of MODs, as described herein. As depicted in FIG. 18, a MOD can be: 1) C-terminal to the MHC class I heavy chain and N-terminal to the Ig Fc polypeptide; in other words, between the MHC class I heavy chain polypeptide and the Ig Fc polypeptide, which is referred to as “Position 2” in FIG. 12; 2) C-terminal to the Ig Fc polypeptide, which is referred to as “Position 3” in FIG. 12; or 3) N-terminal to the peptide epitope, which is referred to as “Position 4” in FIG. 12.

Immunomodulatory polypeptides and variants, including reduced affinity variants, such as PD-L1, CD80. CD86, 4-1BBL and IL-2 are described in the published literature, e.g., published PCT application WO2020132138A1 and WO2019/051091, the disclosures of which as they pertain to MODs and specific variant MODs of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

An IL-2 receptor is in some cases a heteromeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2, human IL-2Rα, IL2Rβ, and IL-2Rγ are known. See, e.g., published PCT applications WO2020132138A1 and WO2019/051091, discussed above. For example, a wild-type IL-2 polypeptide can have the amino acid sequence depicted in FIG. 17A. Amino acid sequences of human IL-2Rα, human IL-2Rβ, and human IL-2Rγ are depicted in FIGS. 17B, 17C, and 17D, respectively.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2R and/or IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically hinds the peptide epitope on the TMP. That is, the binding of the IL-2 variant MOD to its costimulatory polypeptide on the T cell is substantially driven by the binding of the MHC-epitope moiety rather than by the binding of the IL-2.

Suitable IL-2 variant MODs thus include a polypeptide that comprises an amino acid sequence having at least 90%, at least 95%, at least 98%, or at least 99% amino acid sequence identity to the wild-type IL-2 amino acid sequence depicted in FIG. 17A; and that have one or more amino acid differences from the wild-type IL-2 amino acid sequence depicted in FIG. 17A. In some cases, such a variant IL-2 polypeptide of this disclosure exhibits reduced binding affinity to IL-2R. compared to the binding affinity of an IL-2 polypeptide comprising the wild-type IL-2 amino acid sequence depicted in FIG. 17A. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least (0% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 17A for an IL-2R (e.g., an IL-2R comprising polypeptides comprising the amino acid sequences depicted in FIG. 17B-17D), when assayed under the same conditions.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 168), i.e., the variant IL-2 polypeptide has the amino acid sequence of wild-type IL-2 but with H16A and F42A substitutions (shown in hold). Alternatively, the foregoing sequence, but with substitutions other than Ala at H16 and/or F42 may be employed, e.g., H16T may be employed instead of H16A. In some cases, a variant IL-2 polypeptide present in a TMP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 168). In some cases, a variant TL-2 polypeptide present in a TMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:169). In some cases, a TMP comprises two copies of such a variant IL-2 polypeptide.

In some cases, a MOD present in a TMP is a PD-L1 polypeptide. In some cases, a PD-L1 polypeptide of a TMP comprises an amino acid sequence having at least 75%, at least 80%, at least 85%, at least 90%, at least 95%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:170).

In some cases, a MOD present in a TMP is a 4-1 BBL polypeptide. In some cases, a 4-1BBL polypeptide of a TMP comprises an amino acid sequence having at least 75%, at least 80%, at least 85%, at least 90%, at least 95%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:171′).

In some cases, a MOD present in a TMP is an ICOS-L polypeptide. In some cases, an ICOS-L polypeptide of a TMP comprises an amino acid sequence having at least 75%, at least 80%, at least 85%, at least 90%, at least 95%, at least 98%, at least 99%, or 100%, amino acid sequence identity to the following ICOS-L amino acid sequence: QEKEVRAMVG SDVELSCACP EGSRFDLNDV YVYWQTSFSK TVVTYHIPQN SSLENVDSRY RNRALMSPAG MLRGDFSLRL FNVTPQDEQK FHCLVLSQSL GFQEVLSVEV rLHVAANFSV PVVSAPHSPS QDELTFTCTS INGYPRPNVY WINKTDNSLL DQALQNDTVF LNMRGLYDVV SVLRIARTPS VNIGCCIENV LLQQNLTVGS QTGNDIGERD KITENPVSTG EKNAATWSIL (SEQ ID NO:172).

In some cases, a T-cell modulatory polypeptide of a multimeric polypeptide is an OX40L polypeptide. In some cases, an OX40L polypeptide of a TMP comprises an amino acid sequence having at least 75%, at least 80%, at least 85%, at least 90%, at least 95%, at least 98%, at least 99%, or 100%, amino acid sequence identity to the following OX40L amino acid sequence: L QVSHRYPRIQ SIKVQFTEYK KEKGFILTSQ KEDEIMKVQN NSVHNCDGF YLISLKGYFS QEVNISLHYQ KDEEPLFQLK KVRSVNSLMV ASLTYKDKVY LNVTTDNTSL DDFHVNGGEL ILIHQNPGEF CVL (SEQ ID NO:173).

In some cases, a T-cell modulatory polypeptide of a multimeric polypeptide is a PD-L2 polypeptide. In some cases, a PD-L2 polypeptide of a multimeric polypeptide comprises an amino acid sequence having at least 75%, at least 80%, at least 85%, at least 90%, at least 95%, at least 98%, at least 99%, or 100%, amino acid sequence identity to amino acids 20-273 of the PD-L2 amino acid sequence:

(SEQ ID NO: 174)
L FTVTVPKELY HIEHGSNVTL ECNEDTGSHV
NLGAITASLQ KVENDTSPHR ERATLLEEQL
PLGKASFHIP QVQVRDEGQY QCIIIYGVAW
DYKYLTLKVK ASYRKINTHI LKVPETDEVE
LTCQATGYPL AEVSWPNVSV PANTSHSRTP
EGLYQVTSVL RLKPPPGRNF SCVFWNTHVR
ELTLASIDLQ SQMEPRTHPT.

In some cases, a MOD present in a TMP is a CD80 polypeptide. In some cases, a CD80 polypeptide of a TMP comprises an amino acid sequence having at least 75%, at least 80%, at least 85%, at least 90%, at least 95%, at least 98%, at least 99%, or 100%, amino acid sequence identity to following CD80 amino acid sequence: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: (SEQ ID NO:175).

In some cases, a MOD present in a TMP is a CD86 polypeptide. In some cases, a CD86 polypeptide of a TMP comprises an amino acid sequence having at least 75%, at least 80%, at least 85%, at least 90%, at least 95%, at least 98%, at least 99%, or 100%, amino acid sequence identity to the following CD86 amino acid sequence:

(SEQ ID NO: 176)
APLKIQAYFNETADLPCQFANSQNQSLSELVVFWQDQENLVLN
EVYLGKEKFDSVHSKYMNRTSFDSDSWTLRLHNLQIKDKGLYQ
CIIHHKKPTGMIRIHQMNSELSVLANFSQPEIVPISNITENVY
INLTCSSIHGYPEPKKMSVLLRTKNSTIEYDGIMQKSQDNVTE
LYDVSISLSVSFPDVTSNMTIFCILETDKTRLLSSPFSIELED
PQPPPDHIP.

In some cases, a MOD present in a TMP is a FasL polypeptide, e.g., the extracellular domain of a FasL polypeptide. In some cases, a FasL polypeptide of a TMP comprises an amino acid sequence having at least 75%, at least 80%, at least 85%, at least 90%, at least 95%, at least 98%, at least 99%, or 100%, amino acid sequence identity to the following FasL extracellular domain amino acid sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:177).

### Scaffold Polypeptides

A TMP can comprise an Fc polypeptide or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassounch et al. (2012) Methods Enzymol. 502:215; e.g., a polypeptide comprising a pentapeptide repeat unit of (Val-Pro-Gly-X-Gly; SEQ ID NO:193), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; sc, e.g., Megeed et al. (2002) Adv Drug Deli, Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMP, compared to a control TMP lacking the scaffold polypeptide. For example, in some cases, a scaffold polypeptide increases the in vivo half-life (e.g., the serum half-life) of the TMP, compared to a control TMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMP, compared to a control TMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

Fc Polypeptides

In some cases, a TMP comprises an Ig Fc polypeptide. An Ig Fc polypeptide is also referred to herein as an “Fc polypeptide.” The Ig Fc polypeptide of a TMP can be a human IgG1 Fc, a human IgG2 Fc, a human IgG3 Fc, a human IgG4 Fc, etc., or a variant of a wild-type Ig Fc polypeptide. Variants include naturally-occurring variants, non-naturally-occurring variants, and combinations thereof.

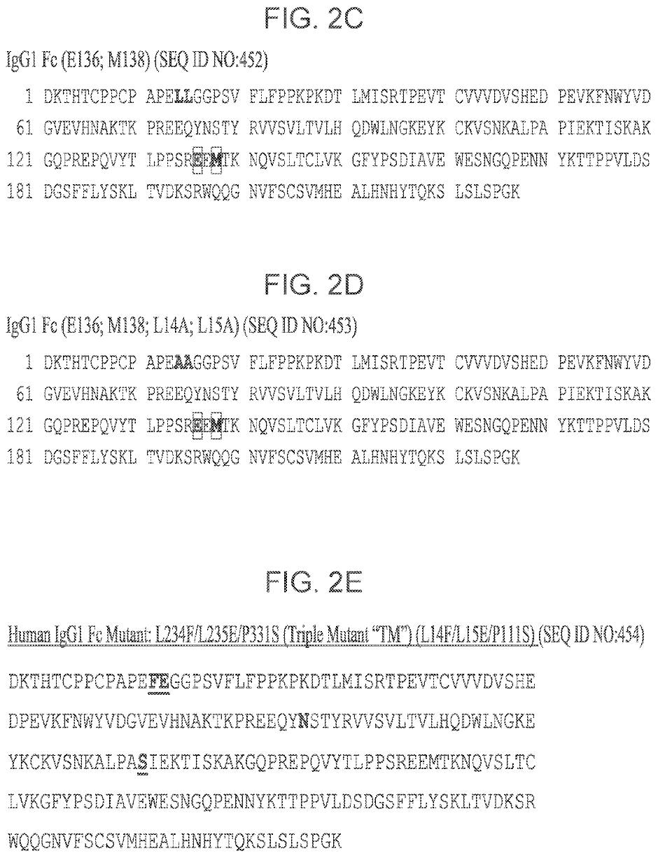

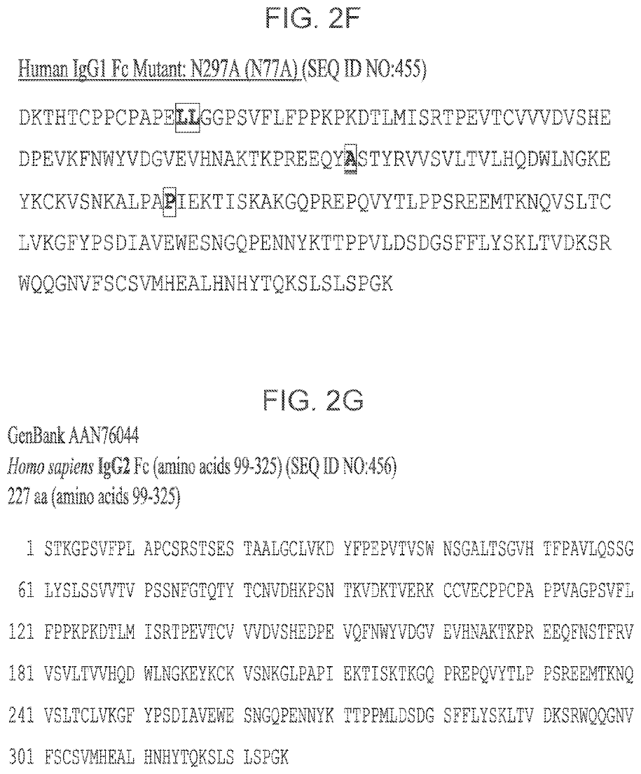

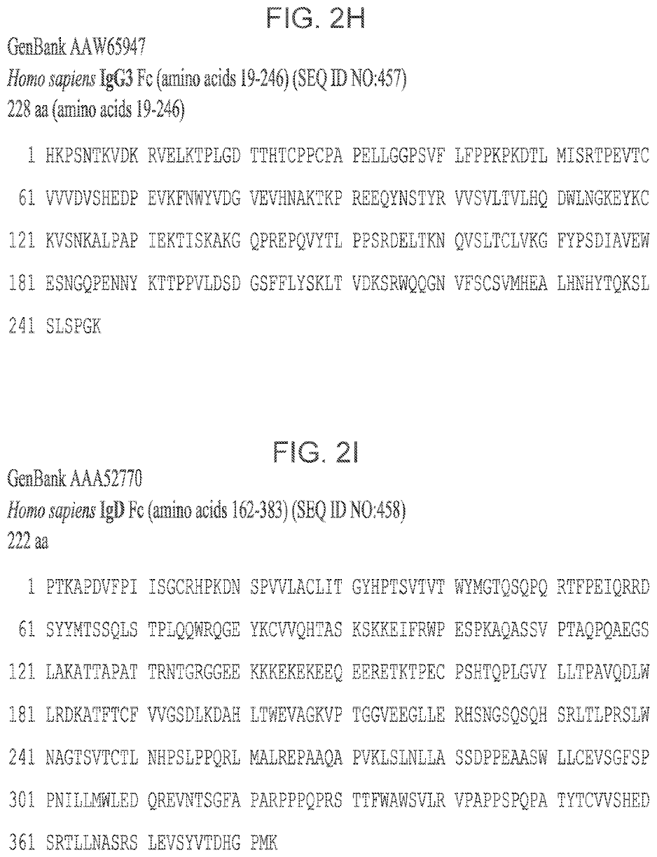

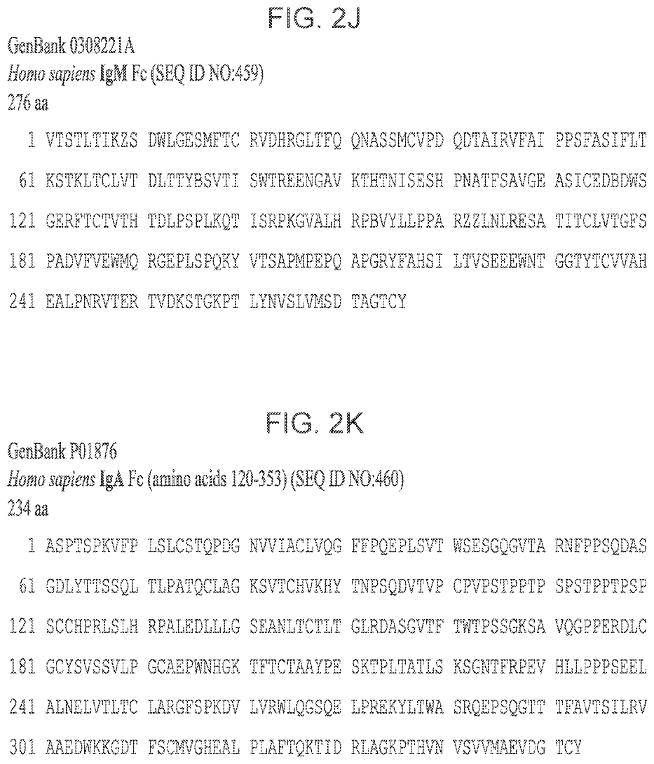

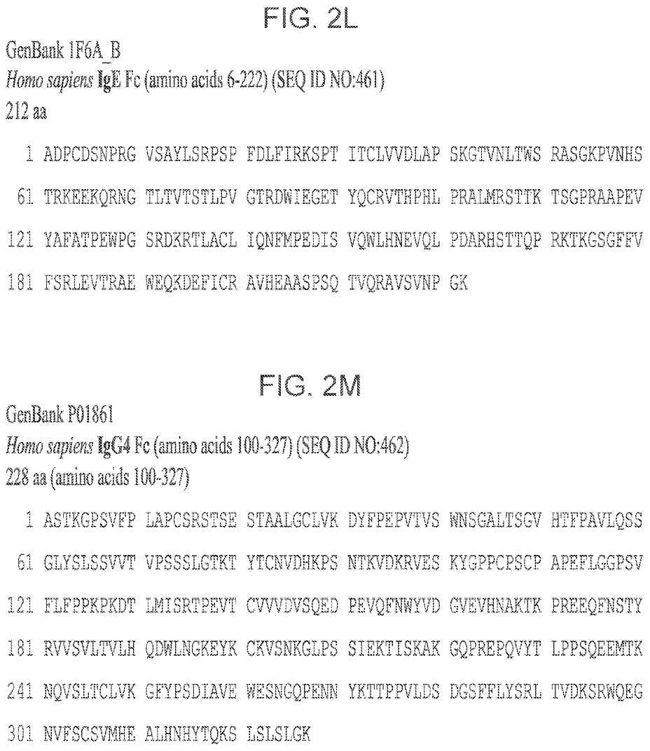

In some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the Fc amino acid sequence depicted in any one of FIG. 2A-2M.

In some cases, the Fc polypeptide present in a TMP is an IgG1 Fc polypeptide, or a variant of an IgG1 Fc polypeptide. For example, in some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 2A. As another example, in some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the Fc polypeptide depicted in FIG. 2B; where the Ig Fc polypeptide comprises an Ala at position 14 and an Ala at position 15. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 2A. In some cases, an Fc polypeptide present in a TMP comprises the amino acid sequence depicted in FIG. 2B.

In some cases, the Fc polypeptide present in a TMP is an IgG1 Fc polypeptide, or a variant of an IgG1 Fc polypeptide, where variants include naturally-occurring variants, non-naturally-occurring variants, and combinations thereof. For example, in some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 2C; where the Ig Fc polypeptide comprises a Glu at position 136 and a Met at position 138. As another example, in some cases, the Fc polypeptide present in a TMP comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 2D; where the Ig Fc polypeptide has Ala at positions 14 and 15; and where the Fc polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 2C. In some cases, an Fc polypeptide present in a TMP comprises the amino acid sequence depicted in FIG. 2D.

In some cases, the Fc polypeptide present in a TMP comprises the amino acid sequence depicted in FIG. 2E (human IgG1 Fc comprising an L234F substitution, an L235E substitution, and a P331S substitution; where L234 corresponds to amino acid 14 of the amino acid sequence depicted in FIG. 31; L235 corresponds to amino acid 15 of the amino acid sequence depicted in FIG. 2E; and P331 corresponds to amino acid 111 of the amino acid sequence depicted in FIG. 2E). In some cases, the Fc polypeptide present in a TMP comprises the amino acid sequence depicted in FIG. 2F, comprising an N279A substitution (N77A of the amino acid sequence depicted in FIG. 2F).

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 2G; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 2G (e.g., where the Ig Fc polypeptide has a length of about 227 amino acids). In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 2H; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 2H (e.g., where the Ig Fc polypeptide has a length of about 228 amino acids). In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 2J; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 2J. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 2K; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 2K.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 2M. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 3M (e.g., where the Ig Fc polypeptide has a length of about 228 amino acids).

In some cases, the IgG4 Fc polypeptide comprises the following amino acid sequence:

(SEQ ID NO: 178)
PPCPSCPAPEFLGGPSVFLFPPKPKDTLMISRTPEVTCVVVD
VSQEDPEVQFNWYVDGVEVHNAKTKPREEQFNSTYRVVSVLT
VLHQDWLNGKEYKCKVSNKGLPSSIEKTISKAKGQPREPQVY
TLPPSQEEMTKNQVSLTCLVKGFYPSDIAVEWESNGQPENNY
KTTPPVLDSDGSFFLYSRLTVDKSRWQEGNVFSCSVMHEALH
NHYTQKSLSLSPG.

In some cases, the Ig Fc employed in a TMP will comprise one or more substitutions of amino acids in the wild-type sequence, such that that Ig Fc that substantially does not induce cell lysis. For example, in some cases the Fc polypeptide present in a TMP comprises the amino acid sequence depicted in FIG. 2A (human IgG1 Fc), except for a substitution of L234 (L14 of the amino acid sequence depicted in FIG. 2A) with an amino acid other than leucine, or a substitution of L235 (L15 of the amino acid sequence depicted in FIG. 3A) with an amino acid other than leucine. Examples include an L234A (L14A) substitution; and an L235A (L15A) substitution.

### Linkers

A TMP can include one or more peptide linkers, i.e., a linker comprising a contiguous stretch of two or more amino acids, where the one or more linkers are between one or more of: i) an MHC class I heavy chain polypeptide and an Ig Fc polypeptide, where such a linker is referred to herein as “L1”; ii) a MOD and an MHC class I polypeptide, where such a linker is referred to herein as “L2”; iii) a first MOD and a second MOD, where such a linker is referred to herein as “L3”; iv) a peptide and an MHC class I polypeptide; and v) a peptide and a β2M polypeptide.

As used herein, the phrase “an optional peptide linker between any two of the components of a TMP” refers to a peptide linker between any two adjacent polypeptides within the TMP. For example, as used herein, the phrase “an optional peptide linker between any two of the components of a TMP” refers to a peptide linker between one or more of: i) a peptide and a β2M polypeptide; ii) a β2M polypeptide and an MHC class I heavy chain polypeptide; iii) an MHC class I heavy chain polypeptide and an Ig Fc polypeptide; iv) an MHC class I heavy chain polypeptide and a MOD; v) an Ig Fc polypeptide and a MOD; and vi) a first MOD and a second MOD. As discussed below, linkers may be a flexible peptide linker, including a short flexible peptide linker, or a rigid peptide linker.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Flexible Peptide Linkers

Exemplary flexible peptide linkers include glycine polymers (G)ₙ, glycine-serine polymers (including, for example, (GS)ₙ, (GSGGS)ₙ (SEQ ID NO:179), (GGGGS)n (SEQ ID NO:180), and (GGGS)ₙ(SEQ ID NO:181), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:182), GGSGG (SEQ ID NO:183), GSGSG (SEQ ID NO:184), GSGGG (SEQ ID NO:185), GGGSG (SEQ ID NO:186), GSSSG (SEQ ID NO:187), and the like.

Exemplary flexible peptide linkers include, e.g., (GGGGS)n (SEQ ID NO:235); also referred to as a “G4S” linker), where n is 1, 2, 3, 4, 5, 6, 7, 8, 9, or 10. In some cases, a linker comprises the amino acid sequence (GGGGS)n (SEQ ID NO:235), where n is 1, 2, 3, 4, 5, 6, 7, 8, 9 or 10. In some cases, a linker comprises the amino acid sequence (GGGGS)n (SEQ ID NO:188), where n is 2. In some cases, a linker comprises the amino acid sequence (GGGGS)n (SEQ ID NO:189), where n is 3. In some cases, a linker comprises the amino acid sequence (GGGWGS)n (SEQ ID NO:190), where n is 4. In some cases, a linker comprises the amino acid sequence (GGGGS)n (SEQ ID NO:191), where n is 7. In some cases, a linker comprises the amino acid sequence AAAGG (SEQ ID NO:192). Also suitable is a linker having the amino acid sequence AAAGG (SEQ ID NO:192). In TMPs of this disclosure, the β2M polypeptide can be connected to the MHC heavy chain polypeptide by a (GGGGS)n (SEQ ID NO:235) linker, where n is 1, 2, 3, 4, 5, 6, 7, 8, 9, or 10, e.g., where n=3, n=4, or n=7.

As used in this disclosure, a “short flexible peptide linker” means a flexible peptide linker that comprises fewer than 15 amino acids. i.e., from 2-14 amino acids. For example, a short flexible peptide linker can comprise from 2-4, 2-5, or 3-6 amino acids (e.g., a GGS linker as discussed in Example 1), or from 4-8, 5-10 or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure. such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:236), A(EAAAK)nA (SEQ ID NO:245), A(EAAAK)nALEA(EAAAK)nA (SEQ ID NO:238), (Lys-Pr)n (SEQ ID NO:242), (Glu-Pro)n (SEQ ID NO:239), (Thr-Pro-Arg)n (SEQ ID NO:240), and (Ala-Pro)n (SEQ ID NO:241) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:194) include EAAAK (SEQ ID NO:194), (EAAAK)₂ (SEQ ID NO:197), (EAAAK), (SEQ ID NO:198). A(EAAAK)₄ALEA(EAAAK)4A (SEQ ID NO:199), and AEAAAKEAAAKA (SEQ ID NO:200). Non-limiting examples of suitable rigid peptide linkers comprising (AP)n include PAPAP (SEQ ID NO:201; also referred to herein as “(AP)2”); APAPAPAP (SEQ ID NO:202; also referred to herein as “(AP)4”); APAPAPAPAPAP (SEQ ID NO:203; also referred to herein as “(AP)6”); APAPAPAPAPAPAPAP (SEQ ID NO:204; also referred to herein as “(AP)8”); and APAPAPAPAPAPAPAPAPAP (SEQ ID NO:205; also referred to herein as “(AP)10”). Non-limiting examples of suitable rigid peptide linkers comprising (KP)n include KPKP (SEQ ID NO:206; also referred to herein as “(KP)2”); KPKPKPKP (SEQ ID NO:207; also referred to herein as “(KP)4”); KPKPKPKPKPKP (SEQ ID NO:208; also referred to herein as “(KP)6”); KPKPKPKPKPKPKPKP (SEQ ID NO:209; also referred to herein as “(KP)8”); and KPKPKPKPKPKPKPKPKPKP (SEQ ID NO:21; also referred to herein as “(KP)10”). Non-limiting examples of suitable rigid peptide linkers comprising (EP)n include EPEP (SEQ ID NO:211; also referred to herein as “(EP)2”); EPEPEPEP (SEQ ID NO:212; also referred to herein as “(EP)4”); EPEPEPEPEPEP (SEQ ID NO:213; also referred to herein as “(EP)6”); EPEPEPEPEPEPEPEP (SEQ ID NO:214; also referred to herein as “(EP)8”); and EPEPEPEPEPEPEPEPEPEP (SEQ ID NO:215; also referred to herein as “(EP)10”).

Generally speaking, a TMP can include a rigid peptide linker and/or short flexible peptide linker between any two of the components of the TMP, but typically, one or more rigid peptide linkers and/or short flexible peptide linkers will be used as follows.
(i) In a TMP comprising one or more Position 2 MODs—between one or more of: i) an MHC class I heavy chain polypeptide and a MOD; ii) a MOD and an Ig Fc polypeptide; and iii) where there are multiple MODs in tandem, between a first MOD and a second MOD.
(ii) In a TMP comprising one or more Position 3 MODs—between one or more of: i) an Ig Fc polypeptide and a MOD; and ii) where there are multiple MODs in tandem, between a first MOD and a second MOD.
(iii) In a TMP comprising one or more Position 4 MODs—between one or more of: i) a KRAS epitope and a MOD; ii) an MHC class I heavy chain polypeptide and an Ig Fc polypeptide; and iii) where there are multiple MODs in tandem, between a first MOD and a second MOD.

Accordingly, this disclosure provides methods of increasing the thermal stability of a TMP comprising one or more MODS in Position 2. Position 3, or Position 4, where the methods comprise interposing a rigid peptide linker or a short flexible linker between two or more of the components of a TMP, as described herein.

As discussed in Example 1 below, it has been found that, in a TMP having one or more Position 3 MODs, the use of a rigid peptide linker or short flexible peptide linker between the Ig Fc polypeptide and a MOD instead of a flexible peptide linker can enhance the thermal stability of the resulting TMP as compared to a TMP that is identical but for a longer, flexible peptide linker such as a (G4S)3 linker (i.e., a (CGGGS)3) linker) (SEQ ID NO:189). While not wishing to be bound by a particular theory, it is believed that the rigid peptide linker or short flexible peptide linker reduces or prevents the interaction of the MOD with other polypeptides within the TMP that can occur with a flexible peptide linker that comprises 15 or more amino acids, resulting in enhanced thermal stability as measured using an accelerated stability assay as described in Example 1.

In some cases, the use of a rigid peptide linker or short flexible peptide linker, when interposed between the Ig Fc polypeptide and a MOD of a TMP having one or more Position 3 MODs, increases thermal stability, as measured by the 37° C. accelerated stability assay described in Example 1, by at least about 10%, at least about 20%, at least about 30%, at least about 40%, at least about 50%, at least about 60%, at least about 70%, at least about 80%, at least about 90%, at least about two-fold, at least about three-fold, at least about four-fold, at least about five-fold, at least about six-fold, at least about seven-fold, at least about eight-fold, or at least about 10-fold, compared to the thermal stability of a control TMP that includes, in place of the rigid peptide linker or short flexible peptide linker, a flexible peptide linker that is a (GGGGS)3 linker (SEQ ID NO:189).

In some cases, the use of a rigid peptide linker or short flexible peptide linker, when interposed between the Ig Fc polypeptide and a MOD of a TMP having one or more Position 3 MODs, increases thermal stability, as measured by the 42° C. accelerated stability assay described in Example 1, by at least about 10%, at least about 20%, at least about 30%, at least about 40%, at least about 50%, at least about 60%, at least about 70%, at least about 80%, at least about 90%, at least about two-fold, at least about three-fold, at least about four-fold, at least about five-fold, at least about six-fold, at least about seven-fold, at least about eight-fold, or at least about 10-fold, compared to the thermal stability of a control TMP that includes, in place of the rigid peptide linker or short flexible peptide linker, a flexible peptide linker that is a (GGGGS)3 linker (SEQ ID NO:189).

### Cysteine-Containing Linkers

As noted above, in some cases, a linker peptide includes a cysteine residue that can form an intrachain disulfide bond with a cysteine residue present elsewhere in the TMP polypeptide chain. For example, as discussed above, in some cases a TMP, or a dimerized TMP such as a homodimer, comprises a linker between the peptide epitope and the β2M polypeptide that includes a cysteine residue that forms an intrachain disulfide bond with a cysteine residue in the MHC class I heavy chain polypeptide present in the TMP. For example, in some cases, where a TMP, or a dimerized TMP such as a homodimer, comprises a cysteine-containing linker between the peptide epitope and the β2M polypeptide, the cysteine residue in the linker forms an intrachain disulfide bond with a cysteine residue at amino acid 236 (e.g., formed by an A236C substitution) in the MHC class I heavy chain polypeptide present in the TMP.

In some cases, the peptide linker between the KRAS peptide and the β2M polypeptide comprises the amino acid sequence GCGGS (SEQ ID NO:216). In some cases, the peptide linker between the KRAS peptide and the β2M polypeptide comprises the amino acid sequence GCGGS(GGGGS)n (SEQ ID NO:217), where n is 1, 2, 3, 4, 5, 6, 7, 8, 9, or 10; e.g., 1, 2, or 3. In some cases, the peptide linker between the peptide and the β2M polypeptide comprises the amino acid sequence GCGGS(GGGGS)n (SEQ ID NO:218), where n is 2.

In some cases, the peptide linker between the KRAS peptide and the β2M polypeptide comprises the amino acid sequence CGGGS (SEQ ID NO:219). In some cases, the peptide linker comprises the amino acid sequence CGGGS(GGGGS)n (SEQ ID NO:220), where n is 1, 2, 3, 4, 5, 6, 7, 8, 9, or 10; e.g., 1, 2, or 3.

In some cases, the peptide linker between the KRAS peptide and the β2M polypeptide comprises the amino acid sequence GGCGS (SEQ ID NO:221). In some cases, the peptide linker comprises the amino acid sequence GGCGS(GGGGS)n (SEQ ID NO:222), where n is 1, 2, 3, 4, 5, 6, 7, 8, 9 or 10, e.g., 1, 2, or 3.

In some cases, the peptide linker between the KRAS peptide and the β2M polypeptide comprises the amino acid sequence GGGCS (SEQ ID NO:223). In some cases, the peptide linker comprises the amino acid sequence GGGCS(GGGGS)n (SEQ ID NO:224), where n is 1, 2, 3, 4, 5, 6, 7, 8, 9, or 10. e.g., 1, 2, or 3.

In some cases, the peptide linker between the KRAS peptide and the β2M polypeptide comprises the amino acid sequence GGGGC (SEQ ID NO:225). In some cases, the peptide linker comprises the amino acid sequence GGGGC(GGGGS)n (SEQ ID NO:226), where n is 1, 2, 3, 4, 5, 6, 7, 8, 9, or 10, e.g., 1, 2, or 3.

### Dimerized TMPs

In some cases, a TMP can form dimers. That is, the present disclosure provides a polypeptide comprising a dimer of two TMPs. The present disclosure thus provides a protein that is a dimerized TMP comprising two TMPs that are covalently linked to each other. The covalent linkage of the dimer can be one or more disulfide bonds between an Ig Fc polypeptide in the first TMP and an Ig Fc polypeptide in the second TMP. As but one example, the Ig Fc can be a variant of a human IgG1 Fc polypeptide, which variant has a substantially reduced ability to effect complement-dependent cytotoxicity (CDC) or antibody-dependent cell cytotoxicity (ADCC) (e.g., the human IgG1 Fc polypeptide of FIG. 2B or FIG. 2D). When the TMP comprises an Ig Fc polypeptide, the TMP typically will self-assemble into a dimer by spontaneously forming disulfide bonds with the IgG1 Fc polypeptide of another TMP. Thus, e.g., the Ig Fc polypeptides in the first TMP and the second TMP can be linked to one another by one or more disulfide bonds. In many cases, the two TMPs will be identical to one another in amino acid sequence and comprise Ig Fc polypeptides that spontaneously form one or more disulfide bonds, thereby forming a dimerized TMP that is a homodimer.

Accordingly, the present disclosure provides a protein comprising: a) a first TMP; and b) a second TMP, which optionally may be identical to the first TMP, where the first and second TMPs are covalently linked to one another. The covalent linkage can be a disulfide bond between an Ig Fc polypeptide in the first TMP and an Ig Fc polypeptide in the second TMP.

If desired, the Ig Fc polypeptides of each TMP can comprise interspecific dimerization sequences, e.g., “Knob-in-Hole” sequences that permit two different TMPs to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. Sec Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Thus, in some cases, a dimerized TMP can be a heterodimer, comprising two TMP chains that are not identical in amino acid sequence.

Interspecific dimerization sequences also may be employed to enable TMPs to be linked to non-TMP molecules that can provide additional functionality to the TMP. For example, a TMP could be linked to a molecule that comprise polypeptides (e.g., antibodies or binding fragments thereof such as scFvs) that bind to cancer-associated antigens, thereby enabling the TMP to localize to tissues comprising the cancer-associated antigen.

### Additional Polypeptides

A polypeptide chain of a TMP can include one or more polypeptides and conjugate drugs in addition to those described above. Suitable additional polypeptides, including epitope tags and affinity domains, and drug conjugates are described in in published PCT applications WO2020132138A1 and WO2019/051091, discussed above, the disclosures of which as they pertain to epitope tags, affinity domains and drug conjugates are expressly incorporated herein by reference, including specifically paragraphs [00498]-[00508] of WO2020132138A1 and paragraphs [00353]-[00363] of WO2019/051091. The one or more additional polypeptides can be included at the N-terminus of the TMP polypeptide chain, at the C-terminus of the TMP polypeptide chain, or internally within the polypeptide chain of a TMP. As discussed above, additional polypeptides also could be conjugated to TMPs through the use of interspecific sequences.

### Exemplary TMPs and Dimerized TMPs

In the discussion below, the discussion of exemplary TMPs is intended to encompass both TMPs and dimerized TMPs comprising two TMPs where the TMPs are joined by one or more covalent bonds that join the two TMPs. e.g., one or more disulfide bonds that spontaneously form between the Ig Fc polypeptides in the two TMPs. Such dimerized TMPs can be either i) homodimers comprising two TMPs, where both of the TMPs have the same amino acid sequence, or ii) heterodimers comprising two TMPs, where the two TMPs differ from one another in amino acid sequence.

In some cases, a TMP comprises: i) a KRAS peptide; ii) a first MHC class I polypeptide, where the first MHC class I polypeptide is a β2M polypeptide; iii) a second MHC class I polypeptide, where the second MHC class I polypeptide is an MHC class I heavy chain polypeptide; iv) at least one MOD; and v) an Ig Fc polypeptide. The TMP may comprise one or more peptide linkers between one or more of the components.

In the case of a TMP comprising one or more Position 2 MODs. one or more peptide linkers may be interposed between: i) the KRAS peptide and the β2M polypeptide; ii) the β2M polypeptide and the MHC class I heavy chain polypeptide; iii) between the MHC class I heavy chain polypeptide and a MOD; iv) between a MOD and the Ig Fc polypeptide and the MOD; and vi) where the TMP comprises two or more MODs in tandem, between the MODs. As discussed above, in such TMPs, a rigid peptide linker or short flexible peptide linker may be interposed between one or more of: i) an MHC class I heavy chain polypeptide and a MOD; ii) a MOD and an Ig Fc polypeptide; and iii) where there are multiple MODs in tandem, between a first MOD and a second MOD, and so on for additional MODs in tandem. In some cases, the rigid peptide linker comprises an amino acid sequence selected from EAAAK (SEQ ID NO:194), A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). In some cases, the short flexible peptide linkers will comprise from 2-4, 2-5, 3-6, 4-8, 5-10 or 10-14 amino acids. In some cases, the short flexible peptide linker is GGS. Generally speaking, flexible peptide linkers will be interposed between the components that are not connected by a rigid peptide linker or short flexible peptide linker, wherein the linker between the KRAS peptide and β2M polypeptide further may comprise a Cys-containing linker as discussed above.

In the case of a TMP comprising one or more Position 3 MODs, one or more peptide linkers may be interposed between: i) the KRAS peptide and the β2M polypeptide; ii) the β2M polypeptide and the MHC class I heavy chain polypeptide; iii) the MHC class I heavy chain polypeptide and an Ig Fc polypeptide; iv) the Ig Fc polypeptide and the MOD; and v) where the TMP comprises two or more MODs in tandem, between the MODs. As discussed above, in such TMPs, a rigid peptide linker or short flexible peptide linker may be interposed between one or more of: i) an Ig Fc polypeptide and a MOD; and ii) where there are multiple MODs in tandem, between one or more of the MODs, e.g., between a first MOD and a second MOD when there are two MODs in tandem. In some cases, the rigid peptide linker comprises an amino acid sequence selected from EAAAK (SEQ ID NO:194), A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). In some cases, the short flexible peptide linkers will comprise from 2-4, 2-5, 3-6, 4-8, 5-10 or 10-14 amino acids. In some cases, the short flexible peptide linker is GGS. Generally speaking, flexible peptide linkers will be interposed between the components that are not connected by a rigid peptide linker or short flexible peptide linker, wherein the linker between the KRAS peptide and β2M polypeptide further may comprise a Cys-containing linker as discussed above.

As noted above, in some cases the at least one MOD present in the TMP is a wild-type MOD. In other cases, the at least one MOD present in the TMP is a variant MOD that exhibits reduced affinity for a co-MOD, compared to the affinity of a corresponding wild-type MOD for the co-MOD.

In any of the above TMPs, the KRAS peptide has an amino acid sequence selected from any of the KRAS peptide sequences described above. For examples, the KRAS peptide may have from the following group: VVGADGVGK (SEQ ID NO:108); VVGACGVGK (SEQ ID NO:109); VVGAVGVGK (SEQ ID NO:110); VVVGADGVGK (SEQ ID NO:111); VVVGAVGVGK (SEQ ID NO:112); VVVGACGVGK (SEQ ID NO:113); VTGADGVGK (SEQ ID NO:114); VTGAVGVGK (SEQ ID NO: 115); VTGACGVGK (SEQ ID NO: 116); VTVGADGVGK (SEQ ID NO:117); VTVGAVGVGK (SEQ ID NO: 118); VTVGACGVGK (SEQ ID NO: 119); LVVVGADGV (SEQ ID NO:124); LVVVGAVGV (SEQ ID NO:125); LVVVGACGV (SEQ ID NO:126); KLVVVGADGV (SEQ ID NO:127); KLVVVGAVGV (SEQ ID NO:128); KLVVVGACGV (SEQ ID NO:129); LLVVGADGV (SEQ ID NO:130); LLVVGAVGV (SEQ ID NO:131); LLVVGACGV (SEQ ID NO:132); FLVVVGADGV (SEQ ID NO:133); FLVVVGAVGV (SEQ ID NO:134); FLVVVGACGV (SEQ ID NO: 135).

In the above TMPs, in some cases, the second MHC polypeptide is an HLA heavy chain that comprises an amino acid sequence having at least 75%, at least 80%, at least 85%, at least 90%, at least 95%, at least 98%, at least 99%, or 100% amino acid sequence identity to an HLA-A*0201 polypeptide, an HLA-A*1101 polypeptide, or HLA-A24 polypeptide, HLA-E polypeptide such as HLA-E*0101 or HLA-E*01.03, or an HLA-G polypeptide such as HLA-G*0101 or and HLA-G*01:04. In some cases, the HLA heavy chain polypeptide is an HLA-A*0201 polypeptide. In some cases, the HLA heavy chain polypeptide is an HLA-A*0201 polypeptide comprising a Y84C substitution and/or an A236C substitution. In some cases, the HLA heavy chain polypeptide is an HLA-A*1101 polypeptide comprising a Y84C substitution and/or an A236C substitution. In some cases, the HLA heavy chain polypeptide is an HLA-E*0101 or HLA-E*01.03*0201 polypeptide comprising a Y84C substitution and/or an A236C substitution. In some cases, the HLA heavy chain polypeptide is an HLA-G*0101 or and HLA-G*01:04A*1101 polypeptide comprising a Y84C substitution and/or an A236C substitution.

In some cases, a IMP comprises two MODs, where the two MODs have the same amino acid sequence. e.g., the MOD is a variant IL-2 polypeptide that exhibits reduced binding affinity for both the α and β chains of IL2R as compared to wt. IL-2 having a sequence of FIG. 17A, e.g., a variant IL-2 polypeptide comprising H16A and F42A substitutions, or a variant IL-2 polypeptide comprising H16T and F42A substitutions.

In some cases, the Ig Fc polypeptide is a variant of a human IgG1 Fc polypeptide that substantially does not induce cell lysis, e.g., an IgG1 Fc polypeptide comprising L234A and L235A substitutions such as is shown in FIG. 2B or FIG. 2D.

In some cases, a TMP comprises a MOD at Position 3, wherein the HLA heavy chain polypeptide is a wild-type or variant HLA-A*0201 polypeptide, e.g., an HLA-A*0201 polypeptide comprising at least 80%, at least 85%, at least 90%, at least 95%, at least 98%, at least 99%, or 100% amino acid sequence identity to a HLA-A*0201 polypeptide of one of FIG. 3A, 3B, 3C, 3D or 3E, optionally comprising a Y84 substitution and/or an A236C substitution. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 2B or FIG. 2D. In some cases, the MOD is a variant IL-2 polypeptide that exhibits reduced binding affinity for both the α and β chains of IL2R as compared to wt. IL-2 having a sequence of FIG. 17A, e.g., a variant IL-2 polypeptide comprising H16A and F42A substitutions, or a variant IL-2 polypeptide comprising H16T and F42A substitutions. In some cases, the KRAS peptide has an amino acid sequence selected from any of the KRAS peptide sequences described above. For examples, the KRAS peptide may have from the following group: VVGADGVGK (SEQ ID NO:108); VVGACGVGK (SEQ ID NO:109); VVGAVGVGK (SEQ ID NO:110); VVVGADGVGK (SEQ ID NO:111); VVVGAVGVGK (SEQ ID NO:112); VVVGACGVGK (SEQ ID NO:113); VTGADGVGK (SEQ ID NO:114); VTGAVGVGK (SEQ ID NO: 115); VTGACGVGK (SEQ ID NO: 116); VTVGADGVGK (SEQ ID NO: 117); VTVGAVGVGK (SEQ ID NO: 118); VTVGACGVGK (SEQ ID NO: 119); LVVVGADGV (SEQ ID NO:124); LVVVGAVGV (SEQ ID NO:125); LVVVGACGV (SEQ ID NO:126); KLVVVGADGV (SEQ ID NO:127); KLVVVGAVGV (SEQ ID NO:128); KLVVVGACGV (SEQ ID NO:129); LLVVGADGV (SEQ ID NO:130); LLVVGAVGV (SEQ ID NO:131); LLVVGACGV (SEQ ID NO:132); FLVVVGADGV (SEQ ID NO:133); FLVVVGAVGV (SEQ ID NO:134); FLVVVGACGV (SEQ ID NO:135). In such cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:194). A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). Alternatively, in some cases, the TMP comprises a short flexible peptide linker between a variant IL-2 MOD and an Ig Fc polypeptide present in the TMP, where the short flexible peptide linker comprises from 2-4, 2-5, 3-6, 4-8, 5-10 or 10-14 amino acids. In some cases, the short flexible peptide linker is GGS. In some cases, the TMP comprises two copies of the variant IL-2 MOD. In some cases, the TMP comprises two copies of the variant IL-2 MOD, and the TMP comprises a rigid peptide linker between: a) a first variant IL-2 MOD and an Ig Fc polypeptide present in the TMP; and b) between the first variant IL-2 MOD and the second variant IL-2 MOD, where the rigid peptide linker comprises an amino acid sequence selected from EAAAK (SEQ ID NO:194), A(EAAAK)n (SEQ ID NO:244), A(EAAAK)nA (SEQ ID NO:245). (AP)n (SEQ ID NO:246). (EP)n (SEQ ID NO:247), and (KP)n (SEQ ID NO:248), where n is an integer from 1 to 10 (e.g., n is 1, 2, 3, 4, 5, 6, 7, 8, 9, or 10).

In some cases, a TMP comprises a MOD at Position 3, wherein the HLA heavy chain polypeptide is a wild-type or variant HLA-A*1101 polypeptide, e.g., an HLA-A*1101 polypeptide comprising at least 80%, at least 85%, at least 90%, at least 95%, at least 98%, at least 99%, or 100° % amino acid sequence identity to a HLA-A*1101 polypeptide of one of FIG. 4A, 4B, 4C, 4D or 4E, optionally comprising a Y84 substitution and/or an A236C substitution. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 2B or FIG. 2D. In some cases, the MOD is a variant IL-2 polypeptide that exhibits reduced binding affinity for both the α and β chains of IL2R as compared to wt. IL-2 having a sequence of FIG. 17A, e.g., a variant IL-2 polypeptide comprising H16A and F42A substitutions, or a variant IL-2 polypeptide comprising H16T and F42A substitutions. In some cases, the KRAS peptide has an amino acid sequence selected from any of the KRAS peptide sequences described above. For examples, the KRAS peptide may have from the following group: VVGADGVGK (SEQ ID NO:108); VVGACGVGK (SEQ ID NO:109); VVGAVGVGK (SEQ ID NO:110); VVVGADGCVGK (SEQ ID NO:111); VVVGAVGVGK (SEQ ID NO:112); VVVGACGVGK (SEQ ID NO:113); VTGADGVGK (SEQ ID NO: 114); VTGAVGVGK (SEQ ID NO: 115); VTGACGVGK (SEQ ID NO: 116); VTVGADGVGK (SEQ ID NO: 117); VTVGAVGVGK (SEQ ID NO: 118); VTVGACGVGK (SEQ ID NO: 119); LVVVGADGV (SEQ ID NO:124); LVVVGAVGV (SEQ ID NO:125); LVVGACGV (SEQ ID NO:126); KLVVVGADGV (SEQ ID NO:127); KLVVVGAVGV (SEQ ID NO:128); KLVVVGACGV (SEQ ID NO:129); LLVVGADGV (SEQ ID NO:130); LLVVGAVGV (SEQ ID NO:131); LLVVGACGV (SEQ ID NO:132); FLVVVGADGV (SEQ ID NO:133); F1LVVVGAVGV (SEQ ID NO:134); FLVVVGACGV (SEQ ID NO:135). In such cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:194), A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). In some cases, the TMP comprises two copies of the variant IL-2 MOD. In some cases, the TMP comprises two copies of the variant IL-2 MOD, and the TMP comprises a rigid peptide linker between: a) a first variant IL-2 MOD and an Ig Fc polypeptide present in the TMP; and b) between the first variant IL-2 MOD and the second variant IL-2 MOD, where the rigid peptide linker comprises an amino acid sequence selected from EAAAK (SEQ ID NO:194). A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). Alternatively, in some cases, the TMP comprises a short flexible peptide linker between a variant IL-2 MOD and an Ig Fc polypeptide present in the TMP, where the short flexible peptide linker comprises from 2-4, 2-5, 3-6, 4-8, 5-10 or 10-14 amino acids. In some cases, the short flexible peptide linker is GGS.

In some cases, a TMP comprises a MOD at Position 3, wherein the HLA heavy chain polypeptide is a wild-type or variant HLA-A*2402 polypeptide, e.g., an HLA-A*2402 polypeptide comprising at least 80%, at least 85%, at least 90%, at least 95%, at least 98%, at least 99%, or 100% amino acid sequence identity to a HLA-A*2402 polypeptide of one of FIG. 5A, 5B, 5C, 5D or 5E, optionally comprising a Y84 substitution and/or an A236C substitution. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 2B or FIG. 2D. In some cases, the MOD is a variant IL-2 polypeptide that exhibits reduced binding affinity for both the α and β chains of IL2R as compared to wt. IL-2 having a sequence of FIG. 17A, e.g., a variant IL-2 polypeptide comprising H16A and F42A substitutions, or a variant IL-2 polypeptide comprising H16T and F42A substitutions. In some cases, the KRAS peptide has an amino acid sequence selected from any of the KRAS peptide sequences described above. For examples, the KRAS peptide may have from the following group: VVGADGVGK (SEQ ID NO:108); VVGACGVGK (SEQ ID NO:109); VVGAVGVGK (SEQ ID NO:110); VVVGADGVGK (SEQ ID NO:111); VVVGAVGVGK (SEQ ID NO:112); VVVGACGVGK (SEQ ID NO:113); VTGADGVGK (SEQ ID NO:114); VTGAVGVGK (SEQ ID NO:115); VTGACGVGK (SEQ ID NO: 116); VTVGADGVGK (SEQ ID NO:117); VTVGAVGVGK (SEQ ID NO:118); VTVGACGVGK (SEQ ID NO: 119); LVVVGADGV (SEQ ID NO:124); LVVVGAVGV (SEQ ID NO:125); LVVVGACGV (SEQ ID NO:126); KLVVVGADGV (SEQ ID NO:127); KLVVVGAVGV (SEQ ID NO:128); KLVVVGACGV (SEQ ID NO:129); LLVVGADGV (SEQ ID NO:130); LLVVGAVGV (SEQ ID NO:131); LLVVGACGV (SEQ ID NO:132); FLVVVGADGV (SEQ ID NO:133); FLVVVGAVGV (SEQ ID NO:134); FLVVVGACGV (SEQ ID NO:135). In such cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:194). A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). In some cases, the TMP comprises two copies of the variant IL-2 MOD. In some cases, the TMP comprises two copies of the variant IL-2 MOD, and the TMP comprises a rigid peptide linker between: a) a first variant IL-2 MOD and an Ig Fc polypeptide present in the TMP; and b) between the first variant IL-2 MOD and the second variant IL-2 MOD, where the rigid peptide linker comprises an amino acid sequence selected from EAAAK (SEQ ID NO:194), A(EAAAK)n (SEQ ID NO:244), A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). Alternatively, in some cases, the TMP comprises a short flexible peptide linker between a variant IL-2 MOD and an Ig Fc polypeptide present in the TMP, where the short flexible peptide linker comprises from 2-4, 2-5, 3-6, 4-8, 5-10 or 10-14 amino acids. In some cases, the short flexible peptide linker is GGS.

As discussed above, a TMP can be include one or more intrachain disulfide bonds. For example, a TMP can comprise a β2M polypeptide having an R12C substitution and a class 1 MHC heavy chain polypeptide having an A236C substitution; such that a disulfide bond forms between the Cys at position 12 of the β2M polypeptide and the Cys at position 236 of the class I MHC heavy chain polypeptide. As another example, a TMP can comprise i) a KRAS epitope and a β2M polypeptide that are joined by a peptide linker comprising a GCGGS(GGGGS)ₙ (SEQ ID NO:249) sequence, where n is 1, 2, or 3, and ii) a class I MHC heavy chain polypeptide having a Y84C substitution. such that a disulfide bond forms between the Cys in the peptide linker and the Cys at position 84 of the class I MHC heavy chain polypeptide. In other examples, a TMP can comprise i) a KRAS epitope and a β2M polypeptide that are joined by a peptide linker comprising a GCGGS(GGGGS)ᵣ (SEQ ID NO:249) sequence, where n is 1, 2, or 3, and where the β2M polypeptide comprises an R12C substitution; and ii) a class I MHC heavy chain polypeptide having a Y84C substitution and an A236C substitution. such that a) a first disulfide bond forms between the Cys in the peptide linker and the Cys at position 84 of the class I MHC heavy chain polypeptide, and b) a second disulfide bond forms between the Cys at position 12 of the β2M polypeptide and the Cys at position 236 of the class I MHC heavy chain polypeptide. For simplicity, the first disulfide bond is referred to as “G2C/Y84C”; and the second disulfide bond is referred to as “R12C/A236C.” A TMP can include: a) a G2C/Y84C disulfide bond and not an R12C/A236C disulfide bond; b) an R12C/A236C disulfide bond and not a G2C/Y84C disulfide bond; or c) a G2C/Y84C disulfide bond and an R12C/A236C disulfide bond. In some cases, the MHC class I heavy chain comprises a non-naturally occurring Cys at position 84 and a non-naturally occurring residue at position 139, such that an intrachain disulfide bond forms between the Cys-84 and the Cys-139.

A TMP can include: a) a G2C/Y84C disulfide bond and not an R12C/A236C disulfide bond; and b) at least one MOD at position 2 or 3. A TMP can include: a) an R12C/A236C disulfide bond and not a G2C/Y84C disulfide bond; and at least one MOD at position 2 or 3. A TMP can include: a) a G2C/Y84C disulfide bond and an R12C/A236C disulfide bond; and b) and at least one MOD at position 2 or 3.

In some cases, a TMP comprises an MHC class I heavy chain polypeptide comprising (i) an HLA-A0201 (Y84A; A236C) polypeptide comprising an Ala at position 84 and a Cys at position 236, or (ii) an HLA-A0201 (Y84C; A139C) polypeptide comprising a Cys at positions 84 and 139, or (iii) an HLA-A0201 (Y84C; A236) polypeptide comprising a Cys at position 84 and an alanine at position 236; where examples are depicted in FIG. 31-3E.

In some cases, a TMP comprises an MHC class I heavy chain polypeptide comprising (i) an HLA-A*1101 (Y84A; A236C) polypeptide comprising an Ala at position 84 and a Cys at position 236, or (ii) an HLA-A*1101 (Y84C; A236C) polypeptide comprising a Cys at positions 84 and 236, or (iii) an HLA-A*1101 (Y84C; A236) polypeptide comprising a Cys at position 84 and an alanine at position 236; where examples are depicted in FIG. 4B-4E.

In some cases, a TMP comprises an MHC class I heavy chain comprising (i) an HLA-A24 (Y84A; A236C polypeptide comprising an Ala at position 84 and a Cys at position 236, or (ii) an HLA-A24 (Y84C; A236C) polypeptide comprising a Cys at positions 84 and 236, or (iii) an HLA-A24 (Y84C; A236) polypeptide comprising a Cys at position 84 and an alanine at position 236; where examples are depicted in FIG. 5B-5E.

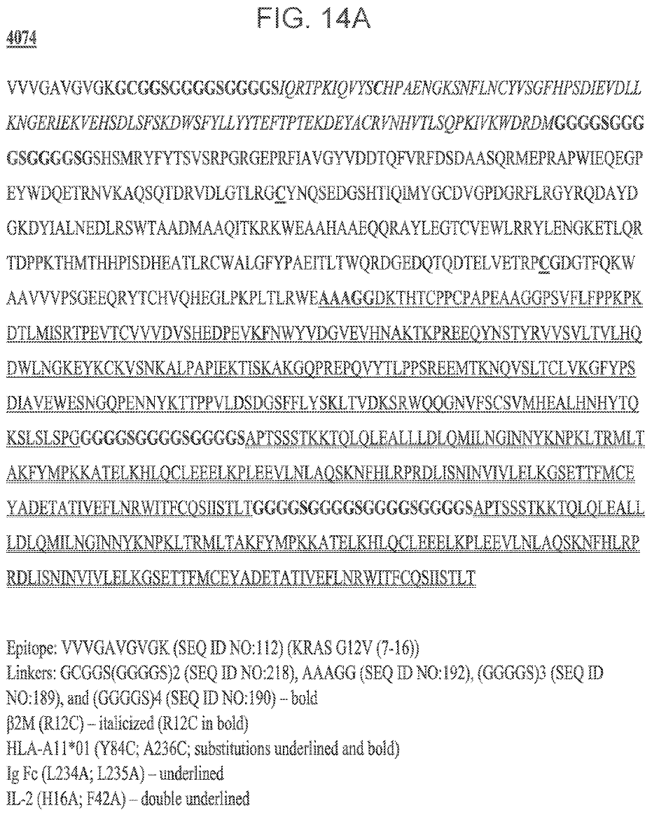

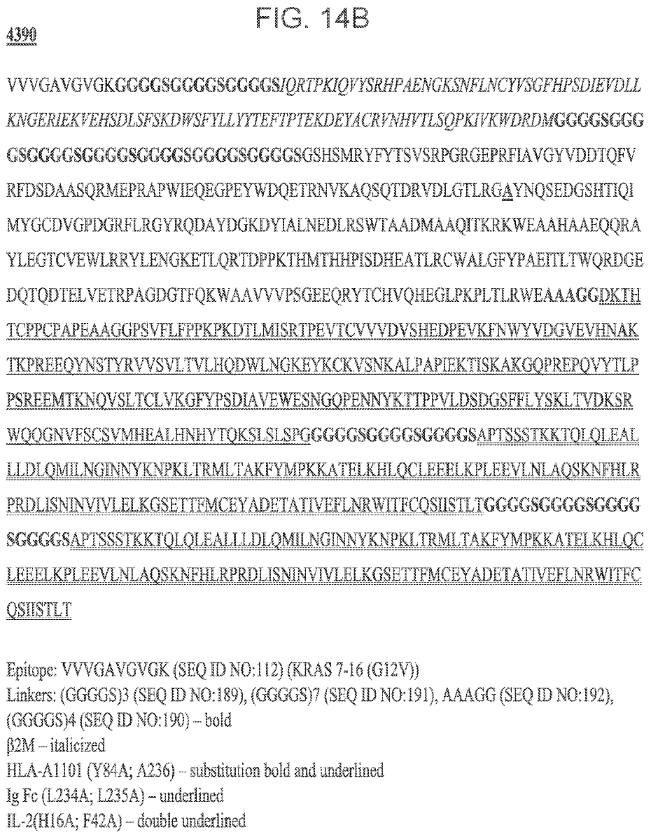

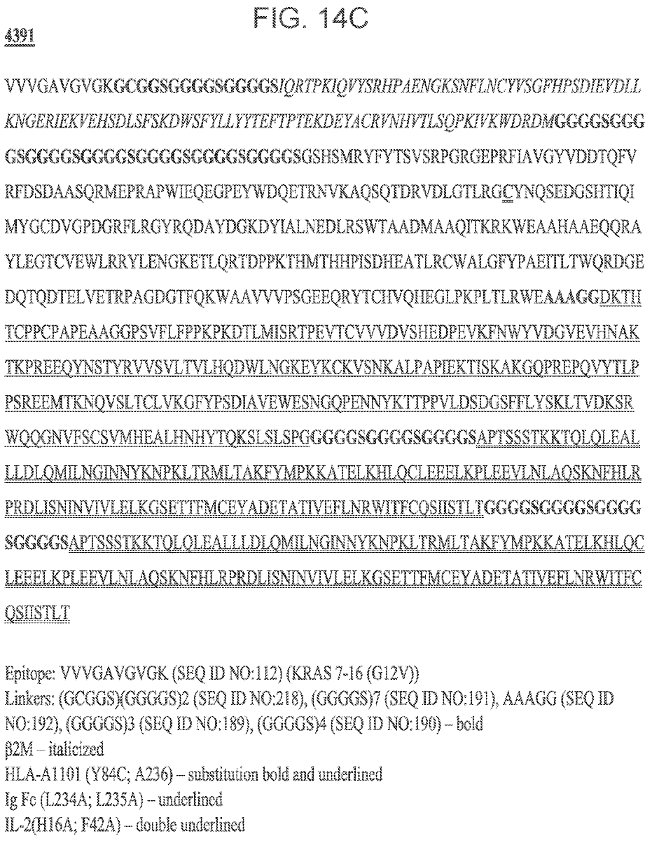

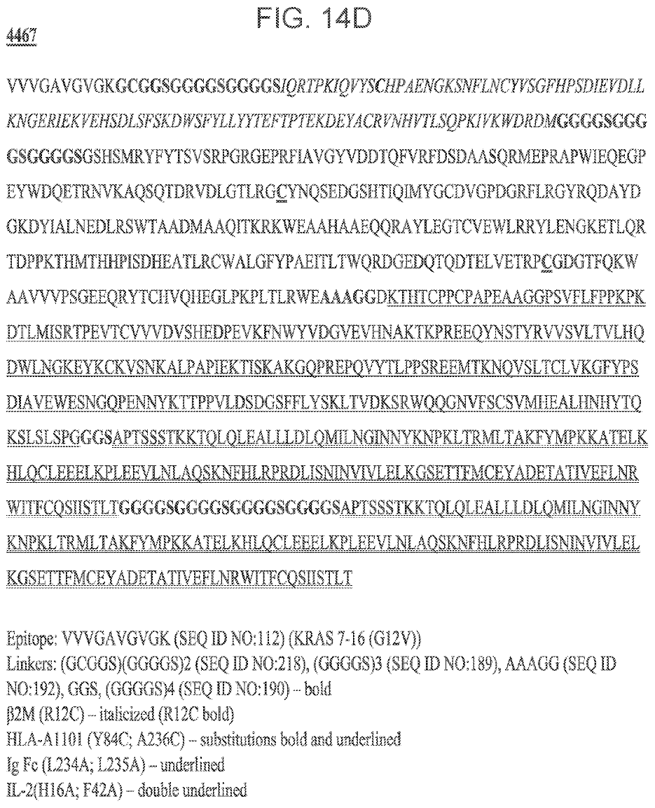

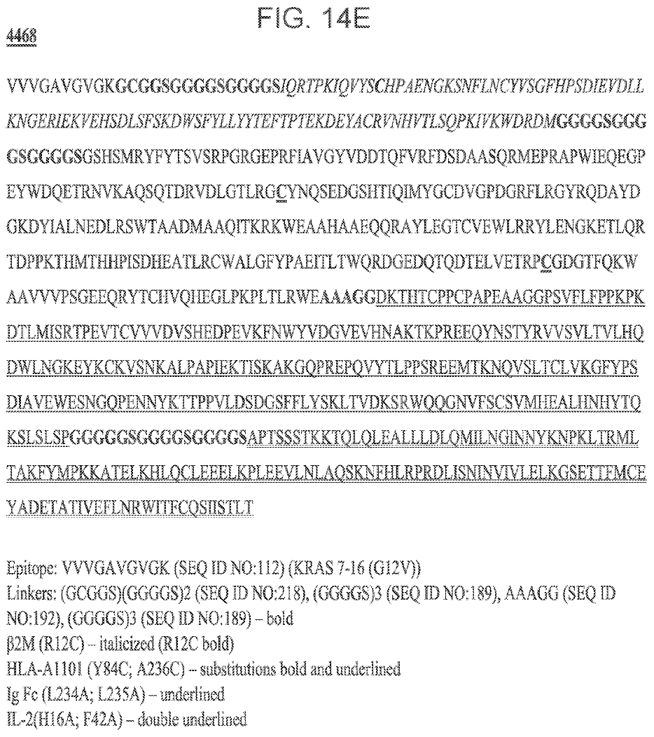

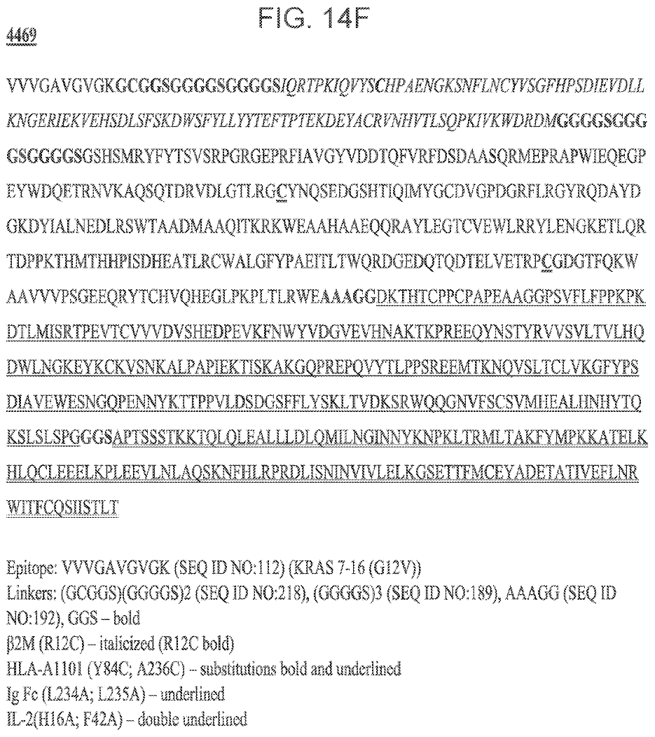

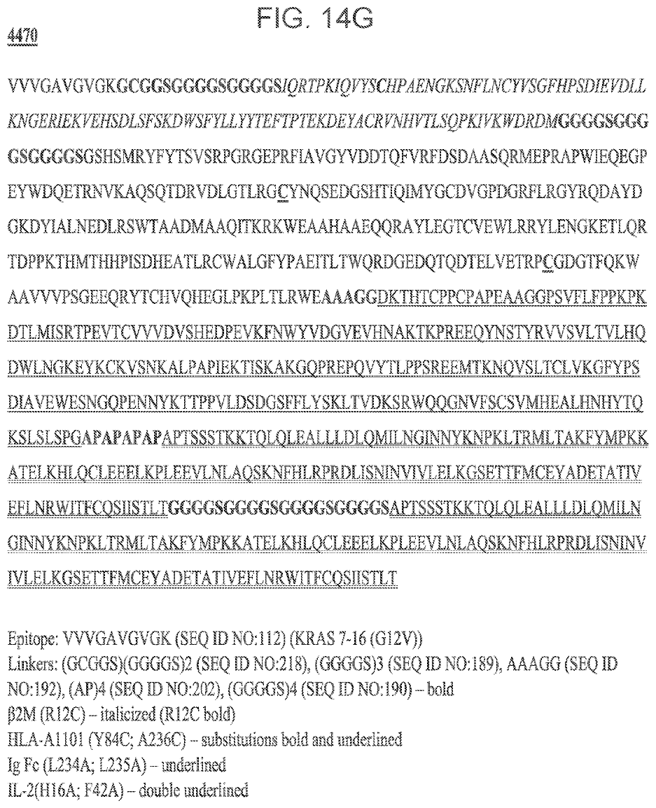

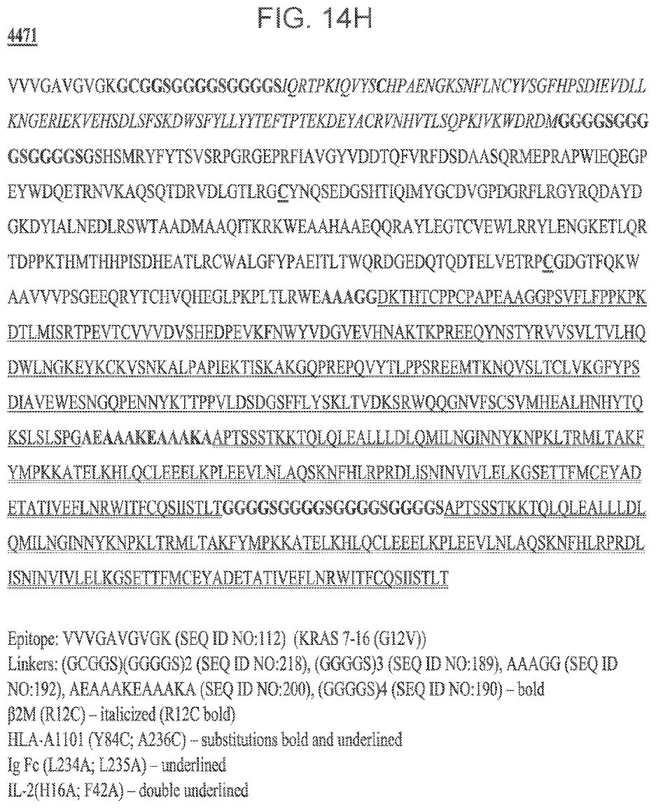

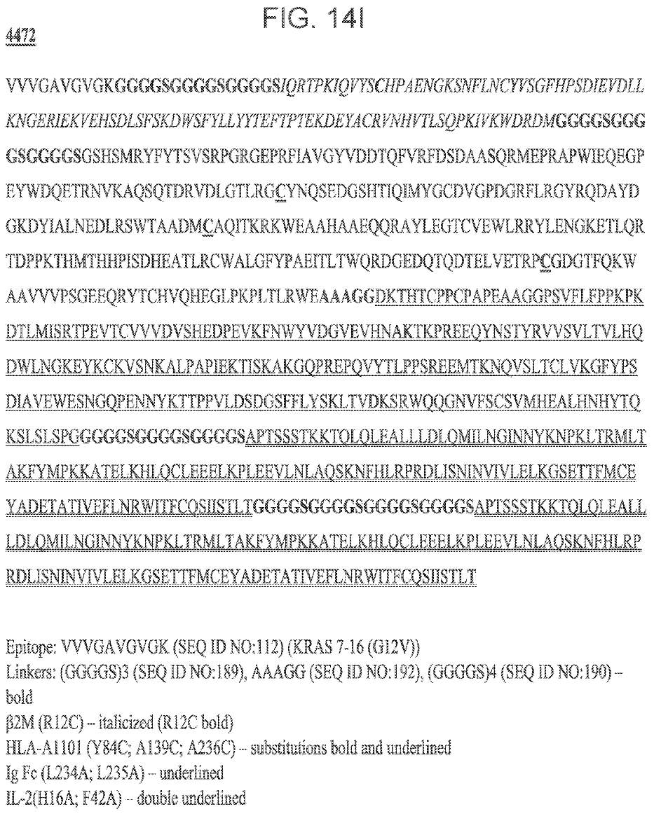

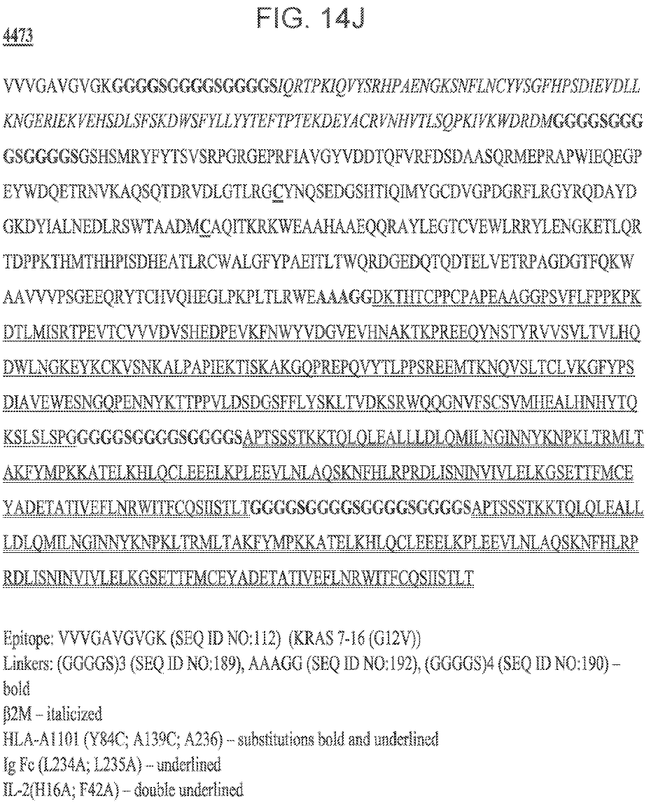

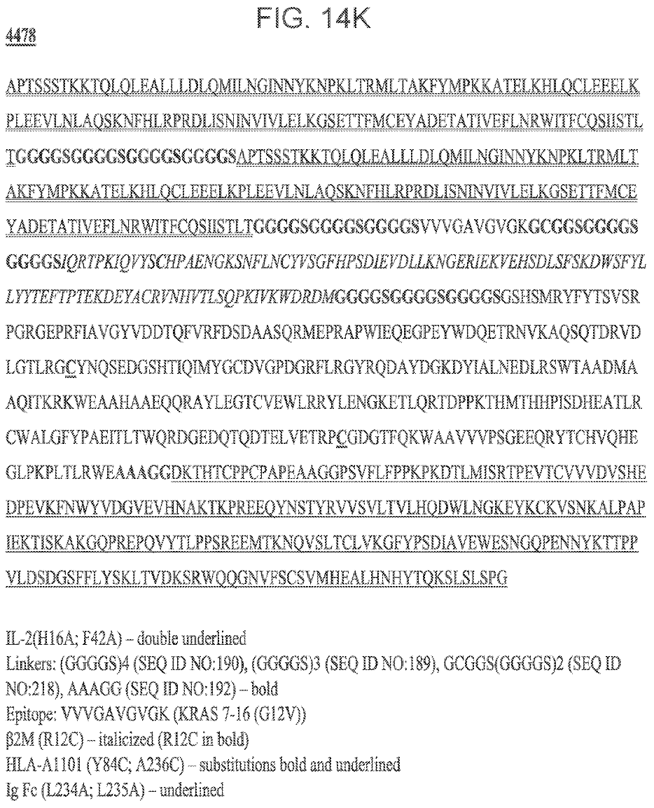

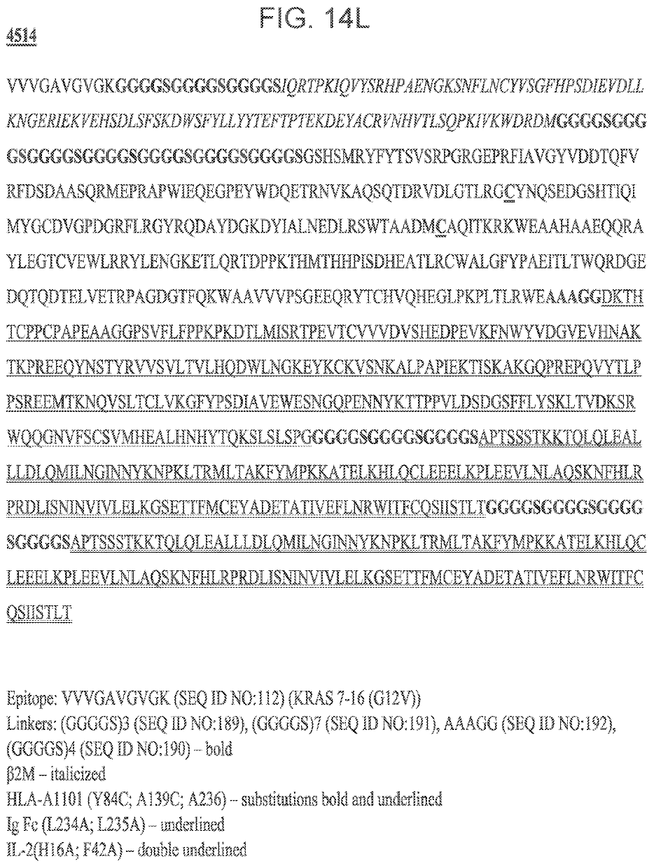

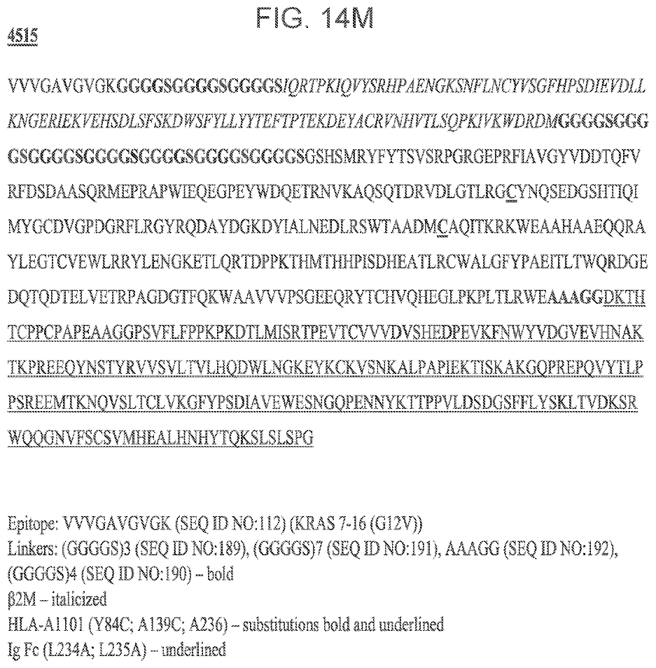

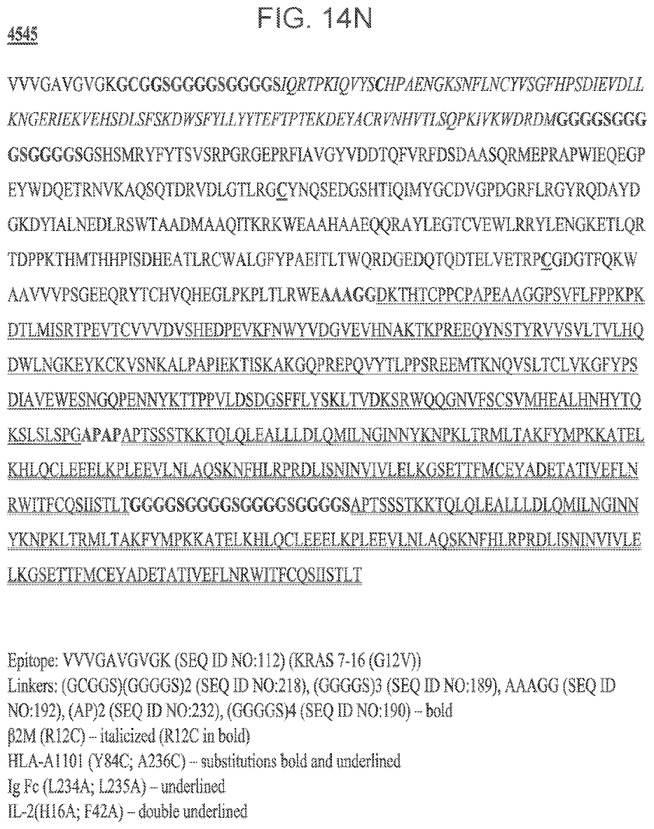

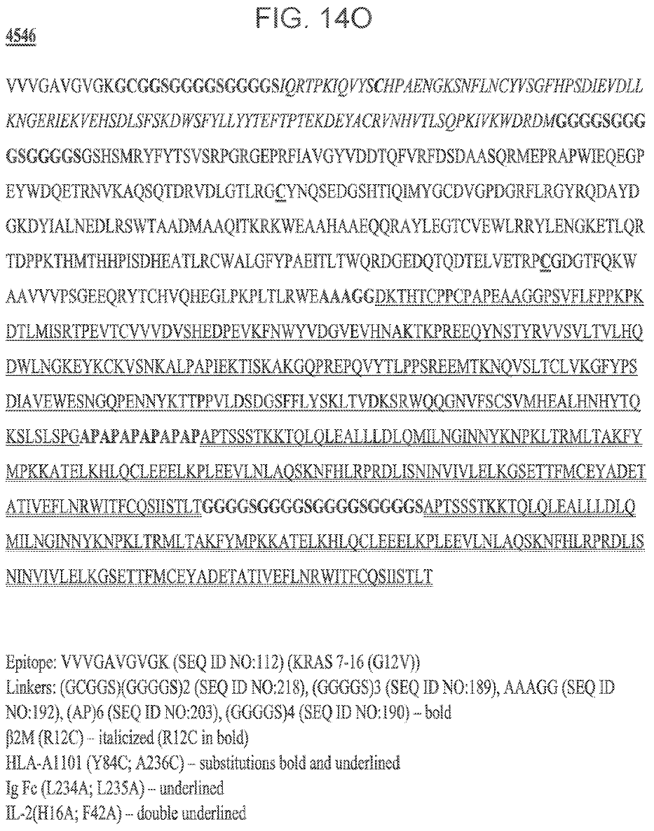

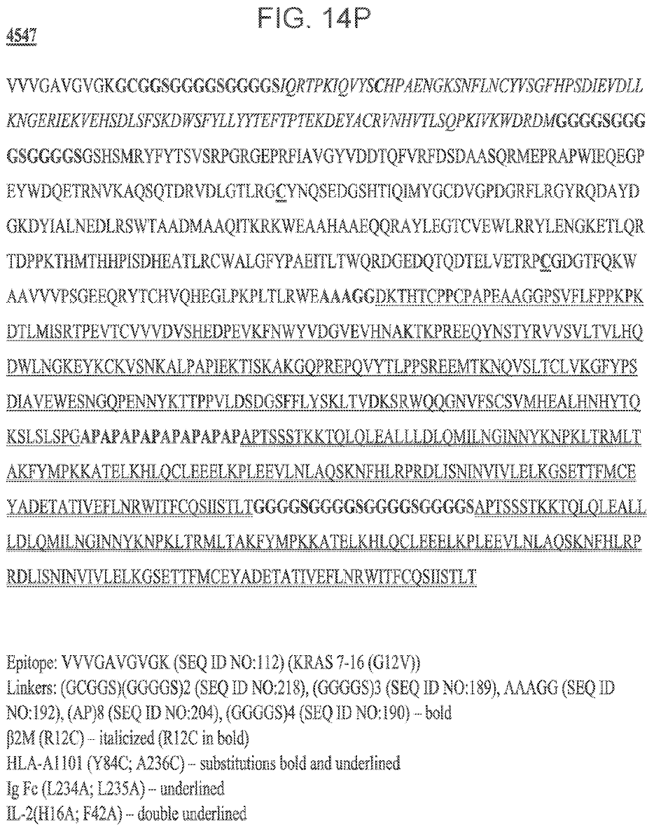

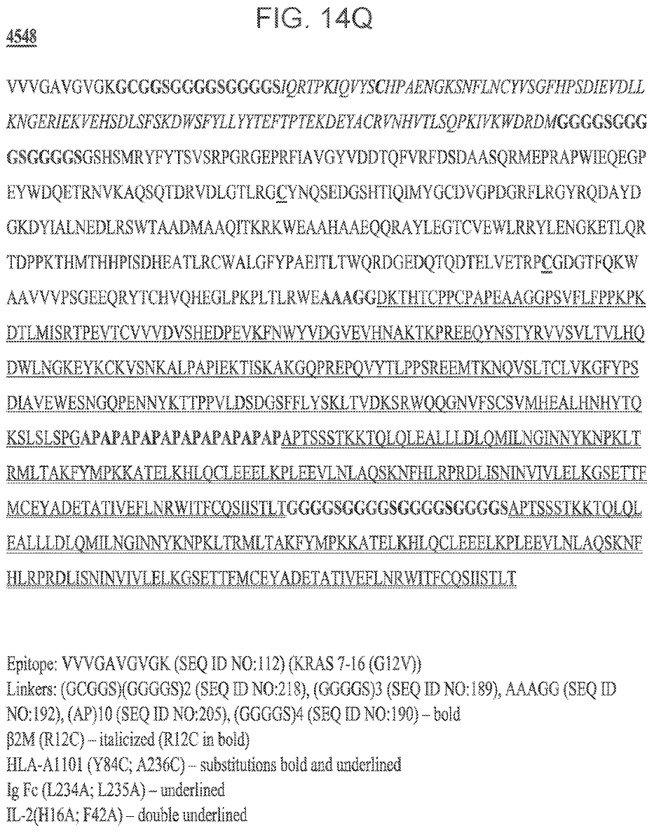

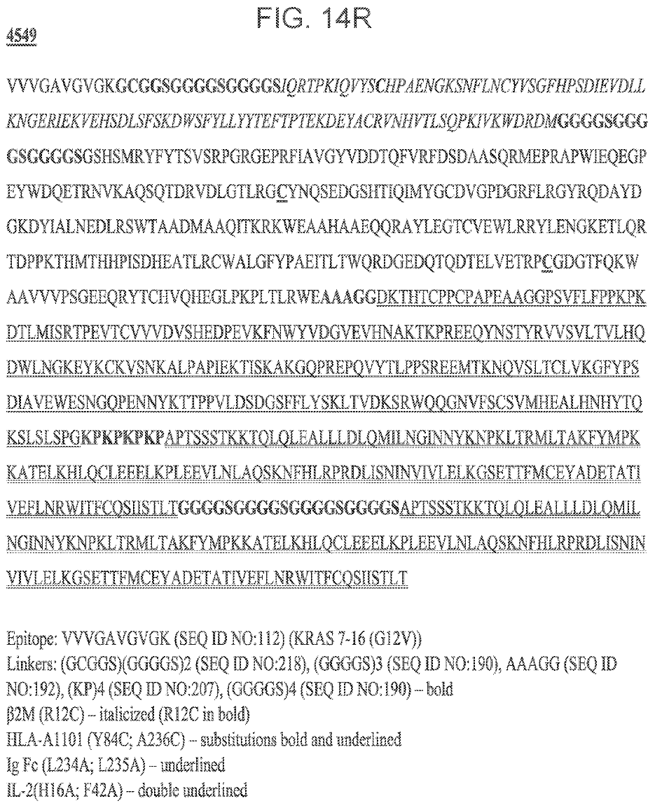

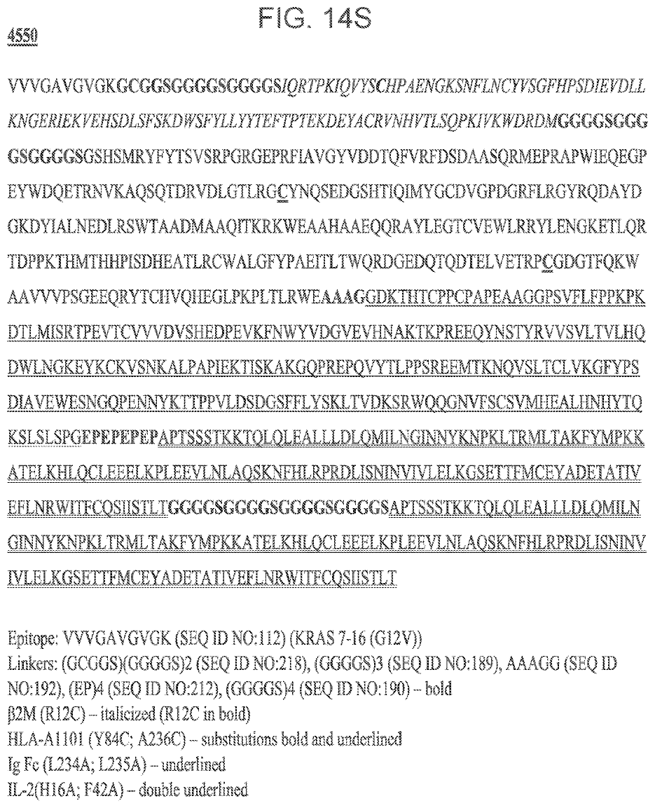

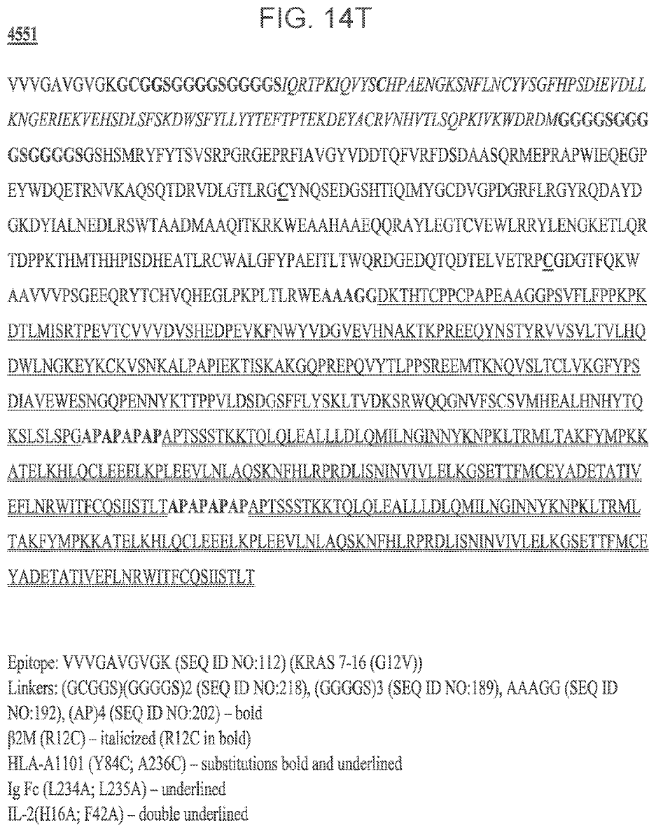

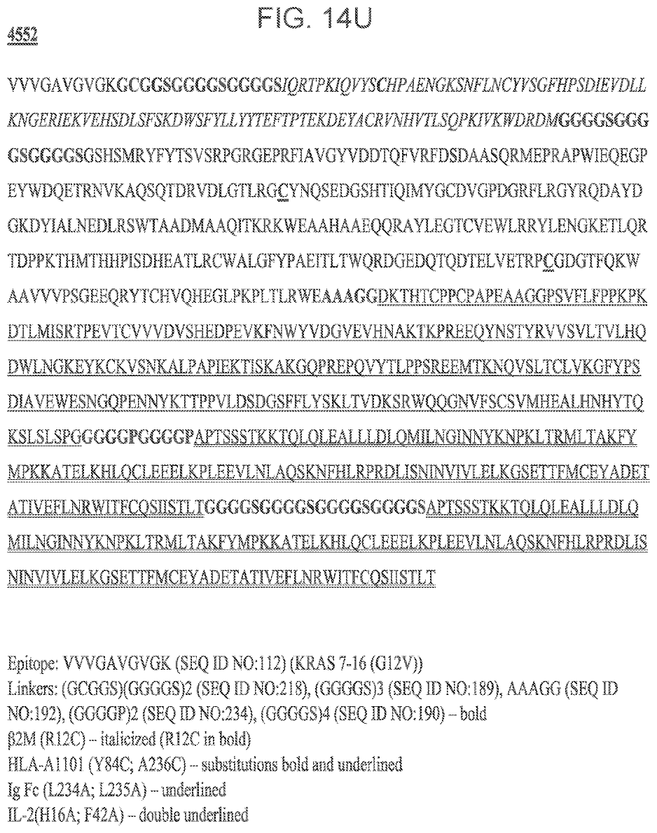

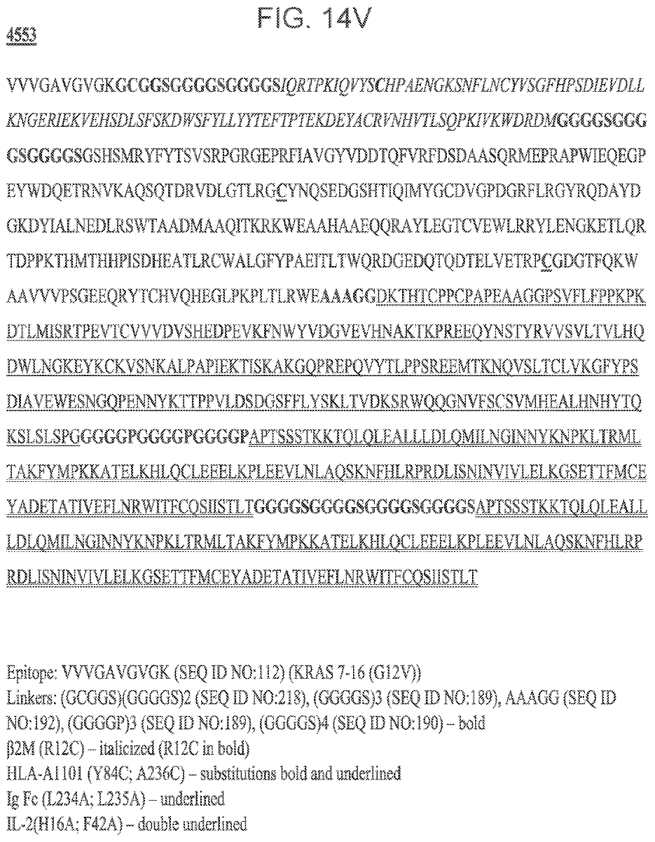

As one non-limiting example, a TMP can comprise the amino acid sequence depicted in FIG. 14A (TMP “4074”). As one non-limiting example, a TMP can comprise the amino acid sequence depicted in FIG. 14B (TMP “4390”). As one non-limiting example, a TMP can comprise the amino acid sequence depicted in FIG. 14C (TMP “4391”). As one non-limiting example, a TMP can comprise the amino acid sequence depicted in FIG. 14D (TMP “4467”). As one non-limiting example, a TMP can comprise the amino acid sequence depicted in FIG. 14E (TMP “4468”). As one non-limiting example, a TMP can comprise the amino acid sequence depicted in FIG. 14F (TMP “4469”). As one non-limiting example, a TMP can comprise the amino acid sequence depicted in FIG. 14G (TMP “4470”). As one non-limiting example, a TMP can comprise the amino acid sequence depicted in FIG. 14H (TMP “4471”). As one non-limiting example, a TMP can comprise the amino acid sequence depicted in FIG. 14I (TMP “4472”). As one non-limiting example, a TMP can comprise the amino acid sequence depicted in FIG. 14J (TMP “4473”). As one non-limiting example, a TMP can comprise the amino acid sequence depicted in FIG. 14K (TMP “4478”. As one non-limiting example, a TMP can comprise the amino acid sequence depicted in FIG. 14L (TMP “4514”). As one non-limiting example, a TMP can comprise the amino acid sequence depicted in FIG. 14M (TMP “4515”). As one non-limiting example, a TMP can comprise the amino acid sequence depicted in FIG. 14N (TMP “4545”). As one non-limiting example, a TMP can comprise the amino acid sequence depicted in FIG. 14O (TMP “4546”). As one non-limiting example, a TMP can comprise the amino acid sequence depicted in FIG. 14P (TMP “4547”). As one non-limiting example, a TMP can comprise the amino acid sequence depicted in FIG. 14Q (TMP “4548”). As one non-limiting example, a TMP can comprise the amino acid sequence depicted in FIG. 14R (TMP “4549”). As one non-limiting example, a TMP can comprise the amino acid sequence depicted in FIG. 14S (TMP “4550”). As one non-limiting example, a TMP can comprise the amino acid sequence depicted in FIG. 14T (TMP “4551”). As one non-limiting example, a TMP can comprise the amino acid sequence depicted in FIG. 14U (TMP “4552”). As one non-limiting example, a TMP can comprise the amino acid sequence depicted in FIG. 14V (TMP “4553”).

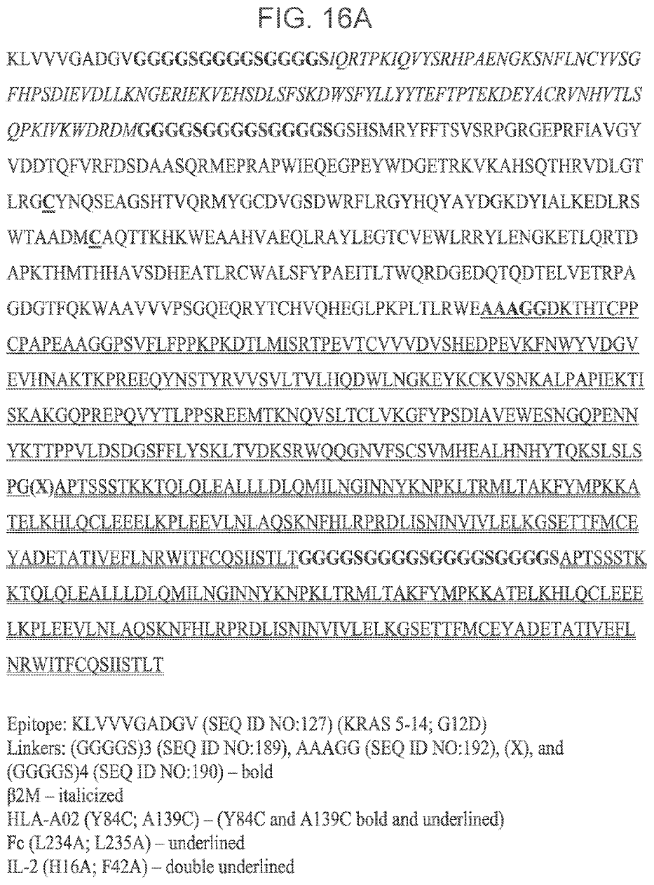

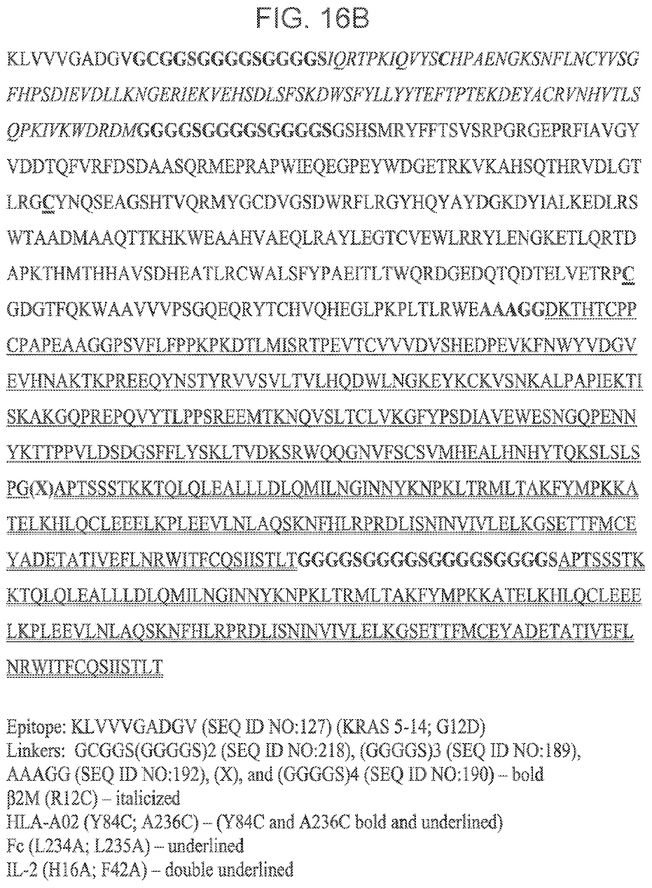

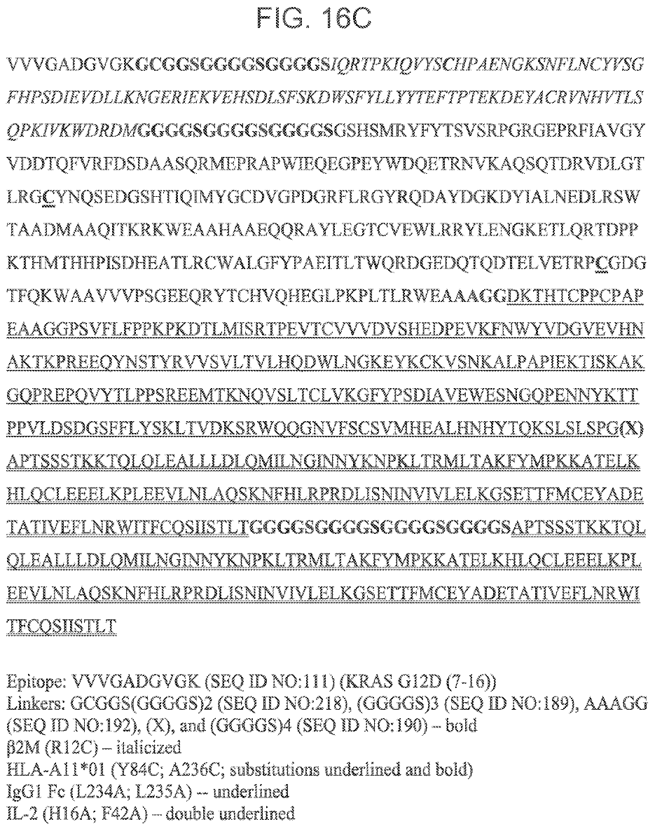

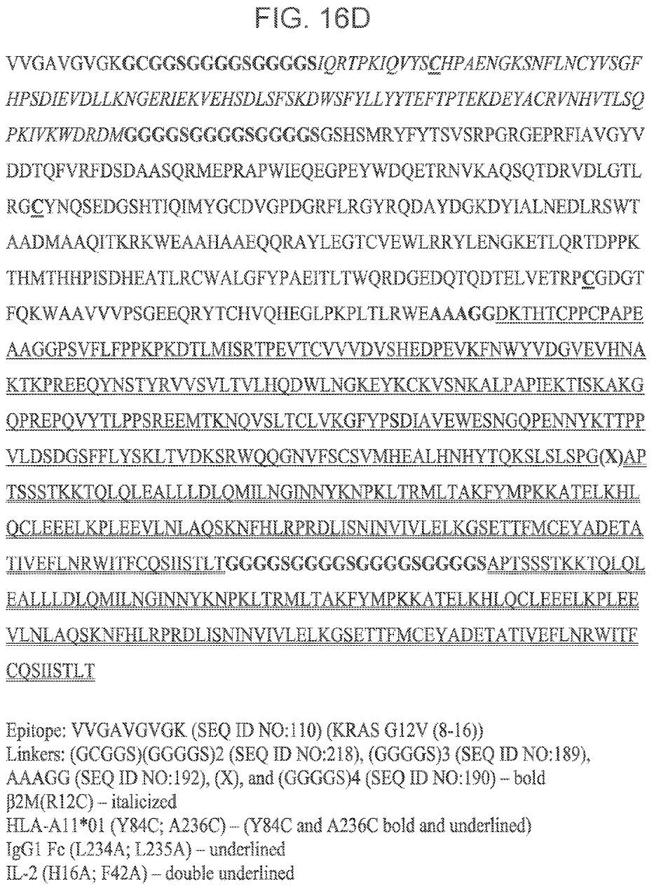

Other non-limiting examples of TMPs are provided in FIG. 16A-16D, where “(X)” is a rigid peptide linker. For example, (X) is a rigid peptide linker comprising an amino acid sequence selected from EAAAK (SEQ ID NO:194), A(EAAAK)n (SEQ ID NO:244). A(EAAAK)nA (SEQ ID NO:245), (AP)n (SEQ ID NO:246), (EP)n (SEQ ID NO:247), and (KP)n (SEQ ID NO:248), where n is an integer from 1 to 10 (e.g., n is 1, 2, 3, 4, 5, 6, 7, 8, 9, or 10). As one example, a TMP can have the amino acid sequence depicted in FIG. 16A, where (X) is (AP)n, where n is 2-8. As another example, a TMP can have the amino acid sequence depicted in FIG. 16A, where (X) is (EP)n), where n is 2-8. As another example, a TMP can have the amino acid sequence depicted in FIG. 16A, where (X) is (KP)n, where n is 2-8. As another example, a TMP can have the amino acid sequence depicted in FIG. 16A, where (X) is a peptide comprising EAAAK (SEQ ID NO:194); e.g., where (X) is A(EAAAK)nA (SEQ ID NO:245), where n is 1 or 2. As another example, a TMP can have the amino acid sequence depicted in FIG. 16B, where (X) is (EP)n), where n is 2-8. As another example, a TMP can have the amino acid sequence depicted in FIG. 16B, where (X) is (KP)n, where n is 2-8. As another example, a TMP can have the amino acid sequence depicted in FIG. 16B, where (X) is a peptide comprising EAAAK (SEQ ID NO:194); e.g., where (X) is A(EAAAK)nA (SEQ ID NO:245), where n is 1 or 2. As another example, a TMP can have the amino acid sequence depicted in FIG. 16C, where (X) is (EP)n), where n is 2-8. As another example, a TMP can have the amino acid sequence depicted in FIG. 16C, where (X) is (KP)n, where n is 2-8. As another example, a TMP can have the amino acid sequence depicted in FIG. 16C, where (X) is a peptide comprising EAAAK (SEQ ID NO:194); e.g., where (X) is A(EAAAK)nA (SEQ ID NO:245), where n is 1 or 2. As another example, a TMP can have the amino acid sequence depicted in FIG. 16D, where (X) is (EP)n), where n is 2-8. As another example, a TMP can have the amino acid sequence depicted in FIG. 16D, where (X) is (KP)n, where n is 2-8. As another example, a TMP can have the amino acid sequence depicted in FIG. 16D, where (X) is a peptide comprising EAAAK (SEQ ID NO:194); e.g., where (X) is A(EAAAK)nA (SEQ ID NO:245), where n is 1 or 2. In any of the exemplary TMPs depicted in FIG. 16A-16D, the KRAS peptide can be substituted with a different KRAS peptide; suitable KRAS peptides are described herein. In any of the exemplary TMPs depicted in FIG. 16A-16D, the peptide linker between the two copies of the variant IL-2 polypeptide can be substituted with a rigid peptide linker.

### Nucleic Acids, Recombinant Expression Vectors, and Modified Host Cells

The present disclosure provides a nucleic acid comprising a nucleotide sequence encoding a TMP of the present disclosure. In some cases, the nucleotide sequence encoding the TMP is operably linked to one or more transcriptional control elements. In some cases, the transcriptional control element is a promoter that is functional in a eukaryotic cell. In some cases, the nucleic acid is present in a recombinant expression vector.

The present disclosure thus provides recombinant expression vectors comprising nucleic acids encoding a TMP. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors are well known to persons skilled in the art. See, e.g., published PCT application WO2020132138A1 and WO2019/051091, the disclosures of which as they pertain to such expression vectors are expressly incorporated herein by reference, including specifically paragraphs [00515]-[00520] of WO2020132138A1 and paragraphs [00401]-[00406] of WO2019/051091.

The present disclosure further provides a genetically modified host cell, where the host cell is genetically modified with a nucleic acid or expression vector as described above.

Suitable host cells include eukaryotic cells, such as yeast cells, insect cells, and mammalian cells. In some cases, the host cell is a cell of a mammalian cell line. Suitable mammalian cell lines include human cell lines. non-human primate cell lines, rodent (e.g., mouse, rat) cell lines, and the like. Suitable mammalian cell lines include, but are not limited to. HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2M.

In some cases, the host cell is a mammalian cell that has been genetically modified such that it does not synthesize endogenous MHC class I heavy chain. In some cases, the host cell is a mammalian cell that has been genetically modified such that it does not synthesize endogenous MHC β2M and such that it does not synthesize endogenous MHC class I heavy chain.

### Methods of Generating a T-Cell Modulatory Polypeptide

A TMP of the present disclosure can be generated by culturing a genetically modified host cell of the present disclosure in a suitable culture medium in vitro, where such culturing results in production of the TMP. For example, a mammalian host cell (e.g., a CHO cell) can be genetically modified with a recombinant expression vector comprising a nucleotide sequence encoding a TMP of the present disclosure; and the genetically modified mammalian host cell can be cultured in vitro in a suitable culture medium, such that the genetically modified mammalian host cell produces the TMP. The TMP can be isolated. e.g., from the culture medium in which the genetically modified mammalian host cell is cultured and/or from a cell lysate of the genetically modified mammalian host cell. The TMP can be isolated using any of a variety of well-established methods. Where the TMP comprises an Ig Fc polypeptide at its C terminus, intracellular processing may remove a C-terminal Lys residue from the C-terminus of the Ig Fc polypeptide; see, e.g., van den Bremer et al. (2015) mAbs 7:4; and Sissolak et al. (2019) J. Industrial Microbiol. & Biotechnol. 46:1167. And as noted above, two TMPs that each comprise an Ig Fc polypeptide (e.g., an IgG1 Fc) may spontaneously form a homodimer of the two TMPS, wherein the individual TMPs are joined by one or more disulfide bonds between their respective Ig Fc portions.

### Compositions

The present disclosure provides compositions, including pharmaceutical compositions, comprising a TMP or dimerized TMP as disclosed herein. The present disclosure provides compositions, including pharmaceutical compositions, comprising a nucleic acid or a recombinant expression vector.

Compositions Comparing a TMP or Dimerized TMP

A composition can comprise, in addition to a TMP or dimerized TMP, one or more pharmaceutically acceptable excipients such as carriers, diluents, buffers, salts, solubilizing agents, surfactants, stabilizers, or other additives, that may, e.g., aid in the manufacturing process, protect, support or enhance stability, bioavailability and/or patient acceptability. Pharmaceutically acceptable excipients are well known to persons of skill in the art.

Where a TMP or dimerized TMP is administered as an injectable (e.g. subcutaneously, intraperitoneally, intramuscularly, and/or intravenously) directly into a tissue, a formulation can be provided as a ready-to-use dosage form that may be directly injected or infused into the patient or admixed with a saline solution for infusion, or possibly as a non-aqueous form (e.g., a reconstitutable storage-stable powder) or aqueous form. such as liquid composed of pharmaceutically acceptable carriers and excipients. Formulations may also be provided so as to enhance serum half-life of the TMP following administration. For example, the TMP or dimerized TMP may be provided in a liposome formulation, prepared as a colloid, or other conventional techniques for extending serum half-life. The preparations may also be provided in controlled release or slow-release forms.

The concentration of a TMP or dimerized TMP in a liquid composition formulation can vary widely (e.g., from less than about 0.1%. usually at or at least about 2% to as much as 20% to 50% or more by weight). Included within this range is a concentration of from about 5 to about 15 mg/mL, from about 8 to about 12 mg/mL, from about 9 to about 11 mg/mL, including about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL and about 15 mg/mL. The concentration may depend on numerous factors, including the stability of the TMP in the liquid composition.

In some cases, a TMP or dimerized TMP is present in a liquid composition. In some cases, a composition comprises: a) a TMP or dimerized TMP; and b) saline (e.g., 0.9% NaCl). In some cases, the composition is sterile and suitable for administration to a human subject.

### Compositions Comprising a Nucleic Acid or a Recombinant Expression Vector

The present disclosure provides pharmaceutical compositions. comprising a nucleic acid (e.g., DNA, RNA or mRNA) or a recombinant expression vector encoding a TMP. Published PCT applications WO2020132138A1 and WO2019/051091 disclose how to prepare such compositions. See, e.g., paragraphs [00537]-[00546] of WO2020132138A1 and paragraphs [00423]-[00432] of WO2019/051091, the disclosures of which are expressly incorporated herein by reference.

### Methods of Modulating T Cell Activity

The present disclosure provides a method of selectively modulating the activity of an epitope-specific T cell (e.g., a T cell specific for a KRAS epitope, such as a KRAS peptide comprising a cancer-associated mutation), the method comprising contacting the T cell with a TMP or dimerized TMP, where contacting the T cell with a TMP or dimerized TMP selectively modulates the activity of the epitope-specific T cell. In some cases, the contacting occurs in vitro. In some cases, the contacting occurs in vivo.

Where a TMP or dimerized TMP includes a MOD that is an activating polypeptide, contacting the T cell with the TMP or dimerized TMP activates the epitope-specific T cell, increasing the cytotoxic activity of the T cell toward a cancer cell expressing the KRAS epitope and/or increasing the number of the epitope-specific T cells.

In some cases, a TMP or dimerized TMP, when administered to an individual in need thereof, induces both an epitope-specific T cell response and an epitope non-specific T cell response. In other words, in some cases, a TMP, when administered to an individual in need thereof (i) induces an epitope-specific T cell response by modulating the activity of a first T cell that displays both a TCR specific for the KRAS epitope present in the TMP and a co-MOD that binds to the MOD present in the TMP; and (ii) induces an epitope non-specific T cell response by modulating the activity of a second T cell that displays a TCR specific for an epitope other than the KRAS epitope present in the TMP, and a co-MOD that binds to the MOD present in the TMP. The ratio of the epitope-specific T cell response to the epitope-non-specific T cell response is at least 2:1, at least 5:1, at least 10:1, at least 15:1, at least 20:1, at least 25:1, at least 50:1, or at least 100-1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100-1, or more than 100:1. This ratio is determined by measuring the increase in the number of T cells specific for the target KRAS epitope versus the increase in the number of T cells that are not specific for that target epitope. For example, conventional flow cytometry methods may be employed. “Modulating the activity” of a T cell can include one or more of the following when an activating MOD such as a variant IL-2 is present: i) activating a cytotoxic (e.g., CD8⁺) T cell; ii) inducing cytotoxic activity of a cytotoxic (e.g., CD8⁺ T cell; iii) inducing production and release of a cytotoxin (e.g., a perforin; a granzyme; a granulysin) by a cytotoxic (e.g., CD8⁺) T cell; iv) inducing proliferation of a cytotoxic (e.g., CD8⁺) T cell.

As discussed above, in some cases, a TMP or dimerized TMP, when administered to an individual in need thereof, induces a proliferation of epitope-specific T cells. The increase in the percentage of epitope-specific T cells can be measured by conventional flow cytometry methods (see, e.g., Example 2 below). Thus, e.g., the percent of total CD8+ T cells that are specific for the KRAS epitope may be increased following contact with the TMP by at least about 10%, at least about 15%, at least about 20%, at least about 25%, at least about 50%, at least about 75%, at least about 2-fold, at least about 2.5-fold, at least about 3-fold, at least about 4-fold, or higher than 4-fold.

The present disclosure provides a method of delivering a MOD selectively to target T cell, the method comprising contacting a mixed population of T cells with a TMP or dimerized TMP, where the mixed population of T cells comprises the target T cell and non-target T cells, where the target T cell is specific for the KRAS epitope present within the TMP or dimerized TMP (e.g., where the target T cell is specific for the KRAS epitope present within the TMP or dimerized TMP), and where the contacting step delivers the one or more MODs present within the TMP or dimerized TMP to the target T cell. In some cases, the population of T cells is in vitro. In some cases, the population of T cells is in vivo in an individual. In some cases, the method comprises administering the TMP or dimerized TMP to the individual. In some case, the target T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure also provides a method of detecting, in a mixed population of T cells obtained from an individual, the presence of a target T cell that binds an epitope of interest (e.g., a cancer epitope; a KRAS peptide comprising a cancer-associated mutation), the method comprising: a) contacting in vitro the mixed population of T cells with a TMP or dimerized TMP, wherein the TMP or dimerized TMP comprises the KRAS epitope of interest; and b) detecting activation and/or proliferation of T cells in response to said contacting, wherein activated and/or proliferated T cells indicates the presence of the target T cell.

### Treatment Methods

The present disclosure provides a method of treatment of an individual, the method comprising administering to the individual an amount of a TMP or dimerized TMP, or one or more nucleic acids encoding the TMP, effective to treat the individual. Also provided is a TMP or dimerized TMP for use in a method of treatment of the human or non-human animal body. In some cases, a treatment method comprises administering to an individual in need thereof one or more recombinant expression vectors comprising nucleotide sequences encoding a TMP. In some cases, a treatment method comprises administering to an individual in need thereof one or more nucleic acid molecules (e.g., mRNA molecules) comprising nucleotide sequences encoding a TMP or dimerized TMP. In some cases, a treatment method comprises administering to an individual in need thereof a TMP or dimerized TMP. Conditions that can be treated include, e.g., cancer, such as a cancer that expresses a KRAS polypeptide, e.g., a mutant KRAS polypeptide, as described above. Thus, the present disclosure provides a treatment method comprising administering to an individual in need thereof an effective amount of a TMP or dimerized TMP.

A TMP or dimerized TMP can be administered to an individual in need thereof to treat a cancer in the individual, where the cancer expresses the KRAS peptide present in the TMP or dimerized TMP. For example, the cancer can be one in which the cancer cells express or over-express KRAS. e.g., a mutated form of KRAS, as described above. The present disclosure provides a method of treating cancer in an individual, the method comprising administering to the individual an effective amount of a TMP or dimerized TMP, or one or more nucleic acids (e.g., expression vectors; mRNA; etc.) comprising nucleotide sequences encoding the IMP or dimerized TMP, where the TMP or dimerized TMP comprises a T-cell epitope that is a KRAS epitope, and where the TMP or dimerized TMP comprises a stimulatory MOD. In some cases, an “effective amount” of a TMP or dimerized TMP is an amount that, when administered in one or more doses to an individual in need thereof, either as a monotherapy or as part of a combination therapy (e.g., as part of a combination therapy with an immune checkpoint inhibitor), as discussed below. reduces the overall tumor burden in the individual, i.e., the amount of cancer in the body, or alternatively, causes the total tumor burden in the patient to remain relatively stable for a sufficient period of time for the patient to have a confirmed “stable disease” as determined by standard RECIST criteria. See, e.g., Aykan and Özatli (2020) World J. Clin. Oncol. 11:53.

In some cases, an effective amount of a TMP or dimerized TMP is an amount that, when administered in one or more doses to an individual in need thereof, either as a monotherapy or as part of a combination therapy, e.g., with an immune checkpoint inhibitor, as discussed below, causes the tumor size to be reduced by a sufficient amount, and for a sufficient period of time, for the patient to have a confirmed “partial response” as determined by standard RECIST criteria.

In some cases, an effective amount of a TMP or dimerized TMP is an amount that, when administered in one or more doses to an individual in need thereof, either as a monotherapy or as part of a combination therapy, e.g., with an immune checkpoint inhibitor, causes the tumor size to be reduced by a sufficient amount, and for a sufficient period of time, for the patient to have a confirmed “complete response” as determined by standard RECIST criteria.

In some cases, an effective amount of a TMP or dimerized TMP is an amount that, when administered in one or more doses to an individual in need thereof, either as a monotherapy or as part of a combination therapy, e.g., with an immune checkpoint inhibitor, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the TMP or dimerized TMP, or in the absence of administration with the TMP or dimerized TMP.

In some cases, an effective amount of a TMP or dimerized TMP is an amount that, when administered in one or more doses to an individual in need thereof, either as a monotherapy or as part of a combination therapy, e.g., with an immune checkpoint inhibitor, reduces the number of cancer cells in the individual, including to substantially undetectable levels.

In some cases, an effective amount of a TMP or dimerized TMP is an amount that, when administered in one or more doses to an individual in need thereof (an individual having a tumor), reduces the tumor volume in the individual. For example, in some cases, an effective amount of a TMP or dimerized TMP is an amount that, when administered in one or more doses to an individual in need thereof (an individual having a tumor), either as a monotherapy or as part of a combination therapy with an immune checkpoint inhibitor. reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the TMP or dimerized TMP, or in the absence of administration with the TMP or dimerized TMP. Tumor volume is determined using the formula (length×width×width)/2, where length represents the largest tumor diameter and width represents the perpendicular tumor diameter. In some cases, an effective amount of a TMP or dimerized TMP is an amount that, when administered in one or more doses to an individual in need thereof, either as a monotherapy or as part of a combination therapy. e.g., with an immune checkpoint inhibitor, increases the overall survival time of the individual. For example, in some cases, an effective amount of a TMP or dimerized TMP is an amount that, when administered in one or more doses to an individual in need thereof, either as a monotherapy or as part of a combination therapy, e.g., with an immune checkpoint inhibitor, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years. compared to the expected survival time of the individual in the absence of administration with the TMP or dimerized TMP.

In some cases, an effective amount of a TMP or dimerized TMP is an amount that, when administered in one or more doses to an individual in need thereof, either as a monotherapy or as part of a combination therapy, e.g., with an immune checkpoint inhibitor, reduces the level of circulating tumor DNA (“ctDNA”) in the patient by at least 10%, at least 15%, at least 20%, at least 25%, at least 30%, at least 40%, at least 50%, at least 60%, at least 70%, at least 80%, at least 90%, or at least 95%, compared to the ctDNA levels in the individual before administration of the TMP or dimerized TMP, or in the absence of administration with the TMP or dimerized TMP. The level of ctDNA can be determined using any known method, and the same method should be used with subsequent samples to determine whether there has been a reduction and the percentage of such reduction, if any.

Cancers that can be treated with a method of this disclosure include cancers in which the cancer cells express a mutated form of KRAS. Examples include adenocarcinomas and hematological malignancies. Examples of cancers that can be treated with a method of this disclosure include multiple myeloma: B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach cancer; urothelial cancer; cervical cancer; and ovarian endometrial cancer.

As noted above, in some cases, in carrying out a subject treatment method, a TMP or dimerized TMP is administered to an individual in need thereof. In other instances, in carrying out a subject treatment method, one or more nucleic acids comprising nucleotide sequences (e.g., DNA or mRNA) encoding a TMP or dimerized TMP is/are administering to an individual in need thereof. Thus, in other instances, one or more nucleic acids, e.g., one or more recombinant expression vectors, is/are administered to an individual in need thereof.

### Dosages

A suitable dosage of a TMP or dimerized TMP can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered. sex of the patient, time, and route of administration. general health, and other drugs being administered concurrently. A TMP or dimerized TMP may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. If the regimen is a continuous infusion, it can also be in the range of 1 μg to 10 mg per kilogram of body weight per minute. A TMP or dimerized TMP can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight. from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight. Exemplary amounts of TMP or dimerized TMP include from 1 mg/kg body weight to 5 mg/kg body weight, from 5 mg/kg body weight to 10 mg/kg body weight, from about 1 mg/kg body weight to about 5 mg/kg body weight, and from about 5 mg/kg body weight to about 10 mg/kg body weight.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMP or dimerized TMP is administered in maintenance doses, ranging from about 1 mg/kg body weight to about 5 mg/kg body weight, from about 5 mg/kg body weight to about 10 mg/kg body weight, from about 10 mg/kg body weight to about 15 mg/kg body weight. from about 15 mg/kg body weight to about 20 mg/kg body weight, or amounts exceeding 20 mg/kg of body weight.

Those of skill will readily appreciate that dose levels can vary as a function of the specific TMP or dimerized TMP, the severity of the symptoms and the susceptibility of the subject to side effects. Preferred dosages for a given compound are readily determinable by those of skill in the art by a variety of means.

In some cases, multiple doses of a TMP or dimerized TMP, a nucleic acid, or a recombinant expression vector are administered. The frequency of administration of a TMP or dimerized TMP, a nucleic acid, or a recombinant expression vector can vary depending on any of a variety of factors, e.g., severity of the symptoms, etc. For example, in some cases, a TMP or dimerized TMP, a nucleic acid, or a recombinant expression vector is administered once per month, twice per month, three times per month, every other week (qow), once per week (qw), once every two weeks, once every three weeks, once every four weeks, twice per week (biw), three times per week (tiw), four times per week, five times per week, six times per week, every other day (qod), daily (qd), twice a day (qid), or three times a day (tid). Where the TMP or dimerized TMP is administer intravenously, administration once every week, once every two weeks, once every three weeks or once every four weeks or once every month may be commonly employed at the beginning of treatment.

The duration of administration of a TMP or dimerized TMP, a nucleic acid, or a recombinant expression vector. e.g., the period of time over which a TMP or dimerized TMP, a nucleic acid, or a recombinant expression vector is administered, can vary, depending on any of a variety of factors, e.g., patient response, etc. For example, a TMP or dimerized TMP, a nucleic acid, or a recombinant expression vector can be administered over a period of time ranging from about one day to about one week. from about two weeks to about four weeks, from about one month to about two months. from about two months to about four months, from about four months to about six months, from about six months to about eight months, from about eight months to about 1 year. from about 1 year to about 2 years, or from about 2 years to about 4 years, or more.

### Routes of Administration

An active agent (a TMP or dimerized TMP, a nucleic acid, or a recombinant expression vector) is administered to an individual using any available method and route suitable for drug delivery, including in vivo and in vitro methods, as well as systemic and localized routes of administration.

A TIP or dimerized TMP of this disclosure typically will be delivered via intravenous administration, but other conventional and pharmaceutically acceptable routes of administration may be used, including intratumoral, peritumoral, intramuscular, intralymphatic, intratracheal, intracranial, subcutaneous, intradermal, topical application, intraarterial, rectal, nasal, oral, and other enteral and parenteral routes of administration. Routes of administration may be combined, if desired, or adjusted depending upon the TMP or dimerized TMP and/or the desired effect. A TMP or dimerized TMP, or a nucleic acid or recombinant expression vector, can be administered in a single dose or in multiple doses.

### Combination Therapy

A TMP or dimerized TMP can be administered to an individual in need thereof in combination with one or more additional therapeutic agents or therapeutic treatment. A suitable dosage of the TMP or dimerized TMP will be the same as the dosage amount for monotherapy with the TMP (described above) or may be less or more than the monotherapy dose. Suitable additional therapeutic agents include. e.g.: i) an immune checkpoint inhibitor; ii) a cancer chemotherapeutic agent; iii) an agent that inhibits a cancer-associated mutated form of KRAS; and iv) one or more additional TMPs or dimerized TMPs. Suitable additional therapeutic treatments include. e.g., radiation, surgery (e.g., surgical resection of a tumor), and the like.

In some cases, the method comprises administering to an individual in need thereof: a) a first composition comprising a TMP or dimerized TMP; and b) a second composition comprising an immune checkpoint inhibitor. In some cases, the method comprises administering to an individual in need thereof: a) a first composition comprising a TMP or dimerized TMP; and h) a second composition comprising an agent that inhibits a cancer-associated mutated form of KRAS such as KRAS (G12C or G12V). In some cases, the method comprises administering to an individual in need thereof: a) a first composition comprising a TMP or dimerized TMP; and b) a second composition comprising a second TMP or dimerized TMP.

A TMP or dimerized TMP can be administered to an individual in need thereof at the same time, or at different times, as the one or more additional therapeutic agent is administered.

Thus. for example, a treatment method can comprise co-administration of a TMP or dimerized TMP and at least one additional therapeutic agent. By “co-administration” is meant that both a TMP or dimerized TMP and at least one additional therapeutic agent are administered to an individual, although not necessarily at the same time, in order to achieve a therapeutic effect that is the result of having administered both the TMP or dimerized TMP and the at least one additional therapeutic agent. The administration of the TMP or dimerized TMP and the at least one additional therapeutic agent can be substantially simultaneous. e.g., the TMP or dimerized TMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a TMP or dimerized TMP is administered to an individual who is undergoing treatment with, or who has undergone treatment with, the at least one additional therapeutic agent. The administration of the TMP or dimerized TMP and the at least one additional therapeutic agent can occur at different times and/or at different frequencies.

As another example, a treatment method can comprise co-administration of a TMP or dimerized TMP and an immune checkpoint inhibitor such as an antibody specific for an immune checkpoint. By “co-administration” is meant that both a TMP or dimerized TMP and an antibody specific for an immune checkpoint are administered to an individual, although not necessarily at the same time, in order to achieve a therapeutic effect that is the result of having administered both the TMP or dimerized TIP and the immune checkpoint inhibitor. The administration of the TMP or dimerized TMP and the antibody specific for an immune checkpoint can be substantially simultaneous, e.g., the TMP or dimerized TMP can be administered to an individual within about 1 minute to about 24 hours (e.g., within about 1 minute, within about 5 minutes. within about 15 minutes. within about 30 minutes. within about 1 hour, within about 2 hours, within about 4 hours, within about 8 hours, within about 12 hours, or within about 24 hours) of administration of the antibody specific for an immune checkpoint. Alternatively, the TMP or dimerized TMP and immune checkpoint inhibitor can be administered on different schedules, including different days and different weeks, and different frequencies. In some cases, a TMP or dimerized TMP is administered to an individual who is undergoing treatment with, or who has undergone treatment with, an antibody specific for an immune checkpoint.

Exemplary immune checkpoint inhibitors include inhibitors that target immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1 BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122, and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA.

In some cases, the immune checkpoint inhibitor is an antibody specific for an immune checkpoint. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.). MGA271 (Macrogenics). BMS-986016 (Bristol-Meyers Squibb), lirilumab (Bristol-Myers Squibb). urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab. pembrolizumab (also known as MK-3475), pidilizumab, SHR-1210, PDR001, and AMP-224. In some cases, the anti-PD-1 monoclonal antibody is nivolumab. pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, sec, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MED14736, MPDL3280A (also known as RG7446), KN035, or MSB0010718C. In some embodiments, the anti-PD-L1 monoclonal antibody is MPDL3280A (atezolizumab) or MED14736 (durvalumab). For durvalumab, see, e.g., WO 2011/066389. For atezolizumab, see, e.g., U.S. Pat. No. 8,217,149.

Among such checkpoint inhibitors, antibodies to PD-1, PD-L1 and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. The TMPs and dimerized TMPs of this disclosure also may be co-administered with combinations of checkpoint inhibitors, e.g., a combination of (i) an antibody to PD-1 or PD-L1, and (ii) an antibody to CTLA-4.

In some cases, the at least one additional therapeutic agent is an agent that selectively inhibits a mutant form of KRAS such as KRAS (G12C), KRAS (K117A), and the like. Examples of agents that selectively inhibit a mutant form of KRAS include ARS-1620; AMG510; KRA-533; and MRTX849.

In some cases, the at least one additional therapeutic agent comprises one or more additional TMPs or dimerized TMPs. In some cases, the method comprises administering to an individual in need thereof: a) a first composition comprising a first TMP or dimerized TMP, where the first TMP or dimerized TMP is a TMP or dimerized TMP; and b) a second composition comprising a second TMP or dimerized TMP, where the second TMP or dimerized TMP is a TMP or dimerized TMP that is different from the first TMP or dimerized TMP, e.g., comprising a different KRAS epitope and/or one or more different MODs. In addition, or alternatively, the one or more additional TMP or dimerized TMPs can comprise an epitope that is a cancer-associated peptide other than a KRAS peptide epitope.

### Subjects Suitable for Treatment

Subjects suitable for treatment with a method of this disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment include individuals having a cancer in which the cancer cells express a mutated form of KRAS, where the mutated form of KRAS is a cancer-associated mutated form. Subjects suitable for treatment with a method include individuals having a cancer such as multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach cancer; urothelial cancer; cervical cancer; and ovarian endometrial cancer.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

### Detection Methods

A TMP or dimerized TMP of the present disclosure is useful for diagnostic applications and therapeutic applications. As discussed below, when used for diagnostic applications, a TMP or dimerized TMP of the present disclosure also can comprise a detectable label so that binding of the TMP or dimerized TMP to a target T cell is detected by detecting the detectable label. When used for such diagnostic applications, a TMP or dimerized TMP may not include one or more MODs, and in such cases the MOD-less TMP or dimerized TMP is referred to as an antigen presenting polypeptide (“APP”), in this case one that presents a KRAS epitope. Like TMPs, APPs also can be dimerized. The discussion below relating to detection methods using TMPs and dimerized TMPs thus is intended to apply equally to the use of APPs and dimerized APPs. The construct referred to as “4515” in FIG. 14M is an example of such an APP.

The present disclosure thus provides a method of detecting the presence and/or activation of an antigen-specific T-cell. The methods comprise contacting a T cell with a TMP/APP or dimerized TMP/APP of the present disclosure; and detecting binding of the TMP/APP or dimerized TMP/APP to the T cell, and/or activation of the T cell. The present disclosure provides a method of detecting an antigen-specific T cell, the method comprising contacting a T cell with a TMP/APP or dimerized TMP/APP of the present disclosure, wherein binding of the TMP/APP or dimerized TMP/APP to the T cell indicates that the T cell is specific for the KRAS epitope present in the TMP/APP or dimerized TMP/APP, that is, the T cell comprises a T cell receptor that is specific for the KRAS epitope present in the TMP/APP or dimerized TMP/APP.

In some cases, the TMP/APP or dimerized TMP/APP comprises a detectable label. Suitable detectable labels include, but are not limited to, a radioisotope, a fluorescent polypeptide, or an enzyme that generates a fluorescent product, and an enzyme that generates a colored product. Where the TMP/APP or dimerized TMP/APP comprises a detectable label, binding of the TMP/APP or dimerized TMP/APP to the T cell is detected by detecting the detectable label.

In some cases, a TMP/APP or dimerized TMP/APP comprises a detectable label suitable for use in in vivo imaging, e.g., suitable for use in positron emission tomography (PET), single photon emission tomography (SPECT), near infrared (NIR) optical imaging, x-ray imaging. computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7 tris(carboxymethyl)-10-(2′-hydroxypropyl)-1,4,7,10-tetraazacycl ododecane)), iron chelates, magnesium chelates, manganese chelates, copper chelates. chromium chelates, iodine-based materials, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁴Cu, ⁶⁷Cu, ⁷⁵Br, ⁷⁷Br, ⁹⁹mTc, ¹⁴C, ¹³N, ¹⁵O, ³²P, ³³P, and ¹⁸F. In some cases, the detectable label is a positron-emitting isotope such as ¹¹C, ¹³N, ¹⁵O, ¹⁸F, ⁶⁴Cu, ⁶⁸Ga, ⁷⁸Br, ⁸²Rb, ⁸⁶Y, ⁹⁰Y, ²²Na, ²⁶Al, ⁴⁰K, ⁸³Sr, ⁸⁹Zr, or ¹²⁴I. In some cases, the detectable label is ⁶⁴Cu. See. e.g., Woodham, Andrew et al., In vivo detection of antigen-specific CD8+ T cells by immuno-positron emission tomography, Nature Methods Articles (2020) https://doi.org/10.1038/s41592-020-0934-5.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein. Phycobiliprotcins and Phycobiliprotein conjugates including B-Phycocrythrin. R-Phycocrythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of a TMP/APP or dimerized TMP/APP to a T cell is detected using a detectably labeled antibody specific for the TMP/APP or dimerized TMP/APP. An antibody specific for the TMP/APP or dimerized TMP/APP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### HLA/Peptide Binding Assays

Whether a given peptide (e.g., a KRAS peptide that comprises a KRAS epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays. ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A single-chain T-cell modulatory polypeptide (TMP) comprising:
i) a KRAS peptide comprising a KRAS epitope expressed on a cancer cell, wherein the KRAS peptide has a length of from about 4 amino acids to about 25 amino acids;
ii) first major histocompatibility complex (MHC) polypeptide, wherein the first MHC polypeptide is a β2-microglobulin (β2M) polypeptide;
iii) a second MHC polypeptide, wherein the second MHC polypeptide is an MHC class I heavy chain polypeptide;
iv) one or more immunomodulatory polypeptides; and
v) an immunoglobulin (Ig) Fc polypeptide.
wherein the TMP comprises:
   (a) a rigid peptide linker, optionally wherein the TMP comprises a rigid peptide linker between the Ig Fc polypeptide and one of the one or more immunomodulatory polypeptides; and/or
   (b) a short flexible peptide linker comprising from 2-14 amino acids, optionally wherein the TMP comprises a short flexible peptide linker between the Ig Fc polypeptide and one of the one or more immunomodulatory polypeptides.
optionally wherein the TMP comprises one or more additional peptide linkers.

Aspect 2. A TMP of aspect 1, wherein the Ig Fc polypeptide comprises an amino acid sequence having at least 95% amino acid sequence identity to the amino acid sequence depicted in any one of FIG. 2A-2M.

Aspect 3. A TMP according to aspect 1 or aspect 2, wherein the Ig Fc polypeptide substantially does not induce cell lysis, optionally wherein the Ig Fc is an IgG1 Fc polypeptide that comprises one or more amino acid substitutions selected from the group consisting of N297A, L234A, L235A, L234F, L235E, and P331S.

Aspect 4. A TMP according to any one of aspects 1-3, wherein the TMP comprises one or more intrachain disulfide bonds.

Aspect 5. A TMP of aspect 4, wherein the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide.

Aspect 6. A TMP of aspect 4 or aspect 5, wherein the β2-microglobulin polypeptide is joined to the KRAS peptide by a first linker comprising a Cys, and wherein a disulfide bond links a Cys present in the first linker with a Cys present in the MHC heavy chain polypeptide, optionally wherein the first linker comprises the sequence CGGGS(GGGGS)n (SEQ ID NO:220) or GCGGS(GGGGS)n (SEQ ID NO:217), where n is an integer from 1-10, e.g., 2 or 3, and a disulfide bond links the Cys in the linker with a Cys substituted for Tyr-84 of the MHC heavy chain polypeptide.

Aspect 7. A TMP of any one of aspects 1-6, wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, and an HLA-A*2401 polypeptide.

Aspect 8. A TMP of any one of aspects 1-6, wherein the MHC heavy chain polypeptide comprises an amino acid sequence having at least 95% amino acid sequence identity to an HLA-E polypeptide or an HLA-G polypeptide.

Aspect 9. A TMP of any one of aspects 1-8, wherein the at least one immunomodulatory polypeptide is a wild-type or variant of an activating immunomodulatory polypeptide selected from the group consisting of a IL-2, a 4-1BBL, CD80, CD86, or combinations thereof, optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

Aspect 10. A TMP of aspect 9, wherein the at least one immunomodulatory polypeptide is a variant of IL-2 that exhibits decreased binding affinity for IL-2Rα and IL-2Rβ, optionally wherein the variant IL-2 polypeptide comprises i) an H16A substitution and an F42A substitution; or ii) an H16T substitution and an F42A substitution.

Aspect 11. A TMP of aspect 9 or 10, wherein the TMP comprises two copies of the immunomodulatory polypeptide.

Aspect 12. A TMP of aspect 11, wherein the two copies of the immunomodulatory polypeptide are in tandem, optionally wherein a peptide linker is interposed between the two copies.

Aspect 13. A TMP of any one of aspects 1-12, wherein the KRAS peptide has a length of from 8 amino acids to 12 amino acids.

Aspect 14. A TMP of any of aspects 1-13, wherein the KRAS peptide comprises a sequence selected from the group consisting of:
A) VVGADGVGK (SEQ ID NO:108), VVGACGVGK (SEQ ID NO:109), VVGAVGVGK (SEQ ID NO:110), VVVGADGVGK (SEQ ID NO:111), VVVGAVGVGK (SEQ ID NO:112), VVVGACGVGK (SEQ ID NO: 113), VTGADGVGK (SEQ ID NO: 114), VTGAVGVGK (SEQ ID NO:115), VTGACGVGK (SEQ ID NO:116), VTVGADGVGK (SEQ ID NO:117), VTVGAVGVGK (SEQ ID NO:118), and VTVGACGVGK (SEQ ID NO:119); and wherein the KRAS peptide has a length of 9 amino acids or 10 amino acids, or a length of at least 9 amino acids or 10 amino acids;
B) VVVGAGDVGK (SEQ ID NO:120); VVGAGDVGK (SEQ ID NO:121); VVVGARGVGK (SEQ ID NO:122); and VVGARGVGK (SEQ ID NO:123); and wherein the KRAS peptide has a length of 9 amino acids or 10 amino acids, or a length of 9 amino acids or 10 amino acids;
C) LVVVGADGV (SEQ ID NO:124), LVVVGAVGV (SEQ ID NO:125), LVVVGACGV (SEQ ID NO:126), KLVVVGADGV (SEQ ID NO:127), KLVVVGAVGV (SEQ ID NO:128), KLVVVGACGV (SEQ ID NO:129), LLVVGADGV (SEQ ID NO:130), LLVVGAVGV (SEQ ID NO:131), LLVVGACGV (SEQ ID NO:132), FLVVVGADGV (SEQ ID NO:133), FLVVVGAVGV (SEQ ID NO:134), and FLVVVGACGV (SEQ ID NO:135); and wherein the KRAS peptide has a length of 9 amino acids or 10 amino acids, or a length of 9 amino acids or 10 amino acids;
D) KLVVVGAGDV (SEQ ID NO:136); and KLVVVGARGV (SEQ ID NO:137); wherein the KRAS peptide has a length of 9 amino acids or 10 amino acids;
E) GAGDVGKSAL (SEQ ID NO:138); AGDVGKSAL (SEQ ID NO:139); DVGKSALTI (SEQ ID NO:140); GAVGVGKSAL (SEQ ID NO:141); AVGVGKSAL (SEQ ID NO:142); YKLVVVGAV (SEQ ID NO:143); ARGVGKSAL (SEQ ID NO:144); GARGVGKSAL (SEQ ID NO:145); EYKLVVVGAR (SEQ ID NO:146); RGVGKSALTI (SEQ ID NO:147); LVVVGARGV (SEQ ID NO:148); GADGVGKSAL (SEQ ID NO:149); ACGVGKSAL (SEQ ID NO:150); and GACGVGKSAL (SEQ ID NO:151); wherein the KRAS peptide has a length of 9 amino acids or 10 amino acids; and
F) VVGAVGVGK (SEQ ID NO:110), VVVGAVGVGK (SEQ ID NO:112), VGAVGVGKS (SEQ ID NO:152), VGAVGVGKSA (SEQ ID NO:153.), AVGVGKSAL (SEQ ID NO:142), AVGVGKSALT (SEQ ID NO:154), GAVGVGKSAL (SEQ ID NO: 141), GAVGVGKSA (SEQ ID NO:155), LVVVGAVGVG (SEQ ID NO:156), LVVVGAVGV (SEQ ID NO:125), KLVVVGAVGV (SEQ ID NO:128), and KLVVVGAVG (SEQ ID NO:157); where the KRAS peptide has a length of 9 amino acids or 10 amino acids.

Aspect 15. A T-cell modulatory polypeptide of any of aspects 1-13, wherein:
A) the KRAS peptide is KLVVVGADGV (SEQ ID NO:127) and the MHC heavy chain polypeptide comprises an amino acid sequence having at least 95% amino acid sequence identity to an HLA-A*0201 polypeptide; or
B) the KRAS peptide is VVVGADGVGK (SEQ ID NO:111) or VVGAVGVGK (SEQ ID NO:110), and the MHC heavy chain polypeptide comprises an amino acid sequence having at least 95% amino acid sequence identity to an HLA-A11*01 polypeptide.

Aspect 16. A TMP of any one of aspects 1-15. comprising in order from N-terminus to C-terminus:
i) the KRAS peptide;
ii) optionally a peptide linker;
iii) the β2M polypeptide;
iv) optionally a peptide linker;
v) the MHC class I heavy chain polypeptide;
vi) optionally a peptide linker;
vii) an Ig Fc polypeptide;
viii) a rigid peptide linker or a short flexible peptide linker; and
ix) the one or more immunomodulatory polypeptides, optionally wherein when two or more immunomodulatory polypeptides are present, a peptide linker is interposed between each of the two or more immunomodulatory polypeptide.

Aspect 17. A TMP of aspect 16, wherein the TMP comprises, in order from N-terminus to C-terminus:
i) the KRAS peptide;
ii) a flexible peptide linker;
iii) the β2M polypeptide;
iv) a flexible peptide linker;
v) the MHC class I heavy chain polypeptide;
vi) a rigid peptide linker or a short flexible peptide linker;
vii) a first copy of the at least one immunomodulatory polypeptide;
viii) a flexible peptide linker; and
ix) a second copy of the at least one immunomodulatory polypeptide, and
optionally wherein the peptide linker between the KRAS peptide and the β2M polypeptide comprises a cysteine.

Aspect 18. A TMP of aspect 16, wherein the TMP comprises, in order from N-terminus to C-terminus:
i) the KRAS peptide;
ii) a flexible peptide linker;
iii) the β2M polypeptide;
iv) a flexible peptide linker;
v) the MHC class I heavy chain polypeptide;
vi) a rigid peptide linker or a short flexible peptide linker;
vii) a first copy of the at least one immunomodulatory polypeptide;
viii) a rigid peptide linker or a short flexible peptide linker; and
ix) a second copy of the at least one immunomodulatory polypeptide, and
optionally wherein the peptide linker between the KRAS peptide and the β2M polypeptide comprises a cysteine.

Aspect 19. A TMP of aspect 17 or aspect 18, wherein the first and the second copies of the at least one immunomodulatory polypeptide are a variant of IL-2 that exhibits decreased binding affinity for IL-2Rα and IL-2Rβ, optionally wherein the variant IL-2 polypeptide comprises i) an H16A substitution and an F42A substitution; or ii) an H16T substitution and an F42A substitution.

Aspect 20. A TMP of any one of aspects 1-15, comprising in order from N-terminus to C-terminus:
i) the KRAS peptide;
ii) optionally a peptide linker;
iii) the β2M polypeptide;
iv) optionally a peptide linker;
v) the MHC class I heavy chain polypeptide;
vi) optionally a peptide linker;
vii) the one or more immunomodulatory polypeptides;
viii) the rigid peptide linker or a short flexible peptide linker; and
ix) an Ig Fc polypeptide, and
optionally wherein the peptide linker between the KRAS peptide and the β2M polypeptide comprises a cysteine.

Aspect 21. A TMP of any one of aspects 1-20, wherein each of the optional one or more additional peptide linkers, if present, is independently selected from the group consisting of:
i) CGGGS(GGGGS)n (SEQ ID NO:220), where n is an integer from 1-10;
ii) GCGGS(GGGGS)n (SEQ ID NO:217), where n is an integer from 1-10;
iii) (GGGGS)n(GCGGS) (SEQ ID NO:228), where n is an integer from 1-10;
iv) (GGGGS)n (SEQ ID NO:235), where n is an integer from 1-10; and
v) AAAGG (SEQ ID NO:192).

Aspect 22. A TMP of any one of aspects 1-20, wherein each of the optional one or more additional peptide linkers, if present, is independently selected from the group consisting of:
i) CGGGS(GGGGS)n (SEQ ID NO:220), where n is 2, 3, or 4;
ii) GCGGS(GGGGS)n (SEQ ID NO:217), where n is 2, 3, or 4;
iii) (GGGGS)n(GCGGS) (SEQ ID NO:228), where n is 2, 3, or 4; and
iv) (GGGGS)n (SEQ ID NO:180), where n is 2, 3, 4, or 7; and
v) AAAGG (SEQ ID NO:192).

Aspect 23. A TMP of any one of aspects 1-22, wherein the TMP comprises at least one rigid peptide linker, and wherein each rigid peptide linker is independently selected from the group consisting of:
i) (AP)n (SEQ ID NO:246), where n is an integer from 1-10;
ii) (EP)n (SEQ ID NO:247), where n is an integer from 1-10;
iii) (KP)n (SEQ ID NO:248), where n is an integer from 1-10; and
iv) a peptide comprising EAAAK (SEQ ID NO:194).

Aspect 24. A TMP of any one of aspects 1-22, wherein the TMP comprises at least one rigid peptide linker, and wherein each rigid peptide linker is independently selected from the group consisting of:
i) (AP)n (SEQ ID NO:203), where n is 2-6;
ii) (EP)n (SEQ ID NO:213), where n is 2-6;
iii) (KP)n (SEQ ID NO:208), where n is 2-6; and
iv) A(EAAAK)nA (SEQ ID NO:200), where n is 1 or 2.

Aspect 25. A TMP of aspect 1, wherein:
a) the β2M polypeptide is joined to the KRAS peptide by a first linker that comprises the sequence CGGGS(GGGGS)n (SEQ ID NO:220) or GCGGS(GGGGS)n (SEQ ID NO:217), where n is an integer from 1-10. optionally wherein n is 2 or 3;
b) the MHC heavy chain polypeptide comprises a Cys at residue 84 and a Cys at residue 236;
c) the β2M polypeptide comprises a Cys at residue 12;
d) the Cys at amino acid residue 12 of the β2M polypeptide is disulfide bonded to a Cys at amino acid residue 236 of the MHC heavy chain polypeptide;
e) a disulfide bond links the Cys in the linker between the KRAS peptide and the β2M polypeptide with a Cys substituted for Tyr84 of the MHC heavy chain polypeptide;
f) the β2M polypeptide is connected to the MHC heavy chain polypeptide by a (GGGGS)n linker (SEQ ID NO:235), where n is 1, 2, 3, 4, 5, 6, 7, 8, 9, or 10, optionally wherein n is 3 or 7;
g) the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, an HLA-A*2401 polypeptide, an HLA-E polypeptide, and an HLA-G polypeptide;
h) the TMP comprises two immunomodulatory polypeptides that are the same, are in tandem, and comprise a variant of IL-2 that comprises i) an H16A substitution and an F42A substitution; or ii) an H16T substitution and an F42A substitution; and
i) the TMP comprises at least one rigid peptide linker, and wherein the at least one rigid peptide linker is selected from the group consisting of: i) (AP)n (SEQ ID NO:246), where n is an integer from 1-10; ii) (EP)n (SEQ ID NO:247), where n is an integer from 1-10, optionally wherein n is 2-6; iii) (KP)n (SEQ ID NO:248), where n is an integer from 1-10; and iv) a peptide comprising EAAAK (SEQ ID NO:194).

Aspect 26. A TMP of aspect 25, wherein:
a) the β2M polypeptide is joined to the KRAS peptide by a first linker that comprises the sequence GCGGS(GGGGS)n (SEQ ID NO:218), where n is 2;
f) the β2M polypeptide is connected to the MHC heavy chain polypeptide by a (GGGGS)n (SEQ ID NO:253) linker, where n is 3 or 7;
g) the MHC heavy chain polypeptide comprises an amino acid sequence having at least 95% amino acid sequence identity to an HLA-A*1101 polypeptide;
h) the TMP comprises two immunomodulatory polypeptides that are the same, are in tandem, and comprise a variant of IL-2 that comprises an H16A substitution and an F42A substitution, and wherein the two immunomodulatory polypeptides are connected by a peptide linker; and
i) the at least one rigid peptide linker comprises the amino acid sequence (AP)n, where n is an integer from 1-10, optionally wherein n is 2-6.

Aspect 27. A TMP of aspect 1, wherein the TMP comprises the amino acid sequence set forth in any one of FIGS. 14A-14L and 14N-14V.

Aspect 28. A TMP of aspect 1, wherein the TMP comprises the amino acid sequence set forth in any one of FIG. 16A-16D, wherein (X) is selected from the group consisting of:
i) (AP)n (SEQ ID NO:246), where n is an integer from 1-10;
ii) (EP)n (SEQ ID NO:247), where n is an integer from 1-10;
iii) (KP)n (SEQ ID NO:248), where n is an integer from 1-10; and
iv) a peptide comprising EAAAK (SEQ ID NO:194).

Aspect 29. A TMP of aspect 28, wherein (X) is selected from the group consisting of:
i) (AP)n, where n is 2-6 (SEQ ID NO:203);
ii) (EP)n, where n is 2-6 (SEQ ID NO:213);
iii) (KP)n, where n is 2-6 (SEQ ID NO:208); and
iv) A(EAAAK)nA (SEQ ID NO:200), where n is 1 or 2.

Aspect 30. A TMP of any one of aspects 1-22, wherein the TMP comprises at least one short flexible peptide linker, and wherein each short flexible peptide linker is independently selected from the group consisting of flexible peptide linkers comprising a number of amino acids selected from the group consisting of 2-4 aas, 2-5 aas, 3-6 aas, 4-8 aas, 5-10 aas and 10-14 aas.

Aspect 31. A TMP of any aspect 30, wherein the short flexible peptide linker comprises a number of amino acids selected from the group consisting of 2-4 aas, 2-5 aas, 3-6 aas, and 4-8 aas, and optionally wherein the short flexible peptide linker is GGS.

Aspect 32. A TMP of aspect 30 or 31, wherein:
a) the β2M polypeptide is joined to the KRAS peptide by a first linker that comprises the sequence CGGGS(GGGGS)n (SEQ ID NO:220) or GCGGS(GGGGS)n (SEQ ID NO:217), where n is an integer from 1-10. optionally wherein n is 2 or 3;
b) the MHC heavy chain polypeptide comprises a Cys at residue 84 and a Cys at residue 236;
c) the β2M polypeptide comprises a Cys at residue 12;
d) the Cys at amino acid residue 12 of the β2M polypeptide is disulfide bonded to a Cys at amino acid residue 236 of the MHC heavy chain polypeptide;
e) a disulfide bond links the Cys in the linker between the KRAS peptide and the β2M polypeptide with a Cys substituted for Tyr84 of the MHC heavy chain polypeptide;
f) the β2M polypeptide is connected to the MHC heavy chain polypeptide by a (GGGGS)n (SEQ ID NO:235) linker, where n is 1, 2, 3, 4, 5, 6, 7, 8, 9, or 10. optionally wherein n is 3 or 7;
g) the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, an HLA-A*2401 polypeptide, an HLA-E polypeptide, and an HLA-G polypeptide;
h) the TMP comprises two immunomodulatory polypeptides that are the same, are in tandem, and comprise a variant of IL-2 that comprises i) an H16A substitution and an F42A substitution; or ii) an H16T substitution and an F42A substitution.

Aspect 33. A TMP of aspect 32, wherein:
a) the β2M polypeptide is joined to the KRAS peptide by a first linker that comprises the sequence GCGGS(GGGGS)n (SEQ ID NO:218), where n is 2;
f) the β2M polypeptide is connected to the MHC heavy chain polypeptide by a (GGGGS)n (SEQ ID NO:253) linker, where n is 3 or 7;
g) the MHC heavy chain polypeptide comprises an amino acid sequence having at least 95% amino acid sequence identity to an HLA-A*1101 polypeptide;
h) the TMP comprises two immunomodulatory polypeptides that are the same, are in tandem, and comprise a variant of IL-2 that comprises an H16A substitution and an F42A substitution, and wherein the two immunomodulatory polypeptides are connected by a peptide linker.

Aspect 34. A homodimer comprising a first and second TMP of any one of aspects 1-33, wherein the first and second TMPs are the same and are covalently bound by one or more disulfide bonds between the Ig Fc polypeptides of the first and second TMPs.

Aspect 35. A heterodimer comprising a first and second TMP of any one of aspects 1-33, wherein the first and second TMPs are not the same and are bound by one or covalent bonds.

Aspect 36. A nucleic acid comprising a nucleotide sequence encoding a TMP according to any one of aspects 1-33.

Aspect 37. A recombinant expression vector comprising the nucleic acid of aspect 36.

Aspect 38. A cell genetically modified with the nucleic acid of aspect 36 or the recombinant expression vector of aspect 37.

Aspect 39. A pharmaceutical composition comprising:
a) a TMP according to any one of aspects 1-33 or a homodimer of aspect 34 or a heterodimer according to aspect 35; and
b) a pharmaceutically acceptable excipient.

Aspect 40. A method of making a TMP according to any one of aspects 1-33 or a homodimer according to aspect 34 or a heterodimer according to aspect 35, the method comprising culturing the cell of aspect 38 in vitro in a cell culture medium.

Aspect 41. A method of selectively modulating the activity of T cell specific for a KRAS peptide epitope, the method comprising contacting the T cell with a T-cell modulatory polypeptide according to any one of aspects 1-33 or a homodimer according to aspect 34 or a heterodimer according to aspect 35, wherein said contacting selectively modulates the activity of the epitope-specific T cell.

Aspect 42. A method of treating a KRAS-associated cancer in a patient having the cancer, the method comprising administering to the patient an effective amount of a pharmaceutical composition comprising a TMP according to any one of aspects 1-33 or a homodimer according to aspect 34 or a heterodimer according to aspect 35.

Aspect 43. A method of aspect 42, further comprising co-administering one or more checkpoint inhibitors to the patient.

Aspect 44. A method of aspect 43, wherein the immune checkpoint inhibitor is an antibody specific for PD-L1, PD-1, or CTLA4, optionally wherein the immune checkpoint inhibitor is selected from the group consisting of nivolumab, tremelimumab, pembrolizumab, ipilimumab, cemiplimab, atezolizumab, avelumab, tisleizumab, durvalumab, and combinations thereof.

Aspect 45. A method of increasing the thermal stability of a T-cell modulatory polypeptide (TMP) comprising:
i) a KRAS peptide comprising a KRAS epitope expressed on a cancer cell, wherein the KRAS peptide has a length of from about 4 amino acids to about 25 amino acids;
ii) first major histocompatibility complex (MHC) polypeptide, wherein the first MHC polypeptide is a β2-microglobulin (β2M) polypeptide;
iii) a second MHC polypeptide, wherein the second MHC polypeptide is an MHC class I heavy chain polypeptide;
iv) one or more immunomodulatory polypeptides (MODs); and
v) an immunoglobulin (Ig) Fc polypeptide,
wherein, when the TMP comprises one or more Position-2 MODs, the method comprises interposing a rigid peptide linker or short flexible linker between one or more of (i) the MHC class I heavy chain polypeptide and a MOD and (ii) the Ig Fc polypeptide and a MOD, and optionally, when two or more MODs are present, between at least two of the two or more MODs, and
wherein, when the TMP comprises one or more Position 3 MODs, the method comprises interposing a rigid peptide linker or short flexible linker between the Ig Fc polypeptide and a MOD, and optionally, when two or more MODs are present, between at least two of the two or more MODs.

Aspect 46. A method according to aspect 45, wherein each rigid peptide linker is independently selected from the group consisting of:
i) (AP)n (SEQ ID NO:246), where n is an integer from 1-10;
ii) (EP)n (SEQ ID NO:247), where n is an integer from 1-10;
iii) (KP)n (SEQ ID NO:248), where n is an integer from 1-10; and
iv) a peptide comprising EAAAK (SEQ ID NO: 194).

Aspect 47. A method according to aspect 45, wherein the TMP comprises at least one rigid peptide linker, and wherein each rigid peptide linker is independently selected from the group consisting of:
i) (AP)n (SEQ ID NO:203), where n is 2-6;
ii) (EP)n (SEQ ID NO:213), where n is 2-6;
iii) (KP)n (SEQ ID NO:208), where n is 2-6; and
iv) A(EAAAK)nA (SEQ ID NO:200), where n is 1 or 2.

Aspect 48. A method according to aspect 45, wherein each short flexible peptide linker is independently selected from the group consisting of flexible peptide linkers comprising a number of amino acids selected from the group consisting of 24 aas, 2-5 aas, 3-6 aas, 4-8 aas, 5-10 aas and 10-14 aas.

Aspect 49. A method according to aspect 45, wherein each short flexible peptide linker is independently selected from the group consisting of 2-4 aas, 2-5 aas, 3-6 aas, and 4-8 aas, and optionally wherein the short flexible peptide linker is GGS.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A single-chain T-cell modulatory polypeptide (TMP) comprising:
i) a KRAS peptide comprising a KRAS epitope expressed on a cancer cell, wherein the KRAS peptide has a length of from about 7 amino acids to about 16 amino acids. optionally from 8 amino acids to 12 amino acids;
ii) first major histocompatibility complex (MHC) polypeptide, wherein the first MHC polypeptide is a β2-microglobulin (β2M) polypeptide;
iii) a second MHC polypeptide, wherein the second MHC polypeptide is an MHC class I heavy chain polypeptide;
iv) one or more immunomodulatory polypeptides; and
v) an immunoglobulin (Ig) Fc polypeptide,
wherein the TMP comprises: (a) at least one rigid peptide linker, optionally wherein the TMP comprises a rigid peptide linker between the Ig Fc polypeptide and one of the one or more immunomodulatory polypeptides; and/or (b) at least one short flexible peptide linker comprising from 2-14 amino acids, optionally wherein the TMP comprises a short flexible peptide linker between the Ig Fc polypeptide and one of the one or more immunomodulatory polypeptides, optionally wherein the TMP comprises one or more additional peptide linkers.

Aspect 2. A TMP of aspect 1, wherein the Ig Fc polypeptide comprises an amino acid sequence having at least about 95% amino acid sequence identity to the amino acid sequence depicted in any one of FIG. 2A-2M.

Aspect 3. A TMP according to aspect 2, wherein the Ig Fc polypeptide substantially does not induce cell lysis, optionally wherein the Ig Fc is an IgG1 Fc polypeptide that comprises one or more amino acid substitutions selected from the group consisting of N297A, L234A, L235A, L234F, L235E, and P331S.

Aspect 4. A TMP according to aspect 1, wherein the TMP comprises one or more intrachain disulfide bonds.

Aspect 5. A TMP of aspect 4, wherein:
(a) the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide;
(b) the β2-microglobulin polypeptide is joined to the peptide by a first linker comprising a Cys, and wherein a disulfide bond links a Cys present in the first linker with a Cys present in the MHC heavy chain polypeptide, optionally wherein the first linker comprises the sequence CGGGS(GGGGS)n (SEQ ID NO:220) or GCGGS(GGGGS)n (SEQ ID NO:217), where n is an integer from 1-10, e.g., 2 or 3, and a disulfide bond links the Cys in the linker with a Cys substituted for Tyr84 of the MHC heavy chain polypeptide; or
(c) the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide, and the β2-microglobulin polypeptide is joined to the peptide by a first linker comprising a Cys, and wherein a disulfide bond links a Cys present in the first linker with a Cys present in the MHC heavy chain polypeptide. optionally wherein the first linker comprises the sequence CGGGS(GGGGS)n (SEQ ID NO:220) or GCGGS(GGGGS)n (SEQ ID NO:217), where n is an integer from 1-10. e.g., 2 or 3, and a disulfide bond links the Cys in the linker with a Cys substituted for Tyr84 of the MHC heavy chain polypeptide.

Aspect 6. A TMP of aspect 1, wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, and an HLA-A*2401 polypeptide.

Aspect 7. A TMP of aspect 1, wherein the MHC heavy chain polypeptide comprises an amino acid sequence having at least 95% amino acid sequence identity to an HLA-E polypeptide or an HLA-G polypeptide.

Aspect 8. A TMP of aspect 1, wherein the at least one immunomodulatory polypeptide is a wild-type or variant of an activating immunomodulatory polypeptide selected from the group consisting of an IL-2 polypeptide, a 4-1BBL polypeptide, CD80 polypeptide. CD86 polypeptide, or combinations thereof, optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide,

Aspect 9. A TMP of aspect 8, wherein the at least one immunomodulatory polypeptide is a variant of IL-2 that exhibits decreased binding affinity for IL-2Rα and IL-2Rβ, optionally wherein the variant IL-2 polypeptide comprises i) an H16A substitution and an F42A substitution; or ii) an H16T substitution and an F42A substitution.

Aspect 10. A TMP of aspect 1, wherein the KRAS peptide comprises a sequence selected from the group consisting of:
A) VVGADGVGK (SEQ ID NO:108), VVGACGVGK (SEQ ID NO:109), VVGAVGVGK (SEQ ID NO:110), VVVGADGVGK (SEQ ID NO:111), VVVGAVGVGK (SEQ ID NO: 112), VVVGACGVGK (SEQ ID NO:113), VTGADGVGK (SEQ ID NO: 114), VTGAVGVGK (SEQ ID NO: 115). VTGACGVGK (SEQ ID NO: 116), VTVGADGVGK (SEQ ID NO: 117), VTVGAVGVGK (SEQ ID NO:118), and VTVGACGVGK (SEQ ID NO: 119); and wherein the KRAS peptide has a length of 9 amino acids or 10 amino acids, or a length of at least 9 amino acids or 10 amino acids;
B) VVVGAGDVGK (SEQ ID NO:120); VVGAGDVGK (SEQ ID NO:121); VVVGARGVGK (SEQ ID NO:122); and VVGARGVGK (SEQ ID NO:123); and wherein the KRAS peptide has a length of 9 amino acids or 10 amino acids, or a length of 9 amino acids or 10 amino acids;
C) LVVVGADGV (SEQ ID NO:124), LVVVGAVGV (SEQ ID NO: 125), LVVVGACGV (SEQ ID NO:126), KLVVVGADGV (SEQ ID NO:127), KLVVVGAVGV (SEQ ID NO:128), KLVVVGACGV (SEQ ID NO:129), LLVVGADGV (SEQ ID NO:130), LLVVGAVGV (SEQ ID NO:131), LLVVGACGV (SEQ ID NO:132), FLVVVGADGV (SEQ ID NO:133), FLVVVGAVGV (SEQ ID NO:134), and FLVVVGACGV (SEQ ID NO:135); and wherein the KRAS peptide has a length of 9 amino acids or 10 amino acids, or a length of 9 amino acids or 10 amino acids;
D) KLVVVGAGDV (SEQ ID NO:136); and KLVVVGARGV (SEQ ID NO:137); wherein the KRAS peptide has a length of 9 amino acids or 10 amino acids;
E) GAGDVGKSAL (SEQ ID NO:138); AGDVGKSAL (SEQ ID NO:139); DVGKSALTI (SEQ ID NO:140); GAVGVGKSAL (SEQ ID NO:141); AVGVGKSAL (SEQ ID NO:142); YKLVVVGAV (SEQ ID NO:143); ARGVGKSAL (SEQ ID NO:144); GARGVGKSAL (SEQ ID NO:145); EYKLVVVGAR (SEQ ID NO:146); RGVGKSALTI (SEQ ID NO:147); LVVVGARGV (SEQ ID NO:148); GADGVGKSAL (SEQ ID NO:149); ACGVGKSAL (SEQ ID NO:150); and GACGVGKSAL (SEQ ID NO:151); wherein the KRAS peptide has a length of 9 amino acids or 10 amino acids; and
F) VVGAVGVGK (SEQ ID NO:110), VVVGAVGVGK (SEQ ID NO:112), VGAVGVGKS (SEQ ID NO:152), VGAVGVGKSA (SEQ ID NO:153), AVGVGKSAL (SEQ ID NO:142), AVGVGKSALT (SEQ ID NO:154), GAVGVGKSAL (SEQ ID NO:141), GAVGVGKSA (SEQ ID NO:155), LVVVGAVGVG (SEQ ID NO:156), LVVVGAVGV (SEQ ID NO:125), KLVVVGAVGV (SEQ ID NO:128), and KLVVVGAVG (SEQ ID NO:157); where the KRAS peptide has a length of 9 amino acids or 10 amino acids.

Aspect 11. A T-cell modulatory polypeptide of aspect 1, wherein:
A) the KRAS peptide is KLVVVGADGV (SEQ ID NO:127) and the MHC heavy chain polypeptide comprises an amino acid sequence having at least 95% amino acid sequence identity to an HLA-A*0201 polypeptide; or
B) the KRAS peptide is VVVGADGVGK (SEQ ID NO:111) or VVGAVGVGK (SEQ ID NO:110), and the MHC heavy chain polypeptide comprises an amino acid sequence having at least 95% amino acid sequence identity to an HLA-A11*01 polypeptide.

Aspect 12. A TMP of aspect 1, comprising in order from N-terminus to C-terminus:
i) the KRAS peptide;
ii) optionally a peptide linker;
iii) the β2M polypeptide;
iv) optionally a peptide linker;
v) the MHC class I heavy chain polypeptide;
vi) optionally a peptide linker;
vii) an Ig Fc polypeptide;
viii) a rigid peptide linker or a short flexible peptide linker; and
ix) the one or more immunomodulatory polypeptides, optionally wherein when two or more immunomodulatory polypeptides are present, a peptide linker is interposed between each of the two or more immunomodulatory polypeptide; or
i) the KRAS peptide;
ii) a flexible peptide linker;
iii) the β2M polypeptide;
iv) a flexible peptide linker;
v) the MHC class I heavy chain polypeptide;
vi) a flexible peptide linker;
vii) the Ig Fc polypeptide;
viii) a rigid peptide linker or a short flexible peptide linker;
ix) a first copy of the at least one immunomodulatory polypeptide;
x) a peptide linker; and
xi) a second copy of the at least one immunomodulatory polypeptide, and
optionally wherein the peptide linker between the KRAS peptide and the β2M polypeptide comprises a cysteine; or
i) the KRAS peptide;
ii) a flexible peptide linker;
iii) the β2M polypeptide;
iv) a flexible peptide linker;
v) the MHC class I heavy chain polypeptide;
vi) a rigid peptide linker or a short flexible peptide linker;
vii) a first copy of the at least one immunomodulatory polypeptide;
viii) a rigid peptide linker or a short flexible peptide linker;
ix) a second copy of the at least one immunomodulatory polypeptide,
x) a peptide linker; and
xi) the Ig Fc polypeptide; and
optionally wherein the peptide linker between the KRAS peptide and the β2M polypeptide comprises a cysteine; or
i) the KRAS peptide;
ii) optionally a peptide linker;
iii) the β2M polypeptide;
iv) optionally a peptide linker;
v) the MHC class I heavy chain polypeptide;
vi) optionally a peptide linker;
vii) the one or more immunomodulatory polypeptides;
viii) the rigid peptide linker or a short flexible peptide linker; and
ix) an Ig Fc polypeptide, and

optionally wherein the peptide linker between the KRAS peptide and the β2M polypeptide comprises a cysteine, and optionally wherein when two or more immunomodulatory polypeptides are present, a peptide linker is interposed between each of the two or more immunomodulatory polypeptide.

Aspect 13. A TMP of aspect 1, wherein each of the optional one or more additional peptide linkers, if present, is independently selected from the group consisting of:
i) CGGGS(GGGGS)n (SEQ ID NO:220), where n is an integer from 1-10, optionally wherein n is 2, 3, or 4;
ii) CGGGS((GGGGS)n (SEQ ID NO:217), where n is an integer from 1-10, optionally wherein n is 2, 3, or 4;
iii) (GGGGS)n(GCGGS), where n is an integer from 1-10. optionally wherein n is 2, 3, or 4;
iv) (GGGGS)n where n is an integer from 1-10, optionally wherein n is 2, 3, 4, or 7; and
v) AAAGG.

Aspect 14. A TMP of aspect 1, wherein the TMP comprises at least one rigid peptide linker, and wherein each rigid peptide linker is independently selected from the group consisting of:
i) (AP)n, where n is an integer from 1-10. optionally where n is 2-6;
ii) (EP)n, where n is an integer from 1-10, optionally where n is 2-6;
iii) (KP)n, where n is an integer from 1-10. optionally where n is 2-6; and
iv) a peptide comprising EAAAK, optionally where n is 1 or 2.

Aspect 15. A TMP of aspect 1, wherein:
a) the β2M polypeptide is joined to the KRAS peptide by a first linker that comprises the sequence CGGGS(GGGGS)n (SEQ ID NO:220) or GCGGS(GGGGS)n (SEQ ID NO:217), where n is an integer from 1-10, optionally wherein n is 2 or 3;
b) the MHC heavy chain polypeptide comprises a Cys at residue 84 and a Cys at residue 236;
c) the β2M polypeptide comprises a Cys at residue 12;
d) the Cys at amino acid residue 12 of the β2M polypeptide is disulfide bonded to a Cys at amino acid residue 236 of the MHC heavy chain polypeptide;
e) a disulfide bond links the Cys in the linker between the KRAS peptide and the β2M polypeptide with a Cys substituted for Tyr84 of the MHC heavy chain polypeptide;
f) the β2M polypeptide is connected to the MHC heavy chain polypeptide by a (GGGGS)n linker, where n is 1, 2, 3, 4, 5, 6, 7, 8, 9, or 10, optionally wherein n is 3 or 7;
g) the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, an HLA-A*2401 polypeptide, an HLA-E polypeptide, and an HLA-G polypeptide;
h) the TMP comprises two immunomodulatory polypeptides that are the same, are in tandem, and comprise a variant of IL-2 that comprises i) an H16A substitution and an F42A substitution; or ii) an H16T substitution and an F42A substitution; and
i) the TMP comprises at least one rigid peptide linker, and wherein the at least one rigid peptide linker is selected from the group consisting of: i) (AP)n, where n is an integer from 1-10; ii) (EP)n, where n is an integer from 1-10, optionally wherein n is 2-6; iii) (KP)n, where n is an integer from 1-10; and iv) a peptide comprising EAAAK.

Aspect 16. A TMP of aspect 15, wherein:
a) the β2M polypeptide is joined to the KRAS peptide by a first linker that comprises the sequence GCGGS(GGGGS)n (SEQ ID NO:217), where n is 2;
f) the β2M polypeptide is connected to the MHC heavy chain polypeptide by a (GGGGS)n linker, where n is 3 or 7;
g) the MHC heavy chain polypeptide comprises an amino acid sequence having at least 95% amino acid sequence identity to an HLA-A*1101 polypeptide;
h) the TMP comprises two immunomodulatory polypeptides that are the same, are in tandem, and comprise a variant of IL-2 that comprises an H16A substitution and an F42A substitution, and wherein the two immunomodulatory polypeptides are connected by a peptide linker; and
i) the at least one rigid peptide linker comprises the amino acid sequence (AP)n, where n is an integer from 1-10, optionally wherein n is 2-6.

Aspect 17. A TMP of aspect 1, wherein the TMP comprises the amino acid sequence set forth in any one of FIGS. 14A-14L and 14N-14V.

Aspect 18. A TMP of aspect 1, wherein the TMP comprises at least one short flexible peptide linker, and wherein each short flexible peptide linker is independently selected from the group consisting of flexible peptide linkers comprising a number of amino acids selected from the group consisting of 2-4 aas, 2-5 aas, 3-6 aas, 4-8 aas, 5-10 aas and 10-14 aas.

Aspect 19. A homodimer comprising a first and second TMP of any one of aspects 1-18, wherein the first and second TMPs are the same and are covalently bound by one or more disulfide bonds between the Ig Fc polypeptides of the first and second TMPs.

Aspect 20. A heterodimer comprising a first and second TMP of any one of aspects 1-18, wherein the first and second TMPs are not the same and are bound by one or covalent bonds.

Aspect 21. A nucleic acid comprising a nucleotide sequence encoding a TMP according to any one of aspects 1-18.

Aspect 22. A recombinant expression vector comprising the nucleic acid of aspect 21.

Aspect 23. A pharmaceutical composition comprising a TMP according to any one of aspects 1-18 or a homodimer of aspect 19 or a heterodimer according to aspect 20.

Aspect 24. A method of selectively modulating the activity of T cell specific for a KRAS peptide epitope, the method comprising contacting the T cell with a T-cell modulatory polypeptide according to any one of aspects 1-18 or a homodimer according to aspect 19 or a heterodimer according to aspect 20, wherein said contacting selectively modulates the activity of the epitope-specific T cell.

Aspect 25. A method of treating a KRAS-associated cancer in a patient having the cancer, the method comprising administering to the patient an effective amount of a pharmaceutical composition comprising a TMP according to any one of aspects 1-18 or a homodimer according to aspect 19 or a heterodimer according to aspect 20.

Aspect 26. A method of aspect 25, further comprising co-administering one or more immune checkpoint inhibitors to the patient, optionally wherein the immune checkpoint inhibitor is an antibody specific for PD-L1, PD-1, TIGIT, or CTLA4.

### EXAMPLES

The following examples are put forth so as to provide those of ordinary skill in the art with a complete disclosure and description of how to make and use the present disclosure, and are not intended to limit the scope of the disclosure, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight. temperature is in degrees Celsius. and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Thermal Stability of Dimerized TMPs

Thermal stability of dimerized TMPs was assessed using an accelerated stability assay conducted at 4° C., 37° C., and at 42° C. Compositions of dimerized TMPs were kept at the indicated temperatures in a solution (phosphate-buffered saline (PBS) containing 500 mM NaCl. pH 7.4), at a concentration of 10 mg of dimerized TMP/mL solution, for a period of time of 14 days. A 1 day, 7 days, and 14 days, the percent monomer remaining in the solution was determined using size exclusion chromatography. The PBS solution was as follows: 10.14 mM sodium phosphate dibasic, 1.76 mM potassium phosphate monobasic, 2.7 mM KCl, and 0.5 M NaCl; pH 7.4.

TMPs that spontaneously form homodimers were test for stability at 4° C. 37° C., and 42° C. The amino acid sequences of the single-chain TMPs that were tested are provided in FIG. 19C and FIG. 14A-14V. The amino acid sequence of TMP 4074 is depicted in FIG. 19C. TMP 4074 comprises: i) G12V (7-16) as the peptide epitope; VVVGAVGVGK (SEQ ID NO:112)); (ii) the linker GCGGS(GGGGS)2 (SEQ ID NO:218); iii) a β2M a Cys at amino acid 12 (“R12C”); iv) a (GGGGS)3 (SEQ ID NO:189) linker; v) an HLA-A*11:01 allele heavy chain polypeptide comprising a Cys at amino acid 84 (“Y84C”) and a Cys at amino acid 236 (“A236C”); vi) an AAAGG (SEQ ID NO:192) linker; vii) an Ig Fc polypeptide comprising Ala residues at positions 14 and 15 (“L234A”; and “L235A”); viii) a (GGGGS)3 (SEQ ID NO:189) linker; ix) a first copy of a variant IL-2 polypeptide having Ala residues at amino acids 16 (“H16A”) and 42 (“F42A”); x) a (GGGGS)4 (SEQ ID NO:190) linker, and xi) a second copy of the variant IL-2 polypeptide. This TMP spontaneously formed a homodimer having disulfide bonds between the Ig Fc polypeptides of each TMP. The dimerized TMPs that were tested differ from dimerized TMP 4074 as set out in “construct features” in Table 2 below.

The data are summarized in Table 2. below. The % monomer remaining after 14 days at the indicated temperatures is provided.

TABLE 2

| Construct | 4° C. | 37° C. | 42° C. | Construct features that differ from TMP 4074 |
|---|---|---|---|---|
| 4074 | 100.1 | 46.8 | 3.1 | — |
| 4076 | 100.0 | 95.6 | 85.8 | no MOD |
| 4333 | 98.5 | 53.4 | 6.3 | (G4S)7 linker between β2M and MHC H chain |
| 4390 | 99.3 | 6.9 | 0.0 | (G4S)7 linker between β2M and MHC H chain; |
| | | | | no disulfide linkages |
| 4391 | 99.9 | 62.4 | 8.8 | (G4S)7 linker between β2M and MHC H chain |
| 4467 | 99.9 | 67.3 | 12.8 | short flexible peptide linker (GGS) between Ig Fc |
| | | | | and MOD |
| 4468 | 99.6 | 81.0 | 30.3 | only a single copy of the MOD |
| 4469 | 101.2 | 81.7 | 28.1 | only a single copy of the MOD; shortened linker |
| | | | | between Ig Fc and MOD |
| 4470 | 99.2 | 80.2 | 25.5 | (AP)4 linker between Ig Fc and MOD |
| 4471 | 99.8 | 58.9 | 4.7 | (A(EAAAK)2A) linker between Ig Fc and MOD |
| 4472 | 99.5 | 53.6 | 5.3 | A139C in MHC H chain |
| 4473 | 93.8 | 48.5 | 5.7 | A139C in MHC H chain; wild-type β2M (no R12C) |

The above data, and in particular the thermal stability data for dimerized TMPs 4470 and 4471, indicate that when the flexible (G4S)3 (SEQ ID NO:189) linker between the Ig Fc polypeptide and the variant IL-2 polypeptide in the dimerized TMP 4470 is replaced with a rigid peptide linker ((AP)4 (SEQ ID NO:202) or A(EAAAK)2A (SEQ ID NO:220)) between the Ig Fc polypeptide and the variant IL-2 polypeptide, the dimerized TMP exhibits improved stability profiles at both 37° C. and 42° C. when compared to TMP 4074. As also can be seen from the stability data for dimerized TMP 4467, shortening the flexible peptide linker between the Ig Fc polypeptide from a (GGGGS)3 (SEQ ID NO:189) linker to a GGS linker also provided improved thermal stability profiles at both 37° C. and 42° C. when compared to TMP 4074.

### Example 2: In Vitro Activity of the Dimerized TMPs

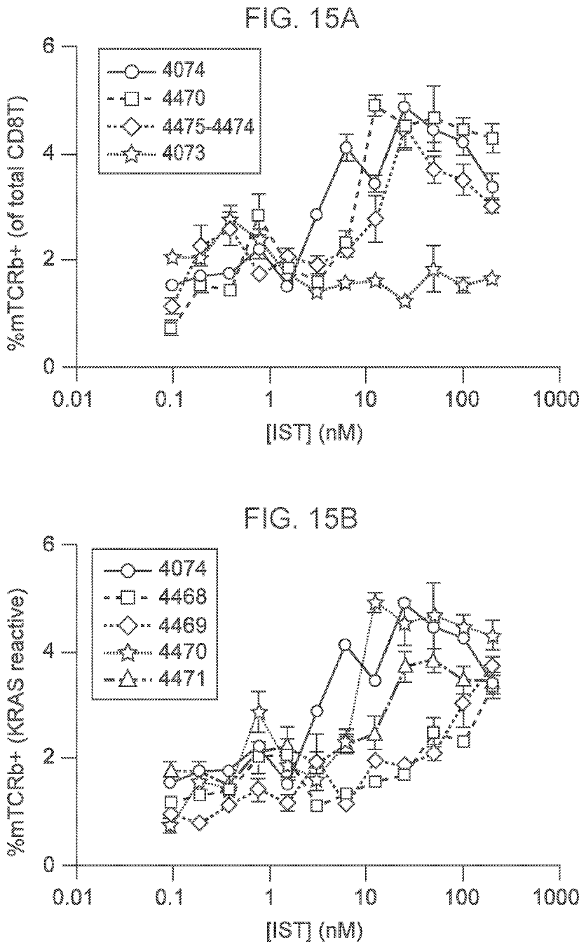

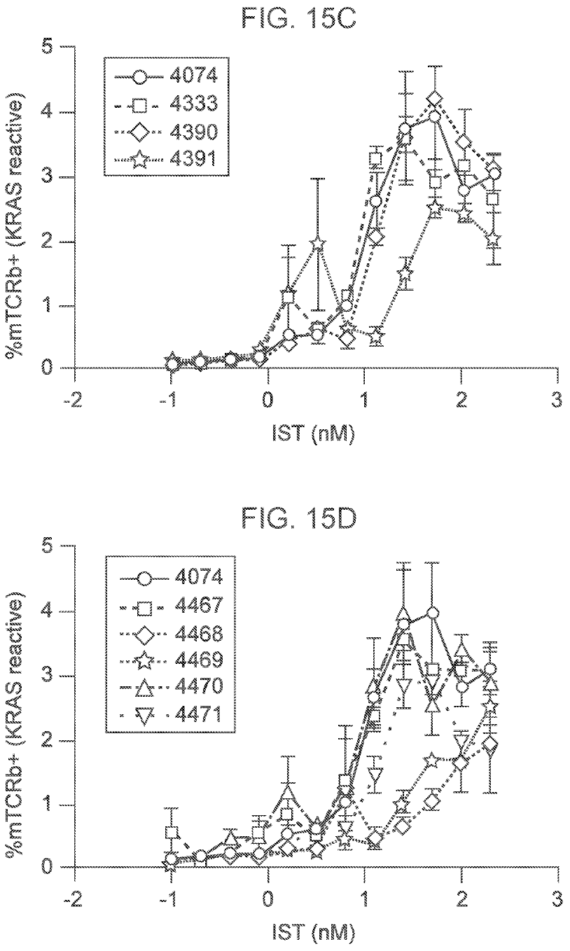

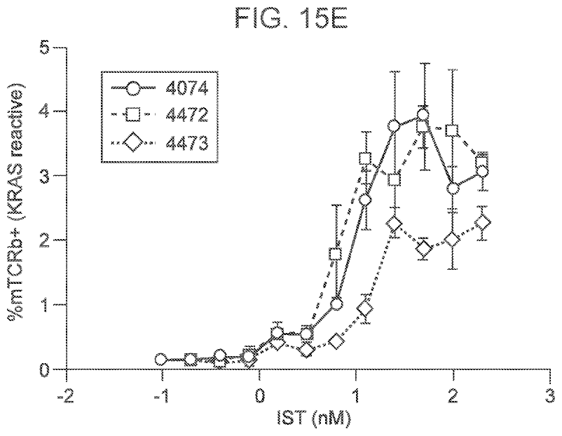

The ability of the dimerized TMPs to activate mutant KRAS G12V reactive T cells was performed using human CD8+ T cells transduced with a T cell receptor (TCR1), containing the constant region of mouse TCR (mTCR), reactive to KRAS G12V (amino acids 7-16) presented in the context of HLA-A11; these cells are referred to as “TCR-transduced CD8⁺ T cells”. A fixed number of TCR-transduced CD8⁺ T cells were mixed with autologous peripheral blood mononuclear cells (PBMC), to generate a cell mixture. The cell mixture was exposed to various concentrations of single-chain TMPs presenting KRAS G12V 7-16 and incubated at 37° C. for 9 days. Total cells were harvested, and the percentage of KRAS-reactive T cells amongst all CD8+ T cells in the cell mixture was determined by flow cytometry for the presence of mTCR+ cells. The data are depicted in FIG. 15A-15E.

The data indicate that the dimerized TMPs comprising the rigid peptide linkers, i.e., 4470 (with a rigid (AP)4 linker between the Ig Fc polypeptide and the variant IL-2 polypeptide) and 4471 (with a rigid peptide linker A(EAAAK)2A (SEQ ID NO:200) between the Ig Fc polypeptide and the variant IL-2 polypeptide) showed in vitro activity. Likewise, the dimerized construct 4467 with the shortened GGS linker between the Ig Fc polypeptide and the variant IL-2 polypeptide also showed in vitro activity.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope. All such modifications are intended to be within the scope of the claims appended hereto.

1. A single-chain T-cell modulatory polypeptide (TMP) comprising:
i) a KRAS peptide comprising a KRAS epitope expressed on a cancer cell, wherein the KRAS peptide has a length of from about 7 amino acids to about 16 amino acids, optionally from 8 amino acids to 12 amino acids;
ii) first major histocompatibility complex (MHC) polypeptide, wherein the first MHC polypeptide is a β2-microglobulin (β2M) polypeptide;
iii) a second MHC polypeptide, wherein the second MHC polypeptide is an MHC class I heavy chain polypeptide;
iv) one or more immunomodulatory polypeptides; and
v) an immunoglobulin (Ig) Fc polypeptide,
wherein the TMP comprises:
(a) at least one rigid peptide linker, optionally wherein the TMP comprises a rigid peptide linker between the Ig Fc polypeptide and one of the one or more immunomodulatory polypeptides; and/or
(b) at least one short flexible peptide linker comprising from 2-14 amino acids, optionally wherein the TMP comprises a short flexible peptide linker between the Ig Fc polypeptide and one of the one or more immunomodulatory polypeptides,
optionally wherein the TMP comprises one or more additional peptide linkers.

2. A TMP of claim 1, wherein the Ig Fc polypeptide comprises an amino acid sequence having at least about 95% amino acid sequence identity to the amino acid sequence set forth in any one of SEO ID NOs:5-17.

3. A TMP according to claim 2, wherein the Ig Fc polypeptide substantially does not induce cell lysis, optionally wherein the Ig Fc is an IgG1 Fc polypeptide that comprises one or more amino acid substitutions selected from the group consisting of N297A, L234A, L235A, L234F, L235E, and P331S, wherein N297 corresponds to N77, L234 corresponds to L14, L235 corresponds to L15, and P331 corresponds to P111 based on the amino acid numbering of the amino acid sequence set forth in SEQ ID NO:454.

4. A TMP according to claim 1, wherein the TMP comprises one or more intrachain disulfide bonds.

5. A TMP of claim 4, wherein:
(a) the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide;
(b) the β2-microglobulin polypeptide is joined to the peptide by a first linker comprising a Cys, and wherein a disulfide bond links a Cys present in the first linker with a Cys present in the MHC heavy chain polypeptide, optionally wherein the first linker comprises the sequence CGGGS(GGGGS)n (SEQ ID NO:220) or GCGGS(GGGGS)n (SEQ ID NO:217), where n is an integer from 1-10, e.g., 2 or 3, and a disulfide bond links the Cys in the linker with a Cys substituted for Tyr84 of the MHC heavy chain polypeptide; or
(c) the β2M polypeptide and the MHC heavy chain polypeptide are joined by a disulfide bond that joins a Cys residue in the β2M polypeptide and a Cys residue in the MHC heavy chain polypeptide, optionally wherein a Cys at amino acid residue 12 of the β2M polypeptide is disulfide bonded to a Cys at amino acid residue 236 of the MHC heavy chain polypeptide, and the β2-microglobulin polypeptide is joined to the peptide by a first linker comprising a Cys, and wherein a disulfide bond links a Cys present in the first linker with a Cys present in the MHC heavy chain polypeptide, optionally wherein the first linker comprises the sequence CGGGS(GGGGS)n (SEQ ID NO:220) or GCGGS(GGGGS)n (SEQ ID NO:217), where n is an integer from 1-10, e.g., 2 or 3, and a disulfide bond links the Cys in the linker with a Cys substituted for Tyr84 of the MHC heavy chain polypeptide.

6. A TMP of claim 1, wherein the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, and an HLA-A*2401 polypeptide.

7. A TMP of claim 1, wherein the MHC heavy chain polypeptide comprises an amino acid sequence having at least 95% amino acid sequence identity to an HLA-E polypeptide or an HLA-G polypeptide.

8. A TMP of claim 1, wherein the at least one immunomodulatory polypeptide is a wild-type or variant of an activating immunomodulatory polypeptide selected from the group consisting of an IL-2 polypeptide, a 4-1BBL polypeptide, CD80 polypeptide, CD86 polypeptide, or combinations thereof, optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide,

9. A TMP of claim 8, wherein the at least one immunomodulatory polypeptide is a variant of IL-2 that exhibits decreased binding affinity for IL-2Rα and IL-2Rβ, optionally wherein the variant IL-2 polypeptide comprises i) an H16A substitution and an F42A substitution; or ii) an H16T substitution and an F42A substitution.

10. A TMP of claim 1, wherein the KRAS peptide comprises a sequence selected from the group consisting of:
A) VVGADGVGK (SEQ ID NO:108), VVGACGVGK (SEQ ID NO:109), VVGAVGVGK (SEQ ID NO:110), VVVGADGVGK (SEQ ID NO:111), VVVGAVGVGK (SEQ ID NO:112), VVVGACGVGK (SEQ ID NO:113), VTGADGVGK (SEQ ID NO:114), VTGAVGVGK (SEQ ID NO:115), VTGACGVGK (SEQ ID NO:116), VTVGADGVGK (SEQ ID NO:117), VTVGAVGVGK (SEQ ID NO:118), and VTVGACGVGK (SEQ ID NO:119); and wherein the KRAS peptide has a length of 9 amino acids or 10 amino acids, or a length of at least 9 amino acids or 10 amino acids;
B) VVVGAGDVGK (SEQ ID NO:120); VVGAGDVGK (SEQ ID NO:121); VVVGARGVGK (SEQ ID NO:122); and VVGARGVGK (SEQ ID NO:123); and wherein the KRAS peptide has a length of 9 amino acids or 10 amino acids, or a length of 9 amino acids or 10 amino acids;
C) LVVVGADGV (SEQ ID NO:124), LVVVGAVGV (SEQ ID NO:125), LVVVGACGV (SEQ ID NO:126), KLVVVGADGV (SEQ ID NO:127), KLVVVGAVGV (SEQ ID NO:128), KLVVVGACGV (SEQ ID NO:129), LLVVGADGV (SEQ ID NO:130), LLVVGAVGV (SEQ ID NO:131), LLVVGACGV (SEQ ID NO:132), FLVVVGADGV (SEQ ID NO:133), FLVVVGAVGV (SEQ ID NO:134), and FLVVVGACGV (SEQ ID NO:135); and wherein the KRAS peptide has a length of 9 amino acids or 10 amino acids, or a length of 9 amino acids or 10 amino acids;
D) KLVVVGAGDV (SEQ ID NO:136); and KLVVVGARGV (SEQ ID NO:137); wherein the KRAS peptide has a length of 9 amino acids or 10 amino acids;
E) GAGDVGKSAL (SEQ ID NO:138); AGDVGKSAL (SEQ ID NO:139); DVGKSALTI (SEQ ID NO:140); GAVGVGKSAL (SEQ ID NO:141); AVGVGKSAL (SEQ ID NO:142); YKLVVVGAV (SEQ ID NO:143); ARGVGKSAL (SEQ ID NO:144); GARGVGKSAL (SEQ ID NO:145); EYKLVVVGAR (SEQ ID NO:146); RGVGKSALTI (SEQ ID NO:147); LVVVGARGV (SEQ ID NO:148); GADGVGKSAL (SEQ ID NO:149); ACGVGKSAL (SEQ ID NO:150); and GACGVGKSAL (SEQ ID NO:151); wherein the KRAS peptide has a length of 9 amino acids or 10 amino acids; and
F) VVGAVGVGK (SEQ ID NO:110), VVVGAVGVGK (SEQ ID NO:112), VGAVGVGKS (SEQ ID NO:152), VGAVGVGKSA (SEQ ID NO:153), AVGVGKSAL (SEQ ID NO:142), AVGVGKSALT (SEQ ID NO:154), GAVGVGKSAL (SEQ ID NO:141), GAVGVGKSA (SEQ ID NO:155), LVVVGAVGVG (SEQ ID NO:156), LVVVGAVGV (SEQ ID NO:125), KLVVVGAVGV (SEQ ID NO:128), and KLVVVGAVG (SEQ ID NO:157); where the KRAS peptide has a length of 9 amino acids or 10 amino acids.

11. A T-cell modulatory polypeptide of claim 1, wherein:
A) the KRAS peptide is KLVVVGADGV (SEQ ID NO:127) and the MHC heavy chain polypeptide comprises an amino acid sequence having at least 95% amino acid sequence identity to an HLA-A*0201 polypeptide; or
B) the KRAS peptide is VVVGADGVGK (SEQ ID NO:111) or VVGAVGVGK (SEQ ID NO:110), and the MHC heavy chain polypeptide comprises an amino acid sequence having at least 95% amino acid sequence identity to an HLA-A11*01 polypeptide.

12. A TMP of claim 1, comprising in order from N-terminus to C-terminus:
i) the KRAS peptide;
ii) optionally a peptide linker;
iii) the β2M polypeptide;
iv) optionally a peptide linker;
v) the MHC class I heavy chain polypeptide;
vi) optionally a peptide linker;
vii) an Ig Fc polypeptide;
viii) a rigid peptide linker or a short flexible peptide linker; and
ix) the one or more immunomodulatory polypeptides, optionally wherein when two or more immunomodulatory polypeptides are present, a peptide linker is interposed between each of the two or more immunomodulatory polypeptide; or
i) the KRAS peptide;
ii) a flexible peptide linker;
iii) the β2M polypeptide;
iv) a flexible peptide linker;
v) the MHC class I heavy chain polypeptide;
vi) a flexible peptide linker;
vii) the Ig Fc polypeptide;
viii) a rigid peptide linker or a short flexible peptide linker;
ix) a first copy of the at least one immunomodulatory polypeptide;
x) a peptide linker; and
xi) a second copy of the at least one immunomodulatory polypeptide, and
optionally wherein the peptide linker between the KRAS peptide and the β2M polypeptide comprises a cysteine; or
i) the KRAS peptide;
ii) a flexible peptide linker;
iii) the β2M polypeptide;
iv) a flexible peptide linker;
v) the MHC class I heavy chain polypeptide;
vi) a rigid peptide linker or a short flexible peptide linker;
vii) a first copy of the at least one immunomodulatory polypeptide;
viii) a rigid peptide linker or a short flexible peptide linker;
ix) a second copy of the at least one immunomodulatory polypeptide,
x) a peptide linker; and
xi) the Ig Fc polypeptide; and
optionally wherein the peptide linker between the KRAS peptide and the β2M polypeptide comprises a cysteine; or
i) the KRAS peptide;
ii) optionally a peptide linker;
iii) the β2M polypeptide;
iv) optionally a peptide linker;
v) the MHC class I heavy chain polypeptide;
vi) optionally a peptide linker;
vii) the one or more immunomodulatory polypeptides;
viii) the rigid peptide linker or a short flexible peptide linker; and
ix) an Ig Fc polypeptide, and
optionally wherein the peptide linker between the KRAS peptide and the β2M polypeptide comprises a cysteine, and optionally wherein when two or more immunomodulatory polypeptides are present, a peptide linker is interposed between each of the two or more immunomodulatory polypeptide.

13. A TMP of claim 1, wherein each of the optional one or more additional peptide linkers, if present, is independently selected from the group consisting of:
i) CGGGS(GGGGS)n, (SEQ ID NO:220) where n is an integer from 1-10, optionally wherein n is 2, 3, or 4;
ii) GCGGS(GGGGS)n (SEQ ID NO:217), where n is an integer from 1-10, optionally wherein n is 2, 3, or 4;
iii) (GGGGS)n(GCGGS), where n is an integer from 1-10, optionally wherein n is 2, 3, or 4;
iv) (GGGGS)n where n is an integer from 1-10, optionally wherein n is 2, 3, 4, or 7; and
v) AAAGG.

14. A TMP of claim 1, wherein the TMP comprises at least one rigid peptide linker, and wherein each rigid peptide linker is independently selected from the group consisting of:
i) (AP)n, where n is an integer from 1-10, optionally where n is 2-6;
ii) (EP)n, where n is an integer from 1-10, optionally where n is 2-6;
iii) (KP)n, where n is an integer from 1-10, optionally where n is 2-6; and
iv) a peptide comprising EAAAK, optionally where n is 1 or 2.

15. A TMP of claim 1, wherein:
a) the β2M polypeptide is joined to the KRAS peptide by a first linker that comprises the sequence CGGGS(GGGGS)n (SEQ ID NO:220) or GCGGS(GGGGS)n (SEQ ID NO:217), where n is an integer from 1-10, optionally wherein n is 2 or 3;
b) the MHC heavy chain polypeptide comprises a Cys at residue 84 and a Cys at residue 236;
c) the β2M polypeptide comprises a Cys at residue 12;
d) the Cys at amino acid residue 12 of the β2M polypeptide is disulfide bonded to a Cys at amino acid residue 236 of the MHC heavy chain polypeptide;
e) a disulfide bond links the Cys in the linker between the KRAS peptide and the β2M polypeptide with a Cys substituted for Tyr84 of the MHC heavy chain polypeptide;
f) the β2M polypeptide is connected to the MHC heavy chain polypeptide by a (GGGGS)n linker, where n is 1, 2, 3, 4, 5, 6, 7, 8, 9, or 10, optionally wherein n is 3 or 7;
g) the MHC heavy chain polypeptide comprises an amino acid sequence having at least 95% amino acid sequence identity to an HLA-A polypeptide selected from the group consisting of an HLA-A*0201 polypeptide, an HLA-A*1101 polypeptide, an HLA-A*3303 polypeptide, an HLA-A*2401 polypeptide, an HLA-E polypeptide, and an HLA-G polypeptide;
h) the TMP comprises two immunomodulatory polypeptides that are the same, are in tandem, and comprise a variant of IL-2 that comprises i) an H16A substitution and an F42A substitution; or ii) an H16T substitution and an F42A substitution; and
i) the TMP comprises at least one rigid peptide linker, and wherein the at least one rigid peptide linker is selected from the group consisting of: i) (AP)n, where n is an integer from 1-10; ii) (EP)n, where n is an integer from 1-10, optionally wherein n is 2-6; iii) (KP)n, where n is an integer from 1-10; and iv) a peptide comprising EAAAK.

16. A TMP of claim 15, wherein:
a) the β2M polypeptide is joined to the KRAS peptide by a first linker that comprises the sequence GCGGS(GGGGS)n (SEQ ID NO:217), where n is 2;
f) the β2M polypeptide is connected to the MHC heavy chain polypeptide by a (GGGGS)n linker, where n is 3 or 7;
g) the MHC heavy chain polypeptide comprises an amino acid sequence having at least 95% amino acid sequence identity to an HLA-A*1101 polypeptide;
h) the TMP comprises two immunomodulatory polypeptides that are the same, are in tandem, and comprise a variant of IL-2 that comprises an H16A substitution and an F42A substitution, and wherein the two immunomodulatory polypeptides are connected by a peptide linker; and
i) the at least one rigid peptide linker comprises the amino acid sequence (AP)n, where n is an integer from 1-10, optionally wherein n is 2-6.

17. A TMP of claim 1, wherein the TMP comprises the amino acid sequence set forth in any one of SEQ ID NOs:69-80 and 82-90.

18. A TMP of claim 1, wherein the TMP comprises at least one short flexible peptide linker, and wherein each short flexible peptide linker is independently selected from the group consisting of flexible peptide linkers comprising a number of amino acids selected from the group consisting of 2-4 aas, 2-5 aas, 3-6 aas, 4-8 aas, 5-10 aas and 10-14 aas.

19. A homodimer comprising a first and second TMP of claim 1, wherein the first and second TMPs are the same and are covalently bound by one or more disulfide bonds between the Ig Fc polypeptides of the first and second TMPs.

20. A heterodimer comprising a first and second TMP of claim 1, wherein the first and second TMPs are not the same and are bound by one or covalent bonds.

21. A nucleic acid comprising a nucleotide sequence encoding a TMP according to claim 1.

22. A recombinant expression vector comprising the nucleic acid of claim 21.

23. A pharmaceutical composition comprising a TMP according to claim 1.

24. A method of selectively modulating the activity of T cell specific for a KRAS peptide epitope, the method comprising contacting the T cell with a T-cell modulatory polypeptide according to claim 1, wherein said contacting selectively modulates the activity of the epitope-specific T cell.

25. A method of treating a KRAS-associated cancer in a patient having the cancer, the method comprising administering to the patient an effective amount of a pharmaceutical composition comprising a TMP according to claim 1.

26. A method of claim 25, further comprising co-administering one or more immune checkpoint inhibitors to the patient, optionally wherein the immune checkpoint inhibitor is an antibody specific for PD-L1, PD-1, TIGIT, or CTLA4.
